(19) United States
(12) Patent Application Publication
(10) Pub. No.: US 2004/0092714 A1
(43) Pub. Date: May 13, 2004

(54) Osteoclast-associated receptor

(76) Inventors: Yongwon Choi, Byrn Mawr, PA (US); Nacksung Kim, Secane, PA (US)

Correspondence Address: DARBY & DARBY P.C., P. O. BOX 5257, NEW YORK, NY, 10150-5257, US

(21) Appl. No.: 10/363,300

(22) PCT Filed: Sep. 4, 2001
(86) PCT No.: PCT/US01/27502

(30) Foreign Application Priority Data
Sep. 5, 2000 (US) 60230152
Jul. 24, 2001 (US) 60307557

(51) Int. Cl. C07K 1/00

(52) U.S. Cl. 530/350

(57) ABSTRACT

This invention relates to methods and compositions that modulate the activity of cells, such as osteoclast cells, involved in the growth, development, repair, degradation and homeostasis of bone tissue. The compositions may therefore by used to modulate such processes and to treat bone growth related disorders (for example, osteoporosis and osteopetrosis). In particular, the invention provides a novel polypeptide, referred to as the Osteoclast Associated Receptor or OSCAR, that is specifically expressed by oteoclast cells and modulates osteoclast cell activity. OSCAR nucleic acids (including vectors), fusion polypeptides and OSCAR specific antibodies are also provided, as well as diagnostic and screening assays using such nucleic acids, polypeptide and antibodies.

### FIELD OF THE INVENTION

The present invention relates to a novel gene, referred to herein as the “Osteoclast Associated Receptor” gene or “OSCAR”, and its gene product. The OSCAR gene is specifically expressed by osteoclast cells. Accordingly, the invention also relates to methods of identifying and isolating osteoclast cells by identifying cells that specifically express the OSCAR gene or gene product.

The OSCAR gene and gene product are also involved in regulating or modulating the maturation of osteoclast cells. Accordingly, the invention further relates to methods and compositions for modulating or suppressing the maturation and/or activity of osteoclast cells. Such methods are useful, e.g., for treating osteoclast-related diseases such as osteoporosis and osteopetrosis. Accordingly, the invention also relates to methods and compositions for treating such diseases.

The invention also relates to screening methods for identifying compounds that bind to and/or modulate activity of an OSCAR gene or gene product and which can therefore be used to modulate the maturation and/or activity of osteoclast cells. Compounds that be identified by such screening methods, and therefore are also in the field of the present invention, include OSCAR ligands and transmembrane signal adapters.

### BACKGROUND OF THE INVENTION

The development and homeostasis of bone is controlled largely by two different cells types: osteoblasts and osteoclasts. The bone matrix is secreted by osteoblasts, cells that lie on the surface of the existing bone matrix and deposit fresh layers of bone onto it. Mature osteoclasts are multinucleated cells of monocyte/macrophage origin that reabsorb calcified bone matrix. Ordinarily, the activities of these two cell types are tightly coordinated to maintain the structure and integrity of bone in an organism. However, the mechanisms that regulate the activities of these two cell types remain poorly understood and are largely unknown.

A number of diseases and disorders are associated with abnormal bone growth or abnormal increases or decreases in bone mass. For example, osteopetrosis is a thickening of the bone matrix and has been associated with defects in osteoclast maturation which make them unable to absorb bone (see, for example, Kong et al. Nature, 1999, 397:315-323; Soriano et al., Cell 1991, 64:693-702; Iotsova et al., Nat. Med. 1997,3:1285-1289). By contrast, osteoporosis is a disease characterized by an increase in osteoclast activity, resulting in bones that are extremely porous, easily fractured, and slow to heal. Numerous other diseases and disorders that involve or are associated with abnormal bone growth and resorption are also known, including Paget's disease, osteogenesis imperfecta, fibrous dysplasia, hypophosphatasia, primary hyperparathyroidism, arthritis and periodontal disease to name a few. Additionally, osteolysis can be induced by many malignant tumors resident in or distant from bone, e.g., skeletal metastases in cancers of the breast, lung, prostate, thyroid, and kidney, humoral hypercalcemia during malignancy, and multiple myelomas.

Such diseases and disorders represent a major public health concern in the United States and in other countries. For example, it has been estimated that 10 million Americans, 80% of whom are women, are already afflicted with osteoporosis, while another 10 million individuals have low bone mass and are therefore at an increased risk for the disease.

There exists, therefore, a need for methods and compositions that can be used to identify cells such as osteoblast and/or osteoclast (for example in cell or tissue samples), and regulate or modulate the activities of such cells. There also exists a need for methods and compositions to treat diseases and disorders associated with abnormal bone growth and resorption, including the diseases discussed above, for example by modulating the activities of osteoblast and osteoclast cells. These and other needs in the art are addressed by the present invention.

### SUMMARY OF THE INVENTION

The present invention overcomes the above-discussed and other problems in the art by providing compositions and methods that are involved in processes associated with the growth, development, repair, resorption degradation or homeostasis of bone tissue and are therefore useful for the modulation of such processes. For example, the methods of the invention may be useful for the treatment of disorders that involve abnormal growth, development, repair, resorption, degradation, resorption or homeostasis of bone tissue (i.e., “bone growth related disorders”). Examples of such disorders include, but are not limited to, osteoporosis and osteopetrosis. Other non-limiting examples of such disorders include Paget's disease, osteogenesis imperfecta, fibrous dysplasia, hypophosphatasia, primary hyperparathyroidism, arthritis, periodontal disease and osteolysis (e.g., from malignant tumors).

In particular, the present invention provides novel polypeptides, referred to herein as OSCAR polypeptides, which are expressed by osteoclast cells. The OSCAR polypeptides of the invention also modulate the growth and maturation of osteoclast, as well as activities, such as the resorption of bone tissue, that are associated with osteoclast cells.

In certain preferred embodiments, the invention provides OSCAR polypeptides that are murine (i.e., mouse) polypeptides and are expressed by murine osteoclast cells. For example, in one embodiment, the invention provides OSCAR polypeptides that comprise the amino acid sequence set forth in FIG. 2B (SEQ ID NO:3). In another embodiment, the invention provides OSCAR polypeptides comprising the amino acid sequence set forth in FIG. 1C (SEQ ID NO:3). In yet another preferred embodiment, the invention provides OSCAR polypeptides comprising the amino acid sequences set forth in FIGS. 26B and 27B (SEQ ID NOS: 29 and 31, respectively). In other preferred embodiments, the invention provides OSCAR polypeptides that are human polypeptides. For example, in preferred embodiments the OSCAR polypeptides of the invention are polypeptides encoded by the genomic sequence set forth in FIGS. 7A-D (SEQ ID NO:12). In certain particularly preferred embodiments, an OSCAR polypeptide of the invention may comprise the amino acid sequence set forth in FIG. 3B (SEQ ID NO:7), in FIG. 4B (SEQ ID NO:9), FIG. 5B (SEQ ID NO:11), FIG. 24B (SEQ ID NO: 25) or in FIG. 25B (SEQ ID NO: 27). In still other embodiments, the invention provides polypeptides, including fusion polypeptides, that comprise an amino acid sequence corresponding to one or more domains of a full length OSCAR polypeptide, such as a signal peptide sequence, an Ig-like domain sequence, a transmembrane domain sequence, a cytoplasmic tail domain sequence or any combination thereof for a full length OSCAR polypeptide (e.g., from any of the polypeptides set forth in FIGS. 1C, 3B, 4B, 5B, 24B, 25B, 26B and 27B and in SEQ ID NOS:3, 7, 9, 11, 25, 27, 29 and 31respectively). In still other embodiments, the invention provides variants of an OSCAR polypeptide. In particular, the invention provides polypeptides which are encoded by a nucleic acid that hybridizes, under defined hybridization conditions, to the complement of an OSCAR polypeptide, e.g., as provided in FIGS. 1C, 2B, 3B, 4B, 5B, 24B, 25B, 26B and 27B and in SEQ ID NOS:3, 7, 9, 11, 25, 27, 29 and 31respectively)

The invention additionally provides nucleic acids that encode OSCAR polypeptides of the invention, including, for example, nucleic acids comprising the nucleotide sequence provided in FIGS. 1A-B, 2A, 3A, 4A, 5A, 24A, 25A, 26A and 27A (SEQ ID NOS:1-2, 4, 6, 8, 10, 26, 28, 30 and 32, respectively), as well as the genomic OSCAR nucleic acid sequences set forth in FIGS. 7A-D (SEQ ID NO:12). The invention further provides vectors and host cells that comprise these nucleic acids, and antibodies that specifically bind to those OSCAR polypeptides and OSCAR nucleic acids. The invention also relates to fragments of such OSCAR polypeptides, nucleic acids and antibodies.

In addition, the present invention also relates to and provides screening assays for detecting and identifying OSCAR nucleic acids and OSCAR polypeptides of the invention, including screening assays for detecting the presence or expression of OSCAR nucleic acids and OSCAR polypeptides in cells, on the surface of cells (e.g., OSCAR expressed on cell surfaces) in cell cultures (e g., in the cell culture media), in cell culture extracts or in cell lysates. These methods include methods for detecting and identifying variant OSCAR polypeptides and nucleic acids: for example OSCAR polypeptides which comprise one or more amino acid substitutions, deletions or insertions; or nucleic acids that encode an OSCAR polypeptide having one or more amino acid substitutions, insertions or deletions. Other variant OSCAR polypeptides and nucleic acids that may be identified by these methods include homologous OSCAR polypeptides and nucleic acids (e.g., from other species of organism, and preferably from other mammalian organisms such as from humans). Such variant OSCAR polypeptides and nucleic acids, as well as antibodies that specifically bind thereto and fragments thereof, are therefore provided by and considered part of the present invention.

The present invention further provides methods (e.g., screening assays) for identifying compounds that specifically bind to an OSCAR nucleic acid of the invention or to an OSCAR polypeptide of the invention. Compounds that may be identified by such screening assays include small molecules (e.g., molecules less than about 2 kD, and more preferably less than about 1 kD in molecular weight) and macromolecules, including proteins, peptides and polypeptides. Compounds that may be identified by such screening methods further include intracellular compounds, such as natural ligands, that specifically bind to an OSCAR gene or to an OSCAR gene product (e.g., to an OSCAR nucleic acid or to an OSCAR polypeptide). In addition, other screening assays are provided for identifying compounds (including small molecules, macromolecules, proteins, peptides and polypeptides) that interfere with the binding interaction between an OSCAR polypeptide and a specific binding partner (e.g., an OSCAR-specific ligand), or between an OSCAR nucleic acid and a specific binding partner. Such screening methods are therefore considered part of the present invention. In addition, compounds which are identified by these assays, including binding compounds (e.g., OSCAR-specific ligands) and compounds that interfere with OSCAR-specific binding interactions are also part of the present invention.

In another aspect, the present invention provides methods for modulating osteoclast cell activities. Such methods generally comprise contacting an osteoclast cell with a compound that modulates activity of an OSCAR gene (for example, expression of an OSCAR gene) or of an OSCAR gene product. The compounds used in these methods include OSCAR antagonists, which inhibit OSCAR signaling and therefore inhibit osteoclast cell activation (for example, maturation), as well as OSCAR agonists (including OSCAR specific ligands), which promote OSCAR signaling and/or maturation of osteoclast cell and osteoclast cell activity. These methods may comprise contacting an osteoclast cell with a compound (for example, an antisense, ribozyme, triple-helix forming nucleic acid, or other small compound) so that expression of an OSCAR gene or an OSCAR gene product by the cell is enhanced or inhibited. Such methods may include methods for increasing osteoclast cell activity, for example, by contacting an osteoclast cell with a compound that binds to and/or increases the activity of an OSCAR gene product. In one preferred embodiment of this method, an osteoclast cell is contacted with an OSCAR-specific ligand.

The methods of the invention further include decreasing activity of an osteoclast cell. These methods may comprise contacting an osteoclast cell with a compound that inhibits or decreases the activity of an OSCAR gene product. In certain preferred embodiments, the compound may be one that inhibits or interferes with the binding of an OSCAR-specific ligand to an OSCAR gene product. For example, in one preferred embodiment the compound comprises an antibody that specifically binds to either an OSCAR gene product or to an OSCAR-specific ligand so that binding between the OSCAR-specific ligand and the OSCAR gene product is inhibited. In another preferred embodiment, the compound comprises one or more soluble OSCAR polypeptide amino acid sequences, most preferably including amino acid sequence that comprises a ligand-binding domain of an OSCAR polypeptide (e.g., the extracellular and/or signal sequence domain). In a particularly preferred embodiment, the compound administered comprises a soluble fusion polypeptide having these amino acid sequences in conjunction with an immunoglobulin Fc region or other small molecules.

### BRIEF DESCRIPTION OF THE DRAWINGS

FIGS. 1A-1C shows the cDNA sequences of the 1.8 kb (FIG. 1A; SEQ ID NO:1) and 1.1 kb (FIG. 1B; SEQ ID NO:2) splice variants of the murine OSCAR gene. The start (ATG) and stop (TGA) codons of each sequence are indicated in bold-faced type. The OSCAR polypeptide sequence encoded by both cDNA transcripts is set forth in FIG. 1C (SEQ ID NO:3).

FIGS. 2A-2B shows the cDNA sequence (FIG. 2A; SEQ ID NO:4) of the murine OSCAR fragment contained in the clone OCL178. The amino acid sequence of the OSCAR polypeptide fragment encoded by this clone, which corresponds to the sequence of amino acid residues 161-265 of SEQ ID NO:3, is set forth in FIG. 2B (SEQ ID NO:5).

FIGS. 3A-3B show the cDNA sequence (FIG. 3A; SEQ ID NO:6) and predicted amino acid sequence (FIG. 3B; SEQ ID NO:7) of the C18 isoform of a human OSCAR gene and its gene product.

FIGS. 4A-4B show the cDNA sequence (FIG. 4A; SEQ ID NO:8) and predicted amino acid sequence (FIG. 4B; SEQ ID NO:9) of the C16 isoform of a human OSCAR gene and its gene product.

FIGS. 5A-5B show the cDNA sequence (FIG. 5A; SEQ ID NO:10) and predicted amino acid sequence (FIG. 5B; SEQ ID NO:11) of the C10 isoform of a human OSCAR gene and its gene product.

FIG. 6 shows an amino acid sequence alignment of the murine OSCAR polypeptide sequence set forth in FIG. 1C (SEQ ID NO:3) and the C18 isoform of a human OSCAR polypeptide set forth in FIG. 3B (SEQ ID NO:7). The murine and human OSCAR polypeptide sequences are denoted mOSCAR (top line) and hOSCAR (bottom line), respectively.

FIGS. 7A-7D set forth the sequence of nucleotide residues 117001-124920 (SEQ ID NO:12) from the human chromosome 19 clone CTD-3093 (GenBank Accession No. AC012314.5; GI:7711547) from which a human OSCAR gene was isolated using the BLASTN algorithm. Exon sequences of the human OSCAR gene are indicated in upper-case characters. The translation (i e., protein coding) regions of the human OSCAR gene are underlined.

FIGS. 8A-8B show Southern Blot analysis of plasmid DNA from 250 randomly selected clones in a substraction (murine OC minus MØ) cDNA library using total cDNA probes from bone marrow derived macrophages (FIG. 8A) and bone-marrow derived osteoclast cells (FIG. 8A).

FIG. 9 shows results of Northern Blot assays in which labeled cDNA from the murine OSCAR fragment OCL178 (top), the osteoclast specific gene TRAP (middle) and the osteoclast specific gene Cathepsin K (bottom), respectively, was hybridized to mRNA derived from bone-marrow derived macrophages (BMM), bone-marrow derived osteoclast cells (BMOC) and bone-marrow derived dendritic cells (BMDC).

FIG. 10 shows results of Northern Blot assays in which labeled cDNA from the murine OSCAR fragment OCL178 (top), and the osteoclast specific genes TRAP (middle) and Cathepsin K (bottom) was hybridized to mRNA derived from a variety of different tissues, including muscle, kidney, brain, heart, liver, lung, intestine, thymus, spleen, lymph node, and osteoclast (OCL).

FIGS. 11A-11C show Northern Blot assays in which labeled cDNA from the murine OSCAR fragment OCL178 hybridized to mRNA derived from bone-marrow derived macrophages (BMM) and osteoclast cells (OCL), compared to mRNA from RAW264.7 cells (RAW) that differentiate into osteoclast-like cells by in vitro treatment with TRANCE. FIG. 11A compares the Norther Blot with mRNA from macrophage and osteoclast cells with RAW264.7 cell mRNA extracted 0, 3 and 24 hours post TRANCE administration. FIG. 11B shows Northern Blots from RAW264.7 cell mRNA 1, 2, 3 and 4 days post TRANCE administration. FIG. 11C compares Northern Blots of mRNA extracted from skull and long bone tissue with mRNA from bone-marrow derived osteoclast cells (BMOC).

FIGS. 12A-12B shows Southern Blot analysis of EcoRI and BglII digested mouse (FIG. 12A) and human (FIG. 12B) genomic DNA using a labeled murine OSCAR nucleotide probe.

FIGS. 13A-13B show results from FACS analysis of primary osteoblast cells stained with an isotype control human IgG1 (FIG. 13A) or with a soluble OSCAR-Ig fusion polypeptide (FIG. 13B), followed by PE-conjugated anti-human IgG1 antibody.

FIG. 14 shows a chart indicating the numbers of TRAP(+) multinucleated osteoclast cells observed when bone marrow cells were co-cultured with osteoblast cells and treated with the indicated amount of vitamin D₃, either in the presence of a soluble OSCAR-Ig fusion polypeptide (▪), or in the presence of human IgG1 (□).

FIGS. 15A-15C graphically present data from kinetics experiments where the number of TRAP(+) multi-nuclear osteoclast cells were counted in co-cultures of osteoclast precursors with osteoblast cells (FIGS. 15A-C) after being incubated for 6, 7, 8 and 9 days in the presence of vitamin D₃ (□), vitamin D₃ and a soluble OSCAR-Ig fusion polypeptide (⋄), or with vitamin D₃ and a control IgG1 polypeptide (∘). In FIGS. 15A-B total bone marrow cells were used for osteoclast precursors while in FIG. 15C, M-CSF-dependent bone marrow floater cells were used for co-culture experiments. FIG. 15B is a bar graph indicating the number of TRAP (+) multi-nucleated osteoclasts observed in the co-culture experiments after 7 days incubation.

FIGS. 16A-16J are photomicrographs from a dentine resorption assay using co-cultures of murine bone marrow cells and osteoblast cells (see, Tamura et al., J. Bone Miner. Res. 1993, 8:953-960). FIGS. 16A-16E are photomicrographs of the TRAP(+) osteoclasts on dentine slices. FIGS. 16F-16J are photomicrographs of the corresponding dentine slices after cells were removed. Dark stains in these micrographs indicate regions where dentine has been resorbed. In more detail, FIGS. 16A and 16F show TRAP(+) cells and dentine slices, respectively, that were incubated in growth medium alone. FIGS. 16B and 16G are photomicrographs of TRAP(+) cells (FIG. 16B) and dentine slices (FIG. 16G) that were incubated with vitamin D₃. Photomicrographs of TRAP(+) osteoclast cells and dentine slices that were incubated with vitamin D₃ and a soluble murine OSCAR-Ig fusion polypeptide are shown in FIGS. 16C and 16H, respectively. FIGS. 16D and 16I are photomicrographs of TRAP(+) osteoclast cells (FIG. 16D) and dentine slices (FIG. 16I) that were incubated with vitamin D₃ and a TR-Fc fusion polypeptide. FIGS. 16E and 16J are photomicrographs of TRAP(+) cells and dentine slices, respectively, that were incubated with vitamin D₃ and a control IgG1 fusion polypeptide.

FIG. 17 is a bar graph presenting the quantitative results from the dentine resorption data shown in FIGS. 16A-16J. Resorption pits are counted for dentine slices on which co-cultures of murine osteoclast precursors and osteoblast cells were incubated in growth medium alone (“medium”), with vitamin D₃ (“Vit.D3”), vitamin D₃ and OSCAR-Ig (Vit.D3+OSCAR-IgG”), or with vitamin D₃ and a control IgG1 polypeptide (Vit.D3+IgG).

FIGS. 18A and 18B present data from experiments with human monocyte cell cultures that were incubated: (a) M-CSF alone (“M”); (b) M-CSF and TRANCE (“MT”); (c) M-CSF, TRANCE and a soluble human OSCAR-Ig fusion polypeptide (“MT+hOSCAR-IgG”); M-CSF, TRANCE and a soluble murine OSCAR-Ig fusion polypeptide (“MT+mOSCAR-IgG”); (c) M-CSF, TRANCE and a control IgG1 polypeptide (“MT+IgG”); and (d) M-CSF, TRANCE and a TR-Fc fusion polypeptide (“MT+TR-Fc”). The numbers of TRAP(+) multi-nuclear osteoclasts were counted in each culture after incubation for five (FIG. 18A) and ten days (FIG. 18B).

FIGS. 19A-19F show photomicrographs of human monocyte cells that were incubated for five days: in the presence of M-CSF (FIG. 19A); with M-CSF and TRANCE (FIG. 19B); with M-CSF, TRANCE and a soluble human OSCAR-Ig fusion polypeptide (FIG. 19C); in the present of M-CSF, TRANCE and a soluble murine OSCAR-Ig fusion polypeptide (FIG. 19D); with M-CSF, TRANCE and a TR-Fc fusion polypeptide (FIG. 19E); and with M-CSF, TRANCE and a human IgG1 polypeptide (FIG. 19F). Multi-nuclear TRAP(+) osteoclasts are indicated by the arrows in FIGS. 19B and 19F.

FIGS. 20A-20F show photomicrographs of human monocyte cell cultures that were incubated for ten days: in the presence of M-CSF (FIG. 20A; with M-CSF and TRANCE (FIG. 20B); with M-CSF, TRANCE and a soluble human OSCAR-Ig fusion polypeptide (FIG. 20C); in the present of M-CSF, TRANCE and a soluble murine OSCAR-Ig fusion polypeptide (FIG. 20D); with M-CSF, TRANCE and a TR-Fc fusion polypeptide (FIG. 20E); and with M-CSF, TRANCE and a human IgG1 polypeptide (FIG. 20F). Multi-nuclear TRAP(+) osteoclasts are indicated by the arrows in FIGS. 20B and 20F.

FIGS. 21A-21J are photomicrographs from a dentine resorption assay (Tamura et al., J. Bone. Miner. Res. 1993, 8:953-960) using human monocyte cells. FIGS. 21A-21E are photomicrographs of the TRAP(+) human osteoclasts cultured on dentine slices. FIGS. 21F-21J are photomicrographs of the corresponding dentine slices after cells were removed. Dark stains in these micrographs indicate regions where dentine has been resorbed. In more detail, FIGS. 21A and 21F show TRAP(+) human cells and dentine slices, respectively, that were incubated in the presence of M-CSF alone. FIGS. 21B and 21G are photomicrographs of TRAP(+) human cells (FIG. 21B) and the corresponding dentine slices (FIG. 21G) that were incubated with M-CSF and TRANCE. Photomicrographs of TRAP(+) human cells that were incubated in the presence of a soluble murine OSCAR-Ig fusion polypeptide are shown in FIGS. 21C and 21H, respectively. FIGS. 21D and 21I are photomicrographs of TRAP(+) human cells (FIG. 21D) and the corresponding dentine slice (FIG. 21I) that were incubated with a TR-Fc fusion polypeptide. FIGS. 21E and 21J are photomicrographs of TRAP(+) human cells (FIG. 21E) and the corresponding dentine slice (FIG. 21J) that were incubated with an IgG1 polypeptide.

FIGS. 22A-22F show photomicrographs from co-cultures of murine osteoblast and bone marrow cells that were incubated for six days: in growth medium alone (FIG. 22A); with vitamin D₃ (FIG. 22B); with vitamin D₃ and a human OSCAR-Ig fusion polypeptide (FIG. 22C); with vitamin D₃ and a murine OSCAR-Ig fusion polypeptide (FIG. 22D); with vitamin D₃ and a TR-Fc fusion polypeptide (FIG. 22E); and with vitamin D₃ and a human IgG1 polypeptide (FIG. 22F).

FIG. 23 graphically presents quantitative data from murine co-culture experiments shown in FIGS. 22A-22F. Specifically, number of mature TRAP(+) multi-nuclear osteoclasts are indicated for each co-culture described supra, for FIGS. 22A-22F.

FIGS. 24A-B show the cDNA sequence (FIG. 24A; SEQ ID NO:26) and predicted amino acid sequence (FIG. 24B; SEQ ID NO:25) of the S1 splice variant of a human OSCAR gene and its gene product. The start (ATG) and stop (TGA) codons of each sequence are indicated in bold-faced type.

FIGS. 25A-B show the cDNA sequence (FIG. 25A; SEQ ID NO:28) and predicted amino acid sequence (FIG. 25B; SEQ ID NO:27) of the S2 splice variant of a human OSCAR gene and its gene product. The start (ATG) and stop (TGA) codons of each sequence are indicated in bold-faced type.

FIG. 26A shows the cDNA sequences of the M3 splice variant of the murine OSCAR gene (FIG. 26A; SEQ ID NO:30). The start (ATG) and stop (TGA) codons of each sequence are indicated in bold-faced type. The OSCAR polypeptide sequence encoded by both cDNA transcripts is set forth in FIG. 26B (SEQ ID NO:29).

FIG. 27A shows the cDNA sequences of the M4 splice variant of the murine OSCAR gene (FIG. 27A; SEQ ID NO:32). The start (ATG) and stop (TGA) codons of each sequence are indicated in bold-faced type. The OSCAR polypeptide sequence encoded by both cDNA transcripts is set forth in FIG. 27B (SEQ ID NO:31).

### DETAILED DESCRIPTION OF THE INVENTION

The present invention relates to a novel gene, referred to herein as the “Osteoclast Associated Receptor” or OSCAR gene, and its gene products. The OSCAR gene and its gene product, which are described herein for the first time, are specifically expressed in osteoclast cells. Further, Applicants have also discovered the existence of an OSCAR specific ligand, referred to herein as an “OSCAR ligand” or “OSCAR-L”, that is produced by osteoblast cells. OSCAR specific ligands of the invention may also be expressed by other cells, including, for example, murine embryonic fibroblasts, murine NIH 3T3 fibroblasts, murine ST2 osteoblast-like cells, Mink lung epithelial cells, at UMR106 osteoblast-like cells, human HEK293 and HEK293T cells, hFOB1.19, and monkey COS-1 cells. The OSCAR ligand binds to the OSCAR gene product. In experiments which are described in the Examples presented below, contacting immature osteoclast cells with osteoblast cells that express an OSCAR ligand effectively stimulates osteoclast maturation, increasing the number of mature multinucleated osteoclast cells. However, the administration of soluble fusion proteins of the OSCAR gene product inhibits binding of the OSCAR ligand to OSCAR polypeptides expressed by these osteoclast cells, and thereby inhibits maturation of the osteoclast cells. Thus, the OSCAR gene and its gene product can be used to modulate (i.e., to increase or decrease) osteoclast activity and are therefore useful, e.g., in methods of treating diseases and disorders associated with abnormal bone growth, including osteoporosis and osteopetrosis.

An OSCAR polypeptide is, in general, a polypeptide that is encoded by a gene which hybridizes to the complement of an OSCAR nucleic acid sequence as described, infra. Typically, a full-length OSCAR polypeptide of the invention has an apparent molecular weight of about 35 kDa or, alternatively, about 40 kDa. An OSCAR polypeptide of the invention may also regulate the maturation of osteoclast cells as described in the Examples, infra.

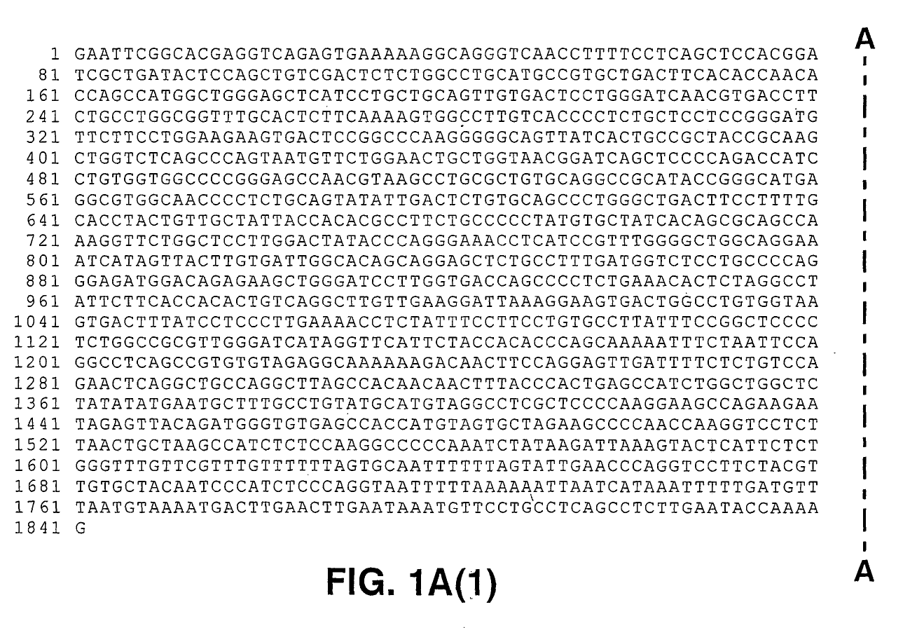

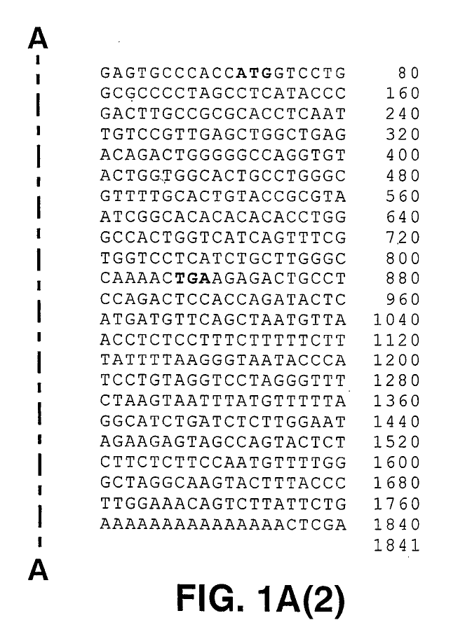

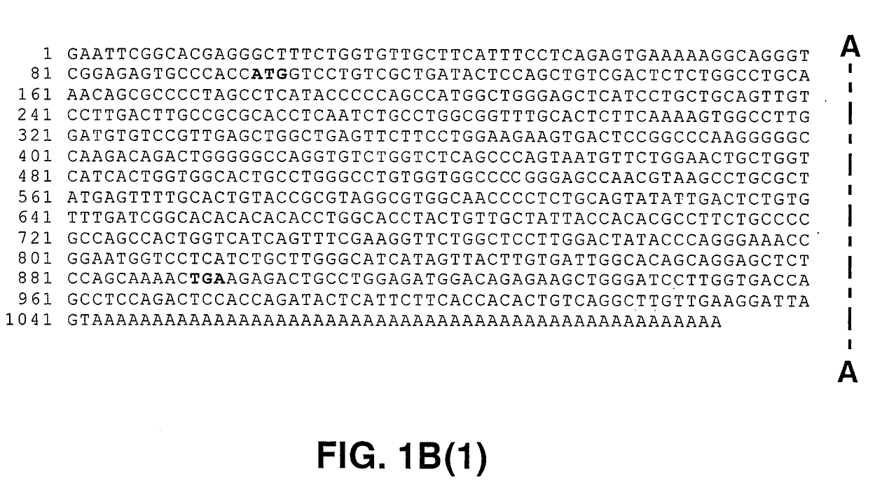

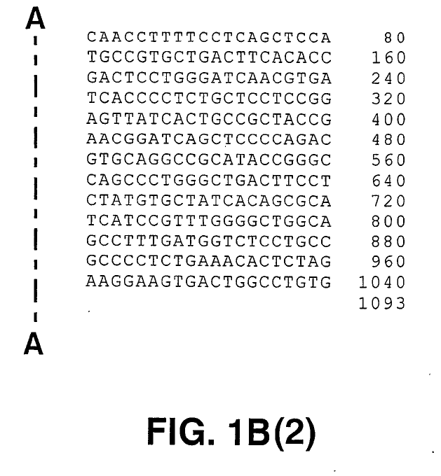

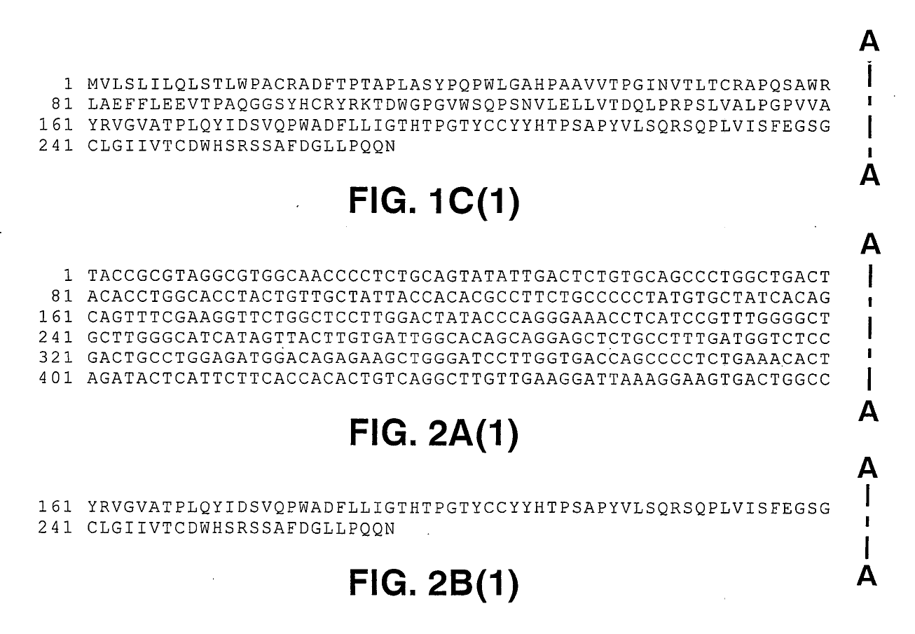

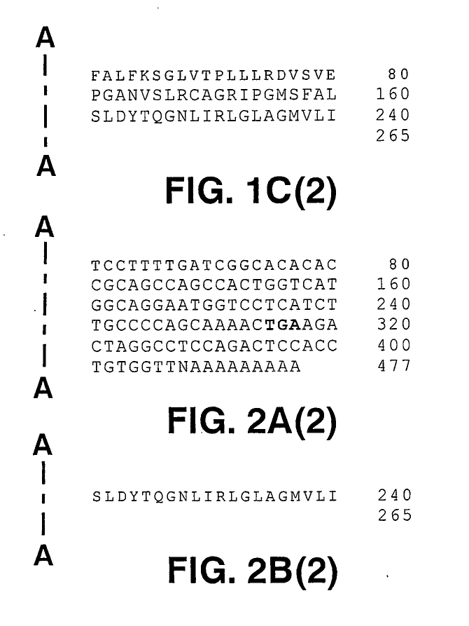

The OSCAR polypeptide is further characterized as an immunoglobulin superfamily member comprising two immunoglobulin domains and a transmembrane domain, as described in detail below. Thus, in preferred embodiments OSCAR polypeptides of the invention share amino acid sequence homology and/or amino acid sequence identity with other immunoglobulin proteins and polypeptides, such as murine PirA and bovine FcαR. For example, and not by way of limitation, a search of the NCBI protein database using the BLASP algorithm (standard parameters) to identify polypeptides that are similar to the particular OSCAR polypeptide set forth in FIG. 1C reveals that the polypeptide shares 26.4% sequence identity with murine PirA6 (GenBank Accession No. AAC53217.1) and 24.2% sequence identity with the polypeptide bovine FcαR (GenBank Accession No. P24071).

The OSCAR polypeptide can also be characterized by its expression pattern in cells. In particular, the OSCAR polypeptide is preferably expressed specifically by osteoclast cells and preferably is not expressed by any other cell type, with the exception of those host cells that have been transformed to express the OSCAR polypeptide. In particular, OSCAR polypeptides of the invention preferably are not expressed by other bone-marrow derived cells including macrophages and dendritic cells.

In one specific embodiment, an OSCAR polypeptide of the invention is derived from a murine (i.e., mouse) cell or has an amino acid sequence of a peptide derived from a murine cell. For example, a murine OSCAR polypeptide of the invention may comprise the amino acid sequence set forth in FIG. 1C (SEQ ID NO:3). This sequence comprises sequences corresponding to at least five distinct domains: a signal peptide sequence (comprising amino acid residues 1-16 of SEQ ID NO:3), two Ig-like domain sequences (comprising amino acid residues 17-122 and 123-228, respectively, of SEQ ID NO:3), a transmembrane domain sequence (comprising amino acid residues 229-247 of SEQ ID NO:3) and a cytoplasmic tail domain sequence (comprising amino acid residues 248-264 of SEQ ID NO:3).

In various aspects of this specific embodiment, a mature OSCAR polypeptide lacks a signal peptide sequence. Thus, in another specific embodiment, an OSCAR polypeptide of the invention comprises an amino acid sequence corresponding to amino acid residues 17-264 of the sequence set forth in FIG. 1C (SEQ ID NO:3). In still other embodiments, soluble OSCAR polypeptides of the invention lack a transmembrane domain and (in most embodiments) a cytoplasmic tail domain. Accordingly, in still other specific embodiments an OSCAR polypeptide of the invention comprises an amino acid sequence corresponding to amino acid residues 17-228 and, optionally, amino acid residues 248-264 of the sequence set forth in FIG. 1C (SEQ ID NO:3).

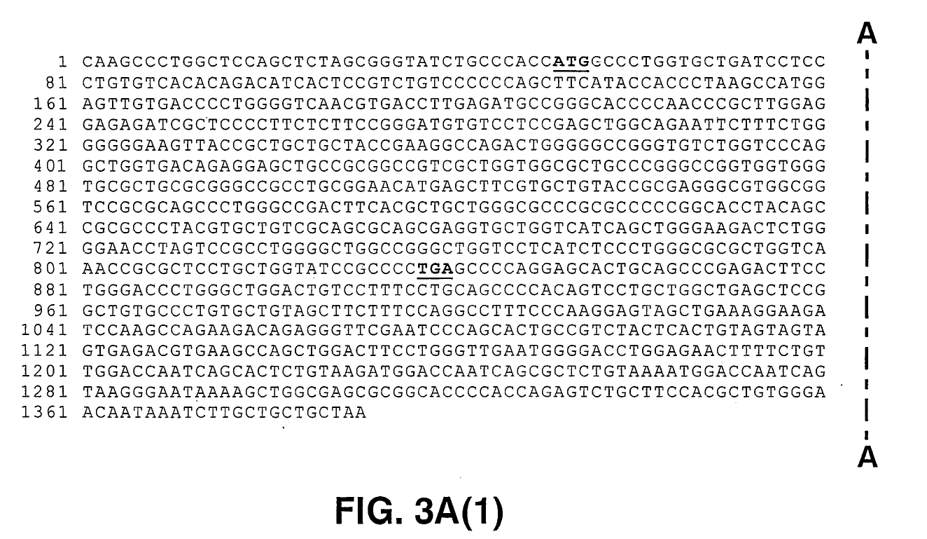

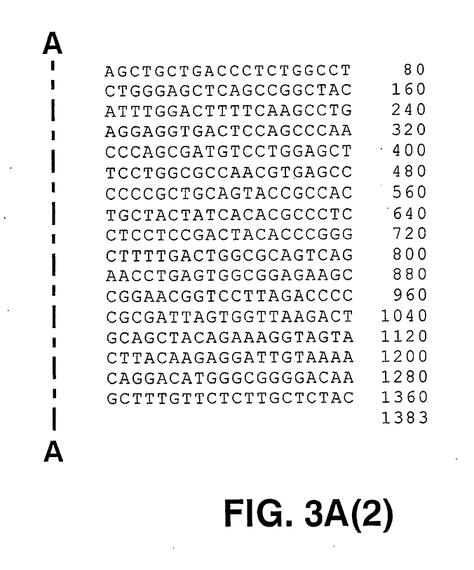

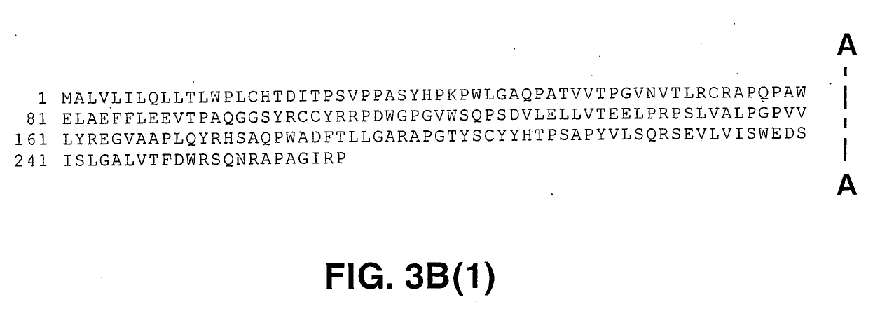

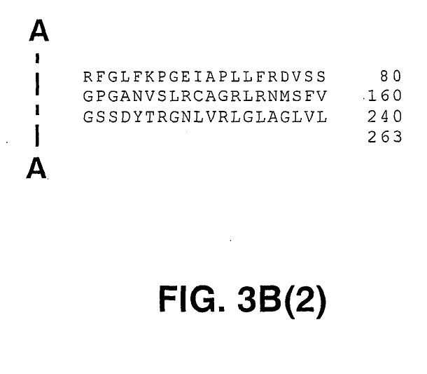

In other specific embodiments, an OSCAR polypeptide of the invention is derived from a human cell or substantially corresponds to a polypeptide derived from a human cell. For example, a human OSCAR polypeptide of the invention may comprise the amino acid sequence of the polypeptide referred to herein as “the C18 human OSCAR isoform” and having the amino acid sequence set forth in FIG. 3B (SEQ ID NO:7). Preferably, amino acid residue 97 of the C18 human OSCAR amino acid sequence is a serine (Ser or S), as indicated in FIG. 3B (SEQ ID NO:7). However, in another exemplary embodiment, amino acid residue 97 of that sequence can be an isoleucine (Ile or I). The C18 human OSCAR amino acid sequence also comprises amino acid sequences corresponding to at least four domains, which correspond to the four domains described above for the murine OSCAR polypeptide depicted in FIG. 1C (SEQ ID NO:3) In particular, the C18 human OSCAR isoform comprises a signal peptide sequence (comprising amino acid residues 1-18 of SEQ ID NO:7), two Ig-like domain sequences (comprising amino acid residues 19-123 and 124-229, respectively, of SEQ ID NO:7) a transmembrane domain sequence (comprising amino acid residues 230-248 of SEQ ID NO:7) and a cytoplasmic tail domain sequence (comprising amino acid residues 249-263 of SEQ ID NO:7).

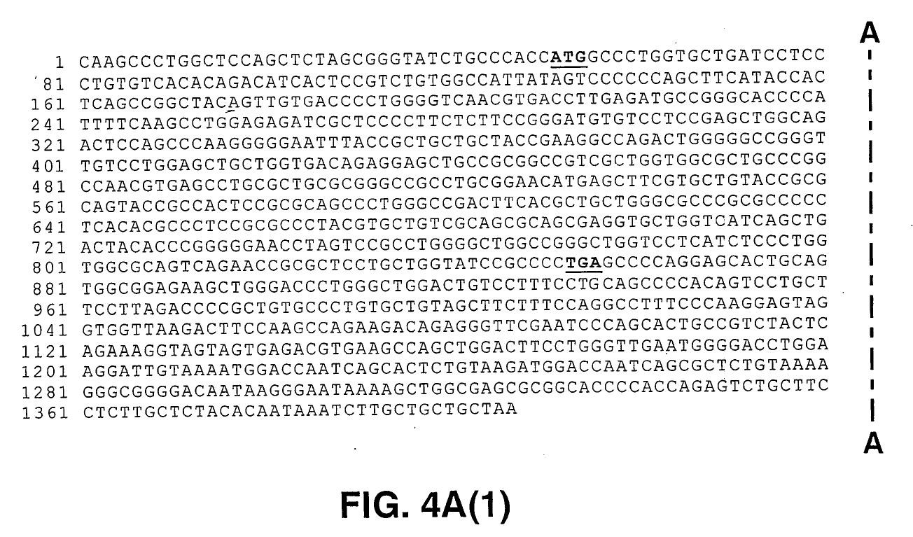

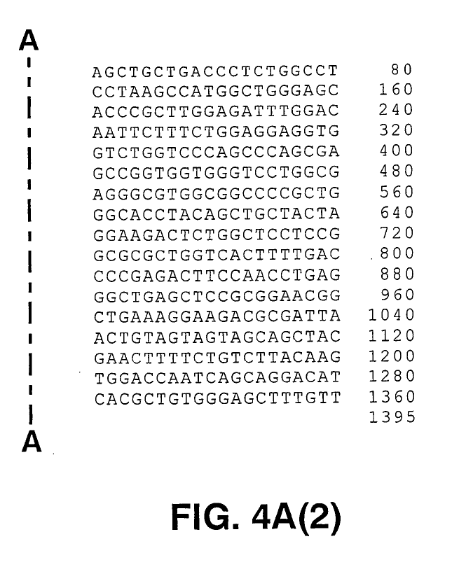

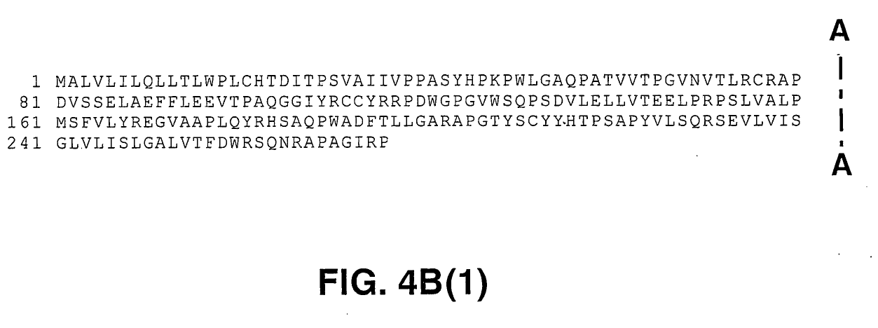

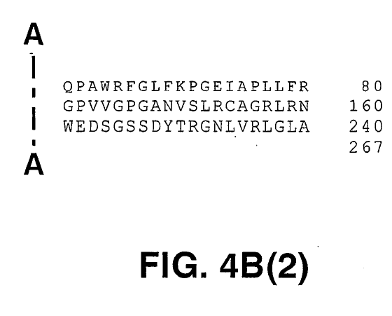

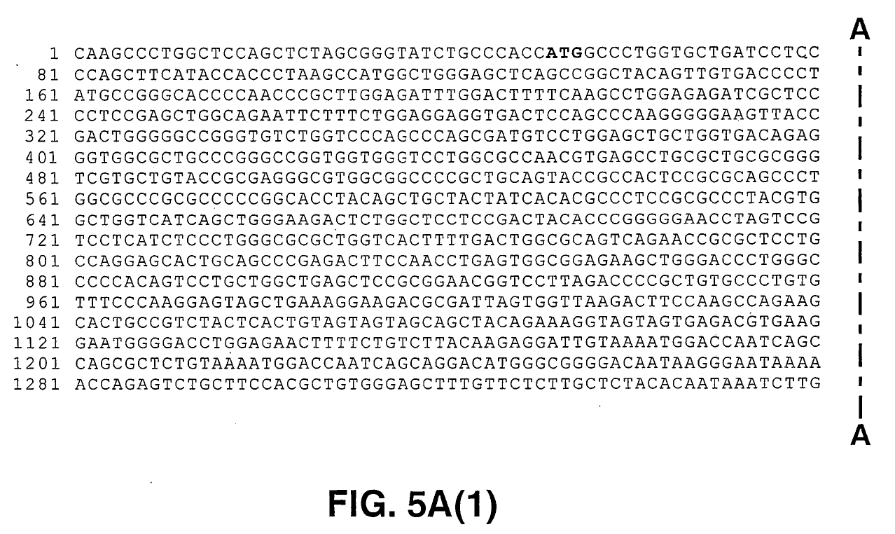

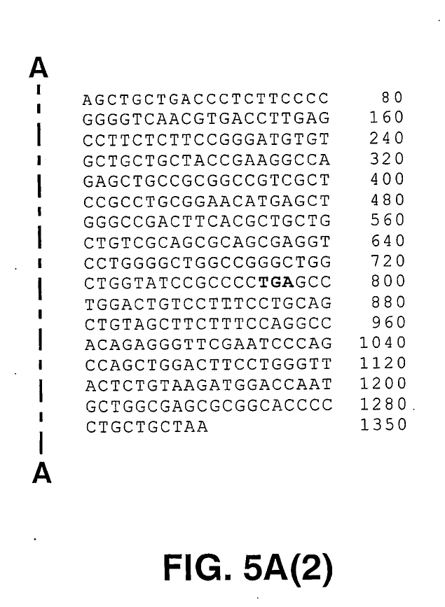

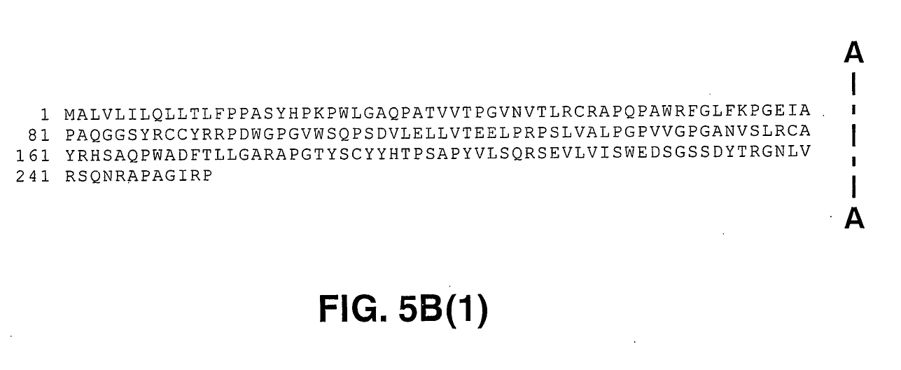

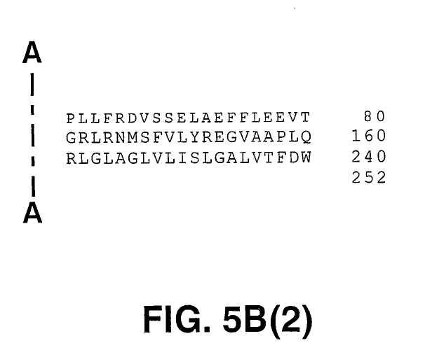

Alternatively, a human OSCAR polypeptide of the invention may comprise the amino acid sequence of the polypeptide referred to herein as “the C16 human OSCAR isoform” and having the amino acid sequence set forth in FIG. 4B (SEQ ID NO:9). In yet another specific embodiment, a human OSCAR polypeptide of the invention may comprise the amino acid sequence of the polypeptide referred to herein as “the C10 human OSCAR isoform” and having the amino acid sequence set forth in FIG. 5B (SEQ ID NO:11). Preferably, amino acid residue 86 of the C10 human OSCAR amino acid sequence is a serine (Ser or S), as indicated in FIG. 5B (SEQ ID NO:11). However, in another exemplary embodiment amino acid residue 86 of that sequence can be an isoleucine (I or Ile). Each of these human OSCAR polypeptides comprises amino acid sequences corresponding to at least four domains which correspond to the domains described supra for the murine OSCAR polypeptide depicted in FIG. 1C (SEQ ID NO:3) and for the C18 human OSCAR isoform depicted in FIG. 3B (SEQ ID NO:7).

In particular, the C16 human OSCAR isoform comprises a signal peptide sequence (comprising amino acid residues 1-18 of SEQ ID NO:9), two Ig-like domain sequences (comprising amino acid residues 19-127 and 128-233, respectively, of SEQ ID NO:9), a transmembrane domain sequence (comprising amino acid residues 234-252 of SEQ ID NO:9) and a cytoplasmic tail domain sequence (comprising amino acid residues 253-267 of SEQ ID NO:9).

The C10 human OSCAR isoform also comprises a signal peptide sequence (comprising amino acid residues 1-13 of SEQ ID NO:11), two Ig-like domain sequences (comprising amino acid residues 14-112 and 113-218, respectively, of SEQ ID NO:11), a transmembrane domain sequence (comprising amino acid residues 219-237 of SEQ ID NO:11) and a cytoplasmic tail domain sequence (comprising amino acid residues 238-252 of SEQ ID NO:11).

As described supra for the murine OSCAR polypeptides of the invention, a mature human OSCAR polypeptide may, in various aspects of these embodiments, lack a signal peptide sequence. Thus, in other specific embodiments, a human OSCAR polypeptide of the invention may comprise an amino acid sequence corresponding to amino acid residues 19-248 of the sequence set forth in FIG. 3B (SEQ ID NO:7), to amino acid residues 19-252 of the sequence set forth in FIG. 4B (SEQ ID NO:9) or to amino acid residues 14-252 of the sequence set forth in FIG. 5B (SEQ ID NO:11). In still other specific embodiments, soluble human OSCAR polypeptides of the invention lack a transmembrane domain and (in most embodiments) a cytoplasmic tail domain. Accordingly, in still other specific embodiments an OSCAR polypeptide of the invention may comprise an amino acid sequence corresponding to: (1) amino acid residues 19-229 and, optionally, amino acid residues 249-263 of the sequence set forth in FIG. 3B (SEQ ID NO:7); (2) amino acid residues 19-233 and, optionally, amino acid residues 234-252 of the sequence set forth in FIG. 4B (SEQ ID NO:9); or (3) amino acid residues 14-218 and, optionally, amino acid residues 219-237 of the sequence set forth in FIG. 5B (SEQ ID NO:11).

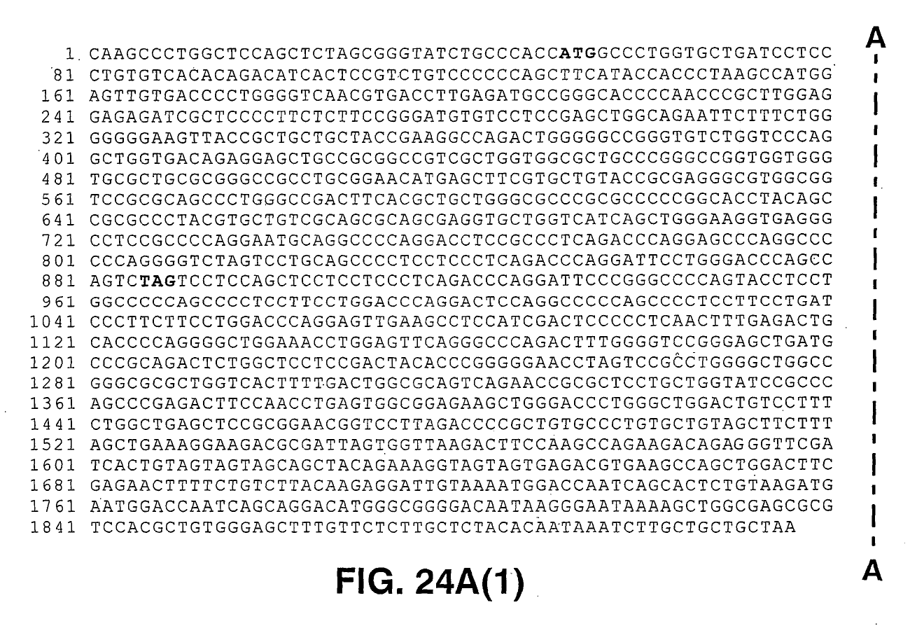

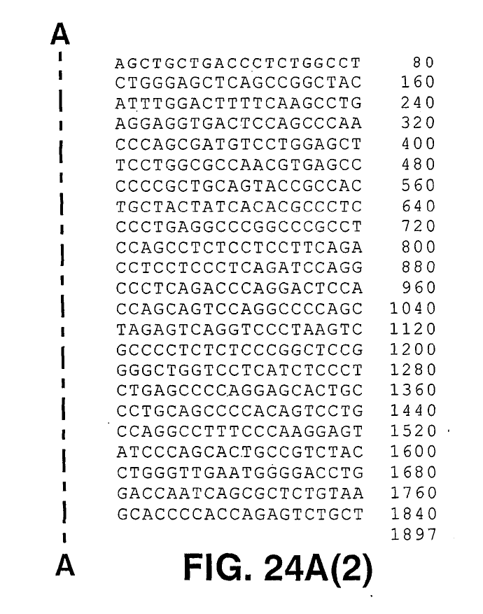

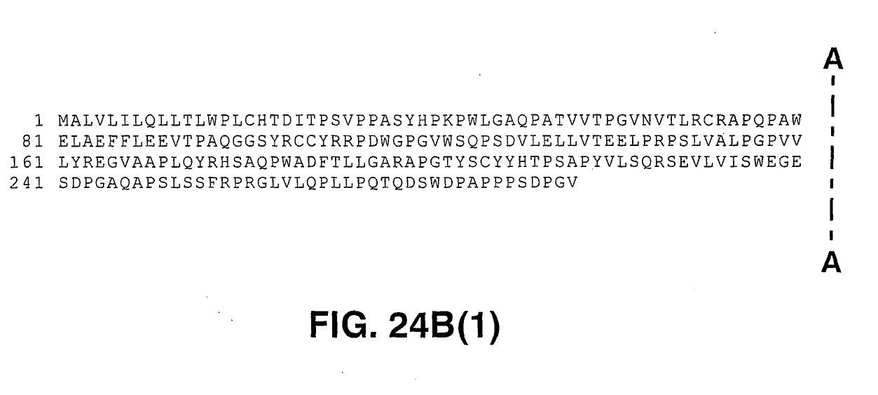

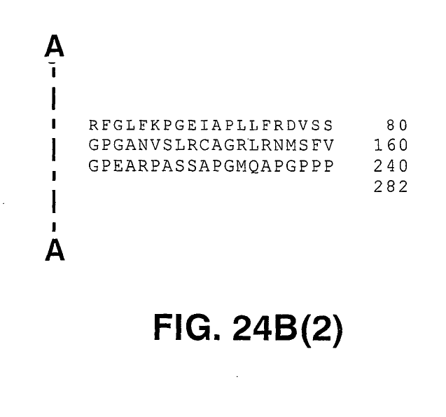

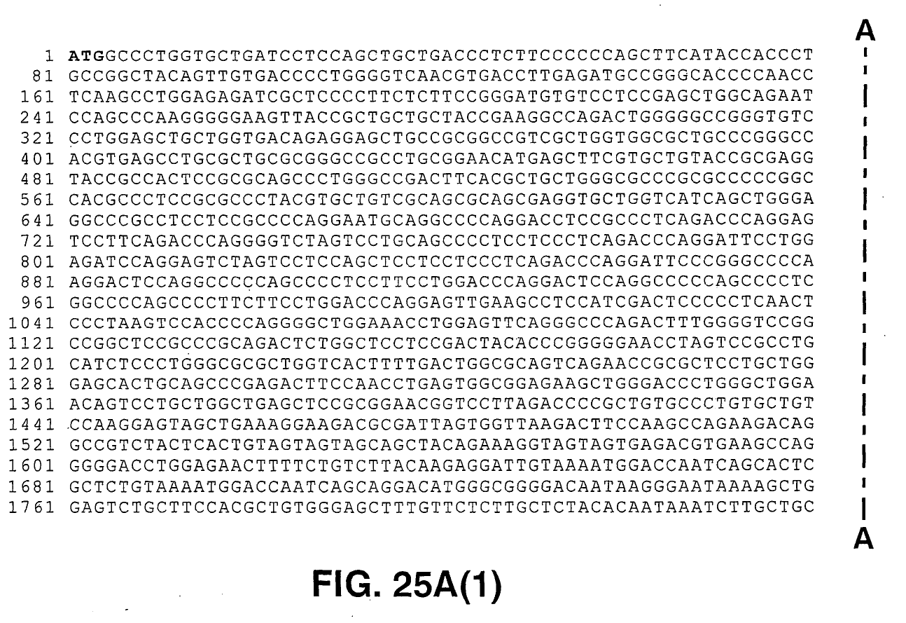

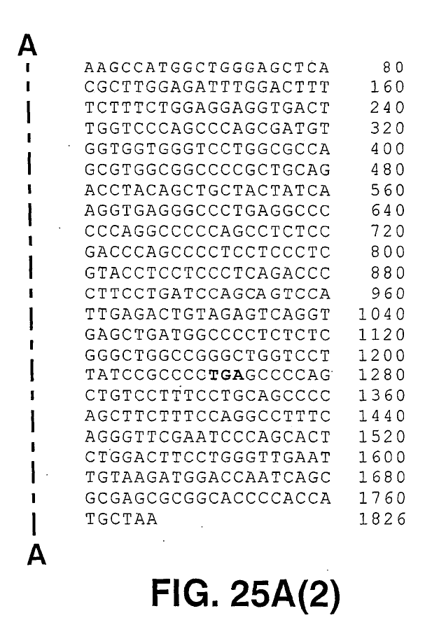

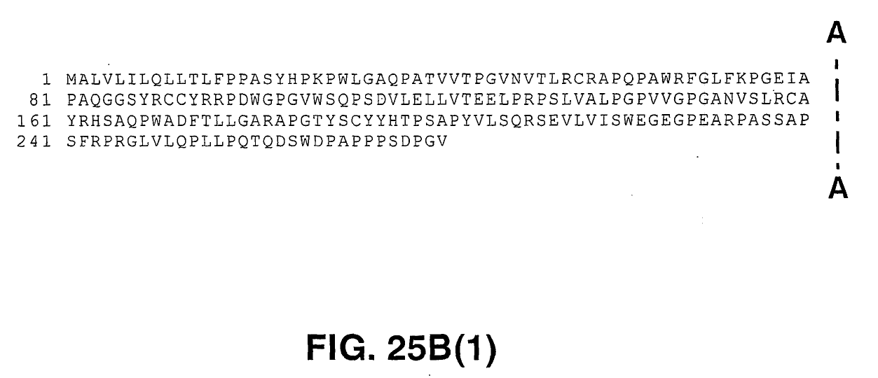

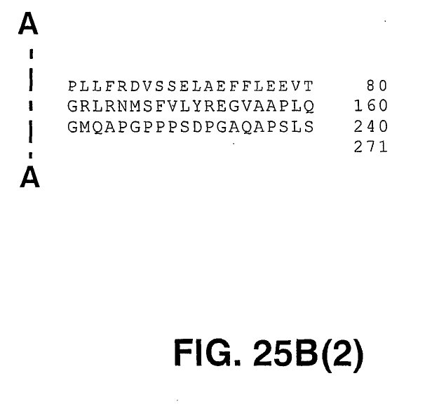

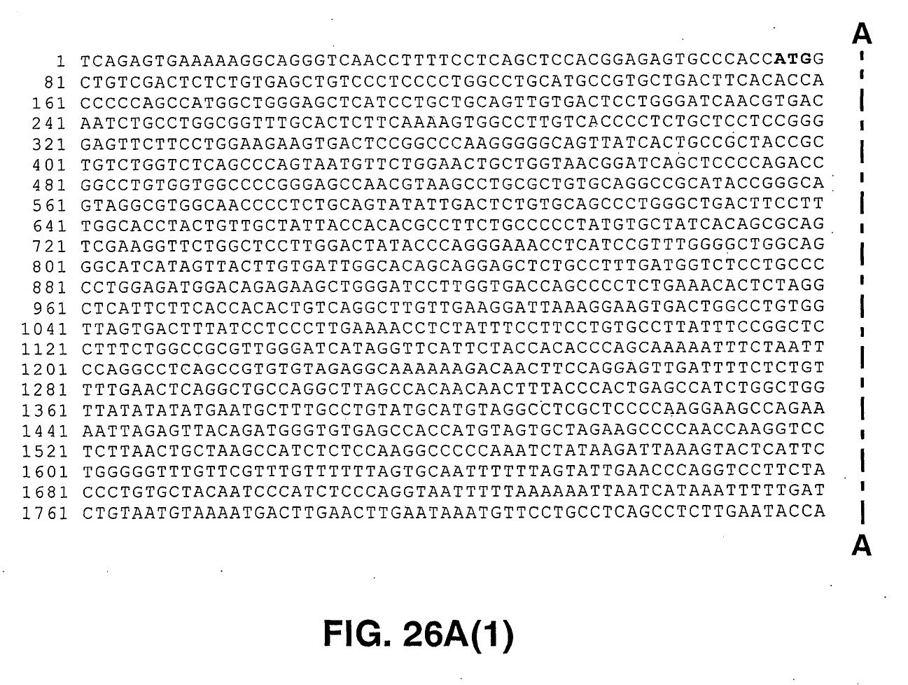

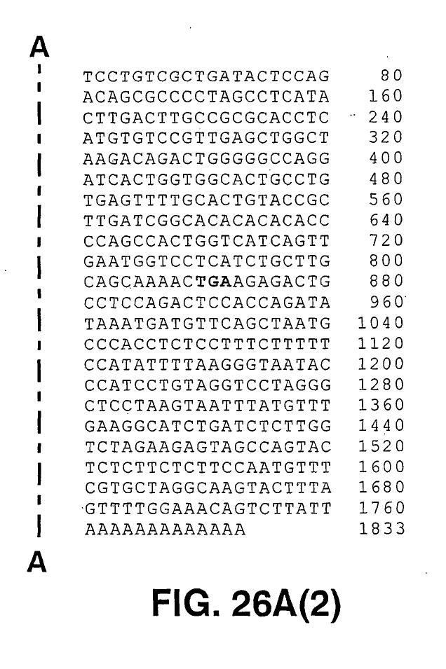

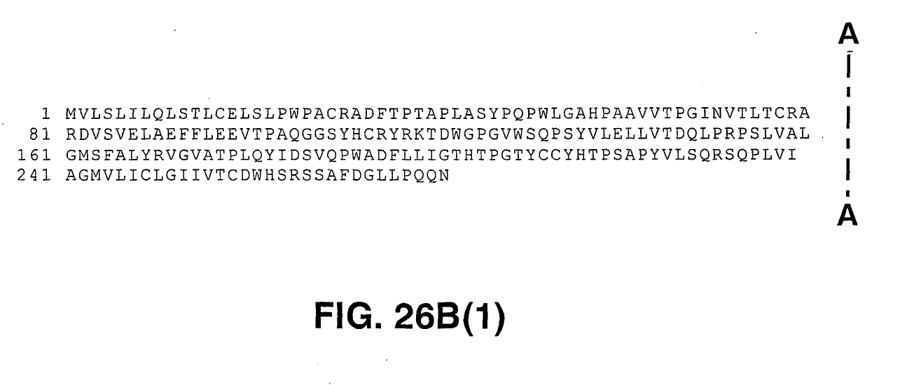

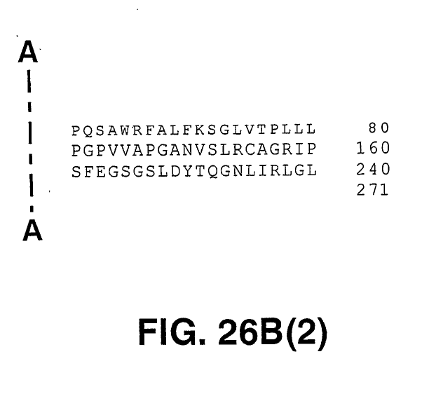

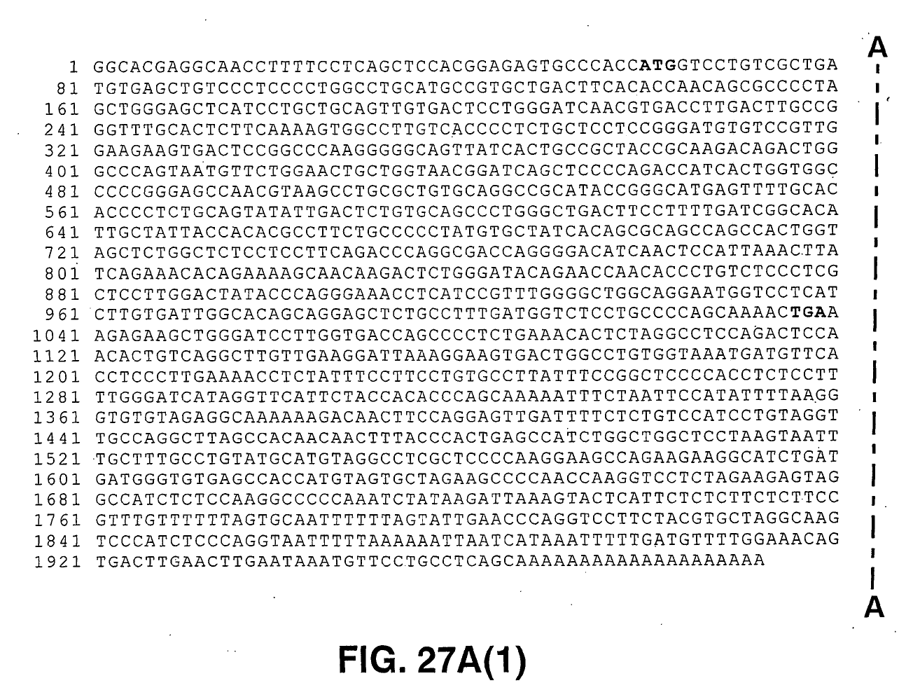

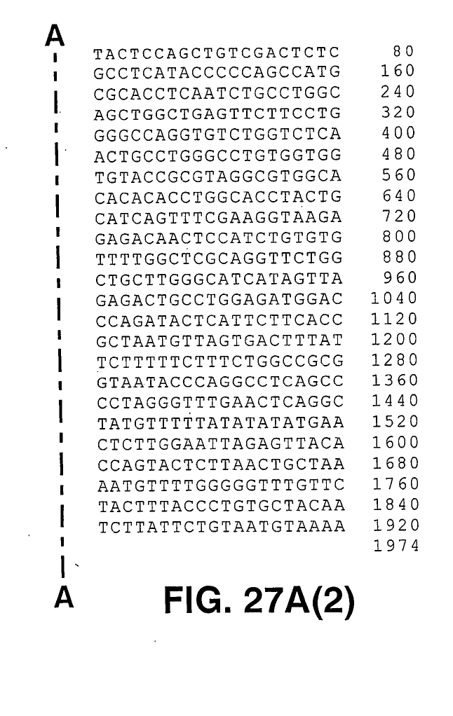

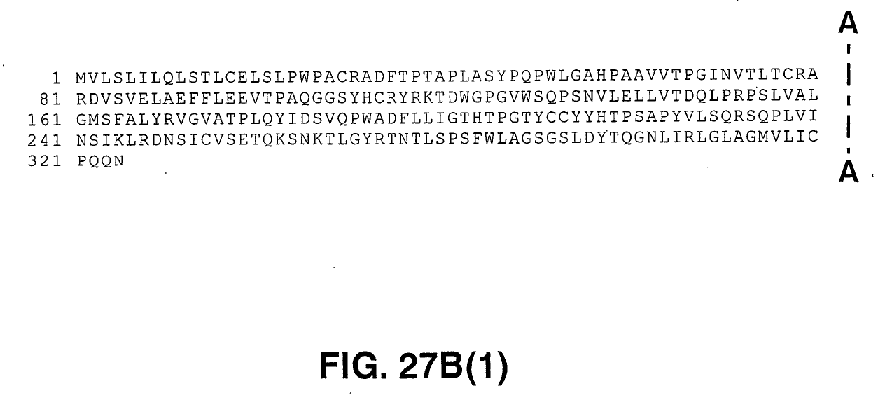

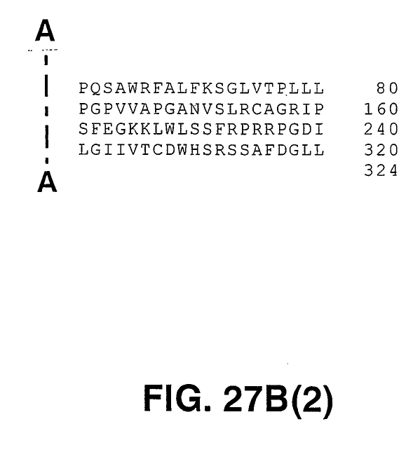

In other, alternative embodiments an OSCAR polypeptide of the invention is one which is at least 25%, or at least 30%, at least 50%, more preferably at least 70%, still more preferably at least 75% and even more preferably at least 90% identical to the OSCAR polypeptide sequence set forth in FIG. 1C (SEQ ID NO:3), in FIG. 3B (SEQ ID NO:7), in FIG. 4B (SEQ ID NO:9) in FIG. 5B (SEQ ID NO:11), in FIG. 24B (SEQ ID NO. 25), in FIG. 25B (SEQ ID NO:27), in FIG. 26B (SEQ ID NO:29) and in FIG. 27B (SEQ ID NO:31).

In still other embodiments, the OSCAR polypeptides of the invention comprise fragments of a full length OSCAR polypeptide (for example, fragments of SEQ ID NO:3, 7, 9 or 11) described herein. For instance, the Examples, infra, describe an OSCAR gene fragment (referred to as OCL178) that encodes a fragment of the full length OSCAR gene product comprising the amino acid sequence depicted in FIG. 2B (SEQ ID NO:5). Other exemplary fragments of full length OSCAR gene product are polypeptides the comprise one of the domains described above for full length OSCAR polypeptides (e.g., fragments comprising the amino acid sequence of a signal sequence domain, an Ig-like domain, a transmembrane domain, or a cyplasmic tail domain) or fragments comprising a portion of one of these domains. Other fragments of full length OSCAR polypeptides include ones which comprise any combination of two or more of the domains described above for full length OSCAR polypeptides; e.g., fragments comprising the amino acid sequence corresponding to two or more domains selected from the group consisting of a signal sequence domain, an Ig-like domain (e.g., the first or second Ig-like domain of SEQ ID NO:3, 7, 9 or 11), a transmembrane domain, or a cytoplasmic tail domain.

Such fragments of OSCAR polypeptides are useful, e.g., for constructing various fusion polypeptides as defined below. For example, fusion polypeptides that comprise a signal sequence domain can be used to target the fusion polypeptide for secretion by a host cell into the culture medium for extraction and purification. Fusion polypeptides comprising a transmembrane domain can be used to target fusion polypeptides for expression on the cell surface. In preferred embodiments, fusion polypeptides that comprise one or more Ig-like domains of a full length OSCAR polypeptide can be used to synthesize antibodies the specifically bind to the Ig-like domain and can be used to detect OSCAR expression on the surface of osteoclast cells. Alternatively, soluble fusion polypeptides comprising an OSCAR Ig-like domain can be synthesized which bind to an OSCAR ligand. Such fusion polypeptides are described in the Examples, infra and are useful, e.g., as competitors for an OSCAR ligand and to decrease the number and activity of osteoclast cells. Thus, the OSCAR polypeptides of the invention include fusion polypeptides which comprise a sequence of an OSCAR gene product or a fragment thereof.

An OSCAR nucleic acid can be a DNA or RNA molecule as well as nucleic acid molecules comprising any of the modifications (e.g., modified bases and/or backbone) described below. In one preferred embodiment, the nucleic acid has at least 50%, more preferably at least 75% and still more preferably at least 90% sequence identity to a coding sequence which encodes an OSCAR polypeptide of the invention; for example the coding sequence depicted in FIGS. 1A-B (SEQ ID NOS:1-2), or in any one of FIGS. 3A, 4A, 5A, 24A, 25A, 26A or 27A (SEQ ID NOS:6, 8, 10, 26, 28, 30 and 32 respectively). Alternatively, an OSCAR nucleic acid of the invention may be one which encodes a polypeptide that is at least 25%, more preferably at least 50%, still more preferably at least 70%, still more preferably at least 75% and even more preferably at least 90% identical to _ the OSCAR polypeptide sequence set forth, e.g., in FIG. 1C (SEQ ID NO:3), or in any on of FIGS. 3B, 4B, 5B, 24B, 25B, 26B, or 27B (SEQ ID NOS:7, 9, 11, 25,27, 29 and 31, respectively).

Alternatively, a nucleic acid encoding an OSCAR polypeptide may hybridize, under conditions set forth in detail below, to the complement of such a coding sequence or to a fragment of such a coding sequence. For instance, the Examples, infra, describe the identification of OSCAR mRNA molecules of 4.0 kb, 1.8 kb and 1.0 kb apparent length as determined by electrophoresis in agarose gels, respectively, that hybridize to the OSCAR fragment contained in the clone OCL178 and set forth in FIG. 2 (SEQ ID NO:4).

The OSCAR nucleic acids of the invention include nucleic acids, such as mRNA and cDNA derived therefrom, that have been processed or “spliced” to remove intronic sequences from an OSCAR genomic sequence. Alternatively, the OSCAR nucleic acids of the invention may be unprocessed nucleic acids, for example genomic OSCAR sequences, unspliced OSCAR mRNA sequences and cDNA sequences derived therefrom, which comprise both exon and intron sequences.

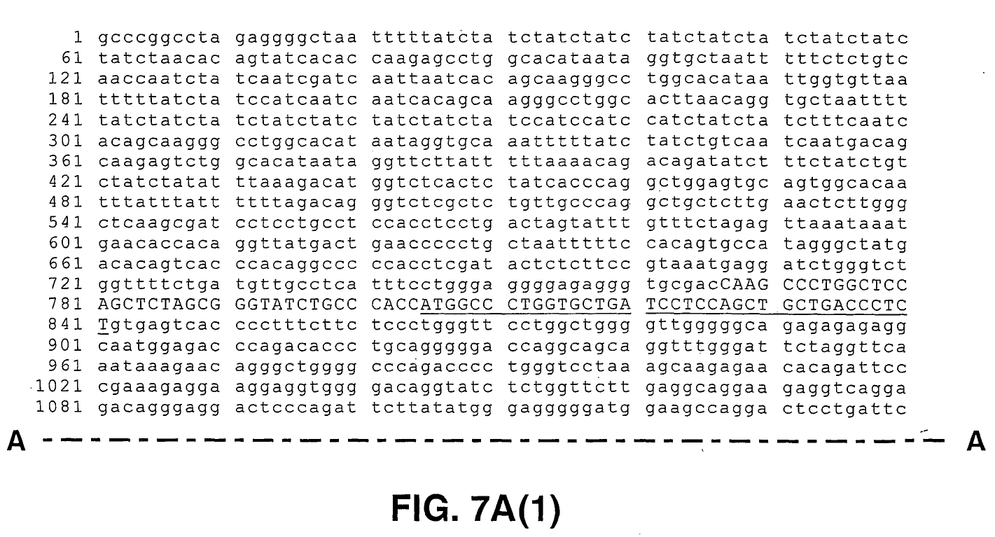

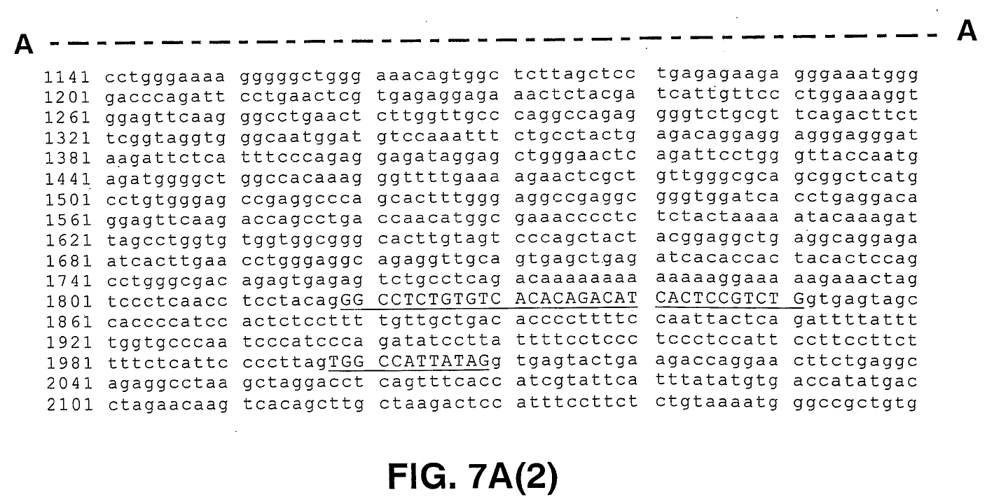

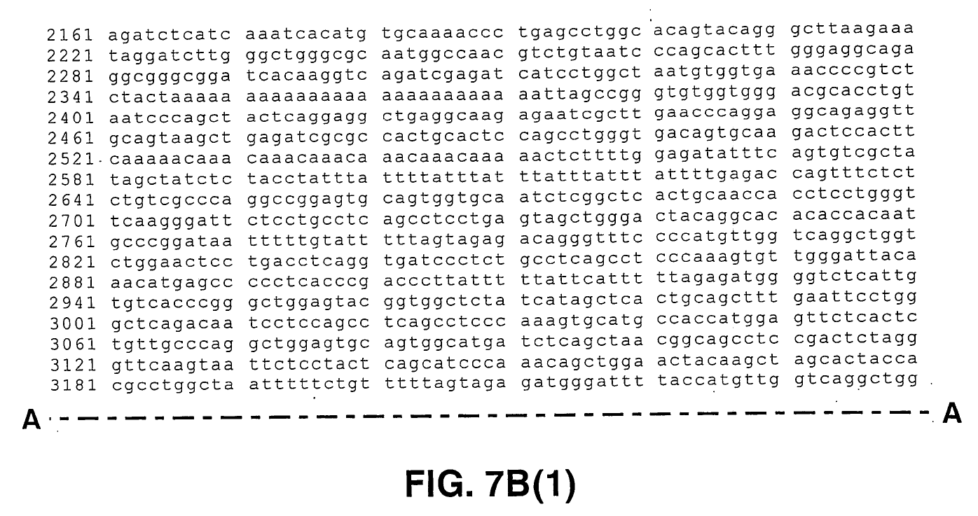

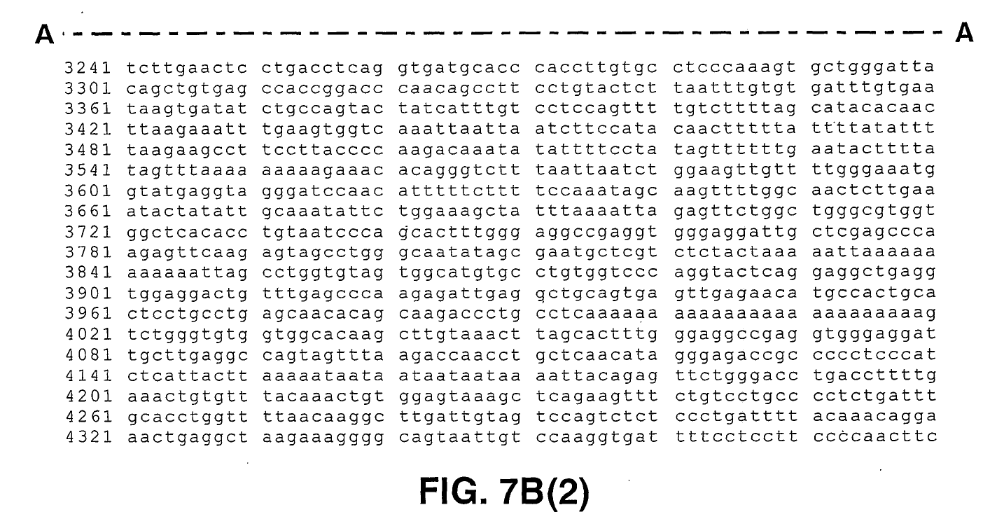

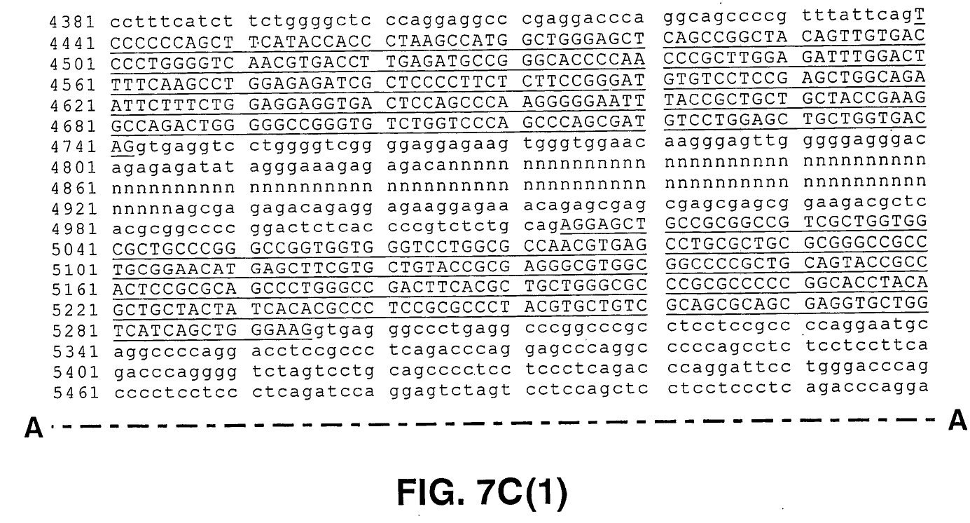

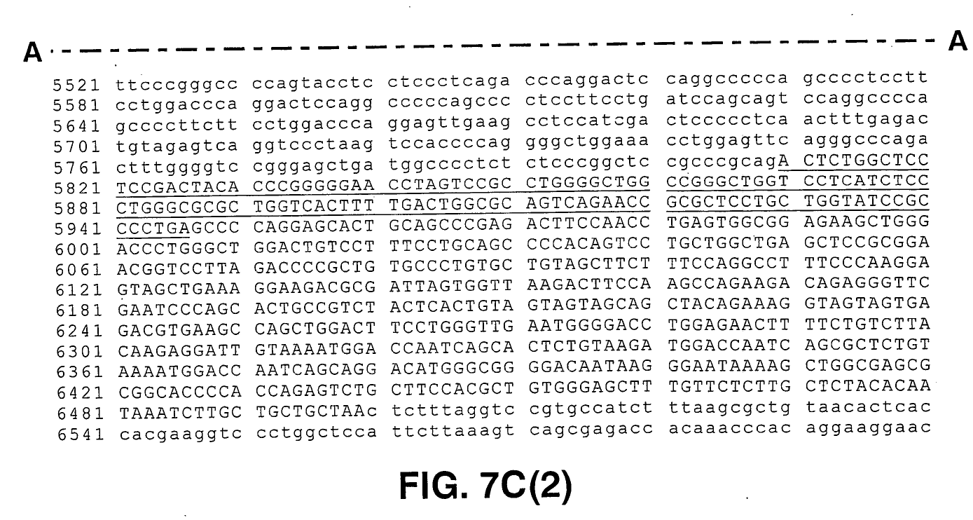

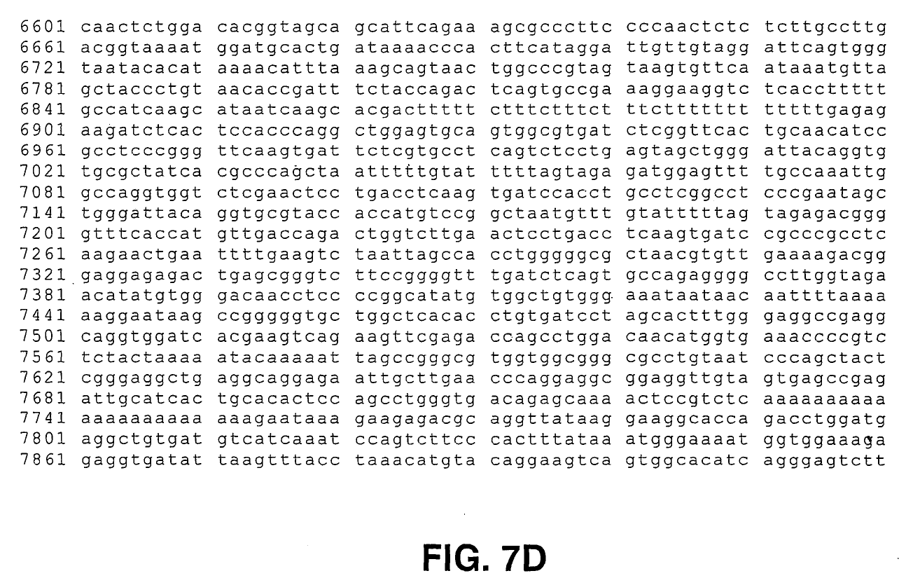

For example, FIGS. 7A-D set forth the nucleotide sequence (SEQ ID NO:12) of a region from the human chromosome 19 clone CTD-3093 (GenBank Accession No. AC012314.5; GI:771547) which contains sequences of a human OSCAR gene. The presence of such OSCAR genomic sequences with this region of human chromosome sequence was previously unknown and is described here for the first time. Such OSCAR genomic sequences are therefore among the OSCAR nucleic acids of the present invention. In particular, the genomic sequence set forth in FIGS. 7A-D (SEQ ID NO:12) includes exons sequences which are or may be transcribed into RNA encoding an OSCAR gene product of the invention. These exons sequences are indicated in FIGS. 7A-D by upper case characters. The genomic sequences set forth in FIGS. 7A-D (SEQ ID NO:12) also include intron sequences and sequences of a 5′- and 3′-unprocessed region (UPR), all of which are indicated in FIGS. 7A-D by lower case characters. Specifically, the OSCAR genomic sequence set forth in FIGS. 7A-D and in SEQ ID NO:12 includes the intron and exon domains set forth, inter alia, in TABLE 1.

TABLE 1
INTRON/EXON BOUNDARIES
OF HUMAN OSCAR (SEQ ID NO: 12)

| Nucleotide Residues | Region |
|---|---|
| 1-767 | 5′-UPR |
| 768-841 | Exon 1 |
| 842-1818 | Intron 1 |
| 1819-1851 | Exon 2 |
| 1852-1997 | Intron 2 |
| 1998-2009 | Exon 3 |
| 2010-4439 | Intron 3 |
| 4440-4742 | Exon 4 |
| 4743-5013 | Intron 4 |
| 5014-5295 | Exon 5 |
| 5296-5809 | Intron 5 |
| 5810-6499 | Exon 6 |
| 6500-7920 | 3′-UPR |

The OSCAR nucleic acid molecules of the present invention therefore include genomic OSCAR nucleic acid molecules. Such genomic OSCAR nucleic acid molecules include nucleic acids having the OSCAR genomic sequence shown in FIGS. 7A-D (SEQ ID NO:12). Genomic OSCAR nucleic acid molecules of the invention also include nucleic acid molecules having sequences which correspond to one or more exons or introns of a full length OSCAR genomic sequence, including, for example, nucleic acid sequences which correspond to one or more of the exon and intron sequences shown in FIGS. 7A-7D) and specified in TABLE 1, supra.

OSCAR nucleic acids of the invention can also contain fragments of a full length OSCAR sequence. For example, in preferred embodiments, such OSCAR nucleic acid fragments comprise a nucleotide sequence that corresponds to a sequence of at least 10 nucleotides,preferably at least 15 nucleotides and more preferably at least 20 nucleotides of a full length coding OSCAR nucleic acid sequence. In a specific embodiment, the fragments correspond to a portion (e.g, of at least 10, 15 or 20 nucleotides) of the OSCAR coding sequences depicted in any of FIGS. 1A-B, 2A, 3A, 4A, 5A, 24A, 25A, 26A, and 27A (SEQ ID NOS:1-2, 4, 6, 8, 10, 26, 28, 30 and 32 respectively). In other preferred embodiments, the OSCAR nucleic acid fragments comprise sequences of at least 10, preferably at least 15 and more preferably at least 20 nucleotides that hybridize, under conditions described in detail below, to a full length OSCAR nucleic acid sequence, for example to any of the OSCAR nucleic acid sequences depicted in FIGS. 1A-B, 2A, 3A, 4A, 5A, 24A, 25A, 26A, and 27A (SEQ ID NOS:1-2, 4, 6, 8, 10, 26, 28, 30 and 32 respectively), or to the complement of such a full length OSCAR sequence. The OSCAR nucleic acid fragments of the invention may also comprise a nucleotide sequence that corresponds to a sequence of at least 10, 15 or 20 nucleotides of an OSCAR genomic sequence (e.g., the sequence depicted in FIGS. 7A-D and set forth in SEQ ID NO:12). Alternatively, the OSCAR nucleic acid fragments may comprise sequences of at least 10, 15 or 20 nucleotides that hybridize, under conditions described in detail below, to an OSCAR genomic sequence (e.g., the genomic sequence depicted in FIGS. 7A-D and set forth in SEQ ID NO:12), to one or more exons or introns of an OSCAR genomic sequence (e.g., the exons and introns shown in FIGS. 7A-D and described in TABLE 1, supra) or to the complement of such an OSCAR genomic sequence.

Nucleic acid molecules comprising such fragments are useful, for example, as oligonucleotide probes and primers to detect or amplify an OSCAR gene. Oligonucleotide fragments can also be used, however, as antisense nucleic acids, as triple-helix forming oligonucleotides or as ribozymes. However, nucleic acid molecules of the invention that comprise one or more fragments of an OSCAR sequence can also be full length coding sequences for an OSCAR gene product.

### Definitions

General Definitions. The terms used in this specification generally have their ordinary meanings in the art, within the context of this invention and in the specific context where each term is used. Certain terms are discussed below, or elsewhere in the specification, to provide additional guidance to the practitioner in describing the devices and methods of the invention and how to make and use them.

The terms “bone growth related disorder”, “bone growth associated disorder”, “bone growth disorder”, “bone growth disease” and other such variations thereof, as generally used herein, mean any disease or disorder related to the abnormal growth, repair development, resorption, resorption, degradation or homeostasis of bone tissue. Bone growth related disorders may therefore include diseases and disorders that are associated with abnormal increases, as well as abnormal decreases of bone mass in individuals. Also, the bone growth related disorders which are the subject of the present invention may include, but are not limited to, disorders that are associated with abnormal (e.g., increased or decreased) activity of osteoclast cells. The bone growth related disorders which are the subject of the present invention further include disorders that are associated with abnormal (e.g., increased or decreased) activity of osteoblast cells. Exemplary bone growth related disorders that may be diagnosed or treated according to the methods and compositions of the present invention include osteopetrosis, osteoporosis, Paget's disease, osteogenesis imperfecta, fibrous dysplasia, hypophosphatasia, primary hyperparathyroidism arthritis, peridontal disease and myeloma blood diseases to name a few. Additionally, osteolysis can be induced by many malignant tumors resident in or distant from bone, e.g., skeletal metastases in cancers of the breast, lung, prostate, thyroid, and kidney, humoral hypercalcemia during malignancy, and multiple myelomas.

A bone growth related disorder may be associated either directly or indirectly with an OSCAR nucleic acid, gene product or polypeptide. Such disorders include ones that are associated with the abnormal synthesis or expression of an OSCAR gene or its gene product, and also diseases and disorders that are caused by an abnormal (e.g., increased or decreased) activity of an OSCAR gene and its gene product, for example disorders associated with an abnormal bioactivity of an OSCAR gene or its gene product. Other OSCAR related disorders of the invention include ones that are associated with the abnormal synthesis, expression or activity of another compound (for example a natural ligand or other cellular compound) that interacts with an OSCAR gene, an OSCAR gene product or an OSCAR polypeptide. In addition, the OSCAR related disorders the invention include ones that, while not themselves caused by or associated with abnormal synthesis, expression or activity of an OSCAR gene or gene product, can be treated by methods which modulate (e.g., increase or decrease) the synthesis, the expression or the activity of an OSCAR gene, an OSCAR gene product or an OSCAR polypeptide, or by methods which modulate the synthesis, the expression or the activity of a compound (for example a natural ligand or other cellular compound) that interacts with an OSCAR gene, gene product or polypeptide.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting [FACS]). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

A “sample” as used herein refers to a biological material which can be tested for the presence of OSCAR polypeptides or OSCAR nucleic acids, e.g., to evaluate a gene therapy or expression in a transgenic animal or to identify cells, such as osteoclasts, that specifically express the OSCAR gene and its gene product. Such samples can be obtained from any source, including tissue, blood and blood cells, including circulating hematopoietic stem cells (for possible detection of protein or nucleic acids), plural effusions, cerebrospinal fluid (CSF), ascites fluid, and cell culture. In preferred embodiments samples are obtained from bone marrow.

Non-human animals include, without limitation, laboratory animals such as mice, rats, rabbits, hamsters, guinea pigs, etc.; domestic animals such as dogs and cats; and, farm animals such as sheep, goats, pigs, horses, and cows, and especially such animals made transgenic with human or murine OSCAR.

In preferred embodiments, the terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “molecule” means any distinct or distinguishable structural unit of matter comprising one or more atoms, and includes, for example, polypeptides and polynucleotides.

Molecular Biology Definitions. In accordance with the present invention, there may be employed conventional molecular biology, microbiology and recombinant DNA techniques within the skill of the art. Such techniques are explained fully in the literature. See, for example, Sambrook, Fitsch & Maniatis, Molecular Cloning: A Laboratory Manual, Second Edition (1989) Cold Spring Harbor Laboratory Press, Cold Spring Harbor, N.Y. (referred to herein as “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins, eds. 1984); Animal Cell Culture (R. I. Freshney, ed. 1986); Immobilized Cells and Enzymes (IRL Press, 1986); B. E. Perbal, A Practical Guide to Molecular Cloning (1984); F. M. Ausubel et al. (eds.), Current Protocols in Molecular Biology, John Wiley & Sons, Inc. (1994).

The term “polymer” means any substance or compound that is composed of two or more building blocks (‘mers’) that are repetitively linked together. For example, a “dimer” is a compound in which two building blocks have been joined togther; a “trimer” is a compound in which three building blocks have been joined together; etc.

The term “polynucleotide” or “nucleic acid molecule” as used herein refers to a polymeric molecule having a backbone that supports bases capable of hydrogen bonding to typical polynucleotides, wherein the polymer backbone presents the bases in a manner to permit such hydrogen bonding in a specific fashion between the polymeric molecule and a typical polynucleotide (e.g., single-stranded DNA). Such bases are typically inosine, adenosine, guanosine, cytosine, uracil and thymidine. Polymeric molecules include “double stranded” and “single stranded” DNA and RNA, as well as backbone modifications thereof (for example, methylphosphonate linkages).

Thus, a “polynucleotide” or “nucleic acid” sequence is a series of nucleotide bases (also called “nucleotides”), generally in DNA and RNA, and means any chain of two or more nucleotides. A nucleotide sequence frequently carries genetic information, including the information used by cellular machinery to make proteins and enzymes. The terms include genomic DNA, cDNA, RNA, any synthetic and genetically manipulated polynucleotide, and both sense and antisense polynucleotides. This includes single- and double-stranded molecules; i e., DNA-DNA, DNA-RNA, and RNA-RNA hybrids as well as “protein nucleic acids” (PNA) formed by conjugating bases to an amino acid backbone. This also includes nucleic acids containing modified bases, for example, thio-uracil, thio-guanine and fluoro-uracil.

The polynucleotides herein may be flanked by natural regulatory sequences, or may be associated with heterologous sequences, including promoters, enhancers, response elements, signal sequences, polyadenylation sequences, introns, 5′- and 3′-non-coding regions and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.) and alkylators to name a few. The polynucleotides may be derivatized by formation of a methyl or ethyl phosphotriester or an alkyl phosphoramidite linkage. Furthermore, the polynucleotides herein may also be modified with a label capable of providing a detectable signal, either directly or indirectly. Exemplary labels include radioisotopes, fluorescent molecules, biotin and the like. Other non-limiting examples of modification which may be made are provided, below, in the description of the present invention.

A “polypeptide” is a chain of chemical building blocks called amino acids that are linked together by chemical bonds called “peptide bonds”. The term “protein” refers to polypeptides that contain the amino acid residues encoded by a gene or by a nucleic acid molecule (e.g., an mRNA or a cDNA) transcribed from that gene either directly or indirectly. Optionally, a protein may lack certain amino acid residues that are encoded by a gene or by an mRNA. For example, a gene or mRNA molecule may encode a sequence of amino acid residues on the N-terminus of a protein (i.e., a signal sequence) that is cleaved from, and therefore may not be part of, the final protein. A protein or polypeptide, including an enzyme, may be a “native” or “wild-type”, meaning that it occurs in nature; or it may be a “mutant”, “variant” or “modified”, meaning that it has been made, altered, derived, or is in some way different or changed from a native protein or from another mutant.

A “ligand” is, broadly speaking, any molecule that binds to another molecule. In preferred embodiments, the ligand is either a soluble molecule or the smaller of the two molecules or both. The other molecule is referred to as a “receptor”. In preferred embodiments, both a ligand and its receptor are molecules (preferably proteins or polypeptides) produced by cells. In particularly preferred embodiments, a ligand is a soluble molecule and the receptor is an integral membrane protein (i.e., a protein expressed on the surface of a cell). However, the distinction between which molecule is the ligand and which is the receptor may be an arbitrary one, such as in embodiments wherein both an OSCAR polypeptide of the invention and an OSCAR-specific ligand are or appear to be integral membrane proteins.

The binding of a ligand to its receptor is frequently a step in signal transduction within a cell. Exemplary ligand-receptor interactions include, but are not limited to, binding of a hormone to a hormone receptor (for example, the binding of estrogen to the estrogen receptor) and the binding of a neurotransmitter to a receptor on the surface of a neuron.

“Amplification” of a polynucleotide, as used herein, denotes the use of polymerase chain reaction (PCR) to increase the concentration of a particular DNA sequence within a mixture of DNA sequences. For a description of PCR see Saiki et al., Science 1988, 239:487.

“Chemical sequencing” of DNA denotes methods such as that of Maxam and Gilbert (Maxam-Gilbert sequencing; see Maxam & Gilbert, Proc. Natl. Acad. Sci. U.S.A. 1977, 74:560), in which DNA is cleaved using individual base-specific reactions.

“Enzymatic sequencing” of DNA denotes methods such as that of Sanger (Sanger et al., Proc. Natl. Acad. Sci. U.S.A. 1977, 74:5463) and variations thereof well known in the art, in a single-stranded DNA is copied and randomly terminated using DNA polymerase.

A “gene” is a sequence of nucleotides which code for a functional “gene product”. Generally, a gene product is a functional protein. However, a gene product can also be another type of molecule in a cell, such as an RNA (e.g., a tRNA or a rRNA). For the purposes of the present invention, a gene product also refers to an mRNA sequence which may be found in a cell. For example, measuring gene expression levels according to the invention may correspond to measuring mRNA levels. A gene may also comprise regulatory (i.e., non-coding) sequences as well as coding sequences. Exemplary regulatory sequences include promoter sequences, which determine, for example, the conditions under which the gene is expressed. The transcribed region of the gene may also include untranslated regions including introns, a 5′-untranslated region (5′-UTR) and a 3′-untranslated region (3′-UTR).

A “coding sequence” or a sequence “encoding” and expression product, such as a RNA, polypeptide, protein or enzyme, is a nucleotide sequence that, when expressed, results in the production of that RNA, polypeptide, protein or enzyme; i.e., the nucleotide sequence “encodes” that RNA or it encodes the amino acid sequence for that polypeptide, protein or enzyme.

A “promoter sequence” is a DNA regulatory region capable of binding RNA polymerase in a cell and initiation transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently found, for example, by mapping with nuclease S1), as well as protein binding domains (consensus sequences) responsible for the binding of RNA polymerase.

A coding sequence is “under the control of” or is “operatively associated with” transcriptional and translational control sequences in a cell when RNA polymerase transcribes the coding sequence into RNA, which is then trans-RNA spliced (if it contains introns) and, if the sequence encodes a protein, is translated into that protein.

The term “express” and “expression” means allowing or causing the information in a gene or DNA sequence to become manifest, for example producing RNA (such as rRNA or mRNA) or a protein by activating the cellular functions involved in transcription and translation of a corresponding gene or DNA sequence. A DNA sequence is expressed by a cell to form an “expression product” such as an RNA (e.g., a mRNA or a rRNA) or a protein. The expression product itself, e.g., the resulting RNA or protein, may also said to be “expressed” by the cell.

The term “transfection” means the introduction of a foreign nucleic acid into a cell. The term “transformation” means the introduction of a “foreign” (i.e., extrinsic or extracellular) gene, DNA or RNA sequence into a host cell so that the host cell will express the introduced gene or sequence to produce a desired substance, in this invention typically an RNA coded by the introduced gene or sequence, but also a protein or an enzyme coded by the introduced gene or sequence. The introduced gene or sequence may also be called a “cloned” or “foreign” gene or sequence, may include regulatory or control sequences (e.g., start, stop, promoter, signal, secretion or other sequences used by a cell's genetic machinery). The gene or sequence may include nonfunctional sequences or sequences with no known function. A host cell that receives and expresses introduced DNA or RNA has been “transformed” and is a “transformant” or a “clone”. The DNA or RNA introduced to a host cell can come from any source, including cells of the same genus or species as the host cell or cells of a different genus or species.

The terms “vector”, “cloning vector” and “expression vector” mean the vehicle by which a DNA or RNA sequence (e.g., a foreign gene) can be introduced into a host cell so as to transform the host and promote expression (e.g., transcription and translation) of the introduced sequence. Vectors may include plasmids, phages, viruses, etc. and are discussed in greater detail below.

A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A large number of vectors, including plasmid and fungal vectors, have been described for replication and/or expression in a variety of eukaryotic and prokaryotic hosts. The term “host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells can further be used for screening or other assays that are described infra. Host cells may be cultured in vitro or one or more cells in a non-human animal (e.g., a transgenic animal or a transiently transfected animal).

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells such as Sf9, Hi5 or S2 cells and Baculovirus vectors, Drosophila cells (Schneider cells) and expression systems, and mammalian host cells and vectors. For example, OSCAR may be expressed in PC12, COS-1, or C₂C₁₂ cells. Other suitable cells include CHO cells, HeLa cells, 293T (human kidney cells), mouse primary myoblasts, and NIH 3T3 cells.

The term “heterologous” refers to a combination of elements not naturally occurring. For example, the present invention includes chimeric RNA molecules that comprise an rRNA sequence and a heterologous RNA sequence which is not part of the rRNA sequence. In this context, the heterologous RNA sequence refers to an RNA sequence that is not naturally located within the ribosomal RNA sequence. Alternatively, the heterologous RNA sequence may be naturally located within the ribosomal RNA sequence, but is found at a location in the rRNA sequence where it does not naturally occur. As another example, heterologous DNA refers to DNA that is not naturally located in the cell, or in a chromosomal site of the cell. Preferably, heterologous DNA includes a gene foreign to the cell. A heterologous expression regulatory element is a regulatory element operatively associated with a different gene that the one it is operatively associated with in nature.

The terms “mutant” and “mutation” mean any detectable change in genetic material, e.g., DNA, or any process, mechanism or result of such a change. This includes gene mutations, in which the structure (e.g., DNA sequence) of a gene is altered, any gene or DNA arising from any mutation process, and any expression product (e.g., RNA, protein or enzyme) expressed by a modified gene or DNA sequence. The term “variant” may also be used to indicate a modified or altered gene, DNA sequence, RNA, enzyme, cell, etc.; i.e., any kind of mutant. For example, the present invention relates to altered or “chimeric” RNA molecules that comprise an rRNA sequence that is altered by inserting a heterologous RNA sequence that is not naturally part of that sequence or is not naturally located at the position of that rRNA sequence. Such chimeric RNA sequences, as well as DNA and genes that encode them, are also referred to herein as “mutant” sequences.

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin or a fluorescent dye (for example, Cy3 or Cy5) has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of OSCAR, or to detect the presence of nucleic acids encoding OSCAR. In a further embodiment, an oligonucleotide of the invention can form a triple helix with an OSCAR DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

The present invention provides antisense nucleic acids (including ribozymes), which may be used to inhibit expression of an OSCAR gene or its gene product. An “antisense nucleic acid” is a single stranded nucleic acid molecule which, on hybridizing under cytoplasmic conditions with complementary bases in an RNA or DNA molecule, inhibits the latter's role. If the RNA is a messenger RNA transcript, the antisense nucleic acid is a countertranscript or mRNA-interfering complementary nucleic acid. As presently used, “antisense” broadly includes RNA-RNA interactions, RNA-DNA interactions, triple helix interactions, ribozymes and RNase-H mediated arrest. Antisense nucleic acid molecules can be encoded by a recombinant gene for expression in a cell (e.g., U.S. Pat. No. 5,814,500; U.S. Pat. No. 5,811,234), or alternatively they can be prepared synthetically (e.g., U.S. Pat. No. 5,780,607). Other specific examples of antisense nucleic acid molecules of the invention are provided infra.

Specific non-limiting examples of synthetic oligonucleotides envisioned for this invention include, in addition to the nucleic acid moieties described above, oligonucleotides that contain phosphorothioates, phosphotriesters, methyl phosphonates, short chain alkyl, or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—PO₂—O—CH₂). U.S. Pat. No. 5,677,437 describes heteroaromatic olignucleoside linkages. Nitrogen linkers or groups containing nitrogen can also be used to prepare oligonucleotide mimics (U.S. Pat. Nos. 5,792,844 and 5,783,682). U.S. Pat. No. 5,637,684 describes phosphoramidate and phosphorothioamidate oligomeric compounds. Also envisioned are oligonucleotides having morpholino backbone structures (U.S. Pat. No. 5,034,506). In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al., Science 254:1497, 1991). Other synthetic oligonucleotides may contain substituted sugar moieties comprising one of the following at the 2′ position: OH, SH, SCH₃, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O—; S—, or N-alkyl; O—, S—, or N-alkenyl; SOCH₃ ; SO₂CH₃; ONO₂;NO₂; N₃; NH₂; heterocycloalkyl; heterocycloalkaryl; aminoalkylamino; polyalkylamino; substitued silyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties. Oligonucleotides may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ (melting temperature) of 55 ° C., can be used, e.g., 5×SSC, 0.1% SDS, 0.25% milk, and no formamide; or 30% formarmide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. SCC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% foimamide, 4×SSC, or under conditions that afford levels of hybridization equivalent to those observed under either of these two conditions.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligoncucleotide molecules used. Exemplary temperatures may be 37° C. (for 14-base oligonucleotides), 48° C. (for 17-base oligoncucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

### OSCAR Polypeptides

OSCAR polypeptides of the present invention are defined above. One preferred OSCAR polypeptide comprises a sequence of about 264 amino acid residues in length and preferably includes a signal peptide sequence that is about 16 amino acid residues in length. In another embodiment an OSCAR polypeptide of the invention may comprise a sequence of about 248 amino acid residues in length and does not include a signal peptide sequence. The polypeptides of the two embodiments may have predicted molecular weights (calculated from their amino acid sequences) of about 28.7 kDa and about 27.0 kDa, respectively. In still other embodiment, an OSCAR polypeptide of the invention may be modified, e.g., by glycosylation. In such embodiments, the apparent molecular weight of an OSCAR polypeptide may be different from the molecular weight calculated by its amino acid sequence alone. For example, in preferred embodiments an OSCAR polypeptide may have an apparent molecular weight (determined, for example, by SDS-PAGE) of 35 kDa or 40 kDa.

As noted above, the OSCAR polypeptides of the invention can also be characterized by their expression pattern in osteoclast cells. In particular, the OSCAR genes and gene products of the invention are preferably expressed only in osteoclast cells; with the exception of host cells that have been manipulated, e.g., according to the methods described below, to express OSCAR polypeptides. In particular, the OSCAR polypeptides of the present invention preferably are not expressed in other bone marrow derived cells, including macrophages and dendritic cells. In addition, the OSCAR polypeptides of the invention preferably are not expressed in other cells or tissues of an organism, including but not limited to muscle, kidney, brain, heart, liver, intestine, thymus, spleen and lymphocyte. It is understood, however, that OSCAR polypeptides of the invention may be expressed by these and other cell types where such cells are transformed, e.g., with a vector that contains a nucleotide sequence encoding an OSCAR polypeptide.

The OSCAR polypeptides of the invention may also be characterized by their specific bioactivity. In particular, these polypeptides can modulate the maturation and activity of osteoclast cells, as demonstrated in the Examples, infra. For example, the administration of OSCAR polypeptides of the invention can decrease the maturation and activity of osteoclast cells (as determined, for example, by decreased numbers of multinucleated osteoclast cells) in the presence of osteoblast cells. While not wishing to be bound to any particular theory or mechanism of action, it is believed that such polypeptides competitively bind to an OSCAR ligand produced by the osteoblast cells. Such an OSCAR ligand will ordinarily bind to an OSCAR polypeptide expressed by osteoclast cells so that maturation of those osteoclast cells is induced. Thus, by competitively binding to the OSCAR ligand, the administration of the additional OSCAR polypeptide actually prevents the ligand's stimulation of osteoclast cells.

Alternatively, the OSCAR polypeptides of the invention, when expressed by osteoclast cells, can also be characterized by their ability to increase osteoclast maturation and/or osteoclast activity upon binding with an OSCAR ligand.

In one specific embodiment, an OSCAR polypeptide of the invention comprises the amino acid sequence set forth in FIG. 1C (SEQ ID NO:3). This murine OSCAR polypeptide comprises sequences corresponding to at least five distinct domains: a signal peptide sequence (comprising amino acid residues 1-16 of SEQ ID NO:3); two Ig-like domain sequences (comprising amino acid residues 17-122 and 123-228, respectively, on SEQ ID NO:3); a transmembrane domain sequence (comprising amino acid residues 229-247 of SEQ ID NO:3); and a cytoplasmic tail domain sequence (comprising amino acid residues (248-264 of SEQ ID NO:3). It is understood that the amino acid residue numbers specified for delineating each of these domains are approximate.

In another specific embodiment, an OSCAR polypeptide of the invention comprises the amino acid sequence of a human OSCAR polypeptide. In particular, the present invention provides at least five isoforms (i.e., variants) of a human OSCAR polypeptide. These variants are referred to herein as the C18 human OSCAR isoform (set forth in FIG. 3B and in SEQ ID NO:7), the C16 human OSCAR isoform (set forth in FIG. 4B and in SEQ ID NO:9), the C10 human OSCAR isoform (set forth in FIG. 5B and in SEQ ID NO:11), human OSCAR isoform S1 (set forth in FIG. 24B and in SEQ ID NO: 25) and human OSCAR isoform S2 (set forth in FIG. 25B and in SEQ ID NO: 27), respectively.

Thus, in one particular embodiment an OSCAR polypeptide of the invention comprises the amino acid sequence set forth in FIG. 3B (SEQ ID NO:7). This C18 human OSCAR isoform comprises sequences corresponding to at least five distinct domains: a signal peptide sequence (comprising amino acid residues 1-18 of SEQ ID NO:7), two Ig-like domain sequences (comprising amino acid residues 19-123 and 124-229, respectively, of SEQ ID NO:7), a transmembrane domain sequence (comprising amino acid residues 230-248 of SEQ ID NO:7) and a cytoplasmic tail domain sequence (comprising amino acid residues 249-263 of SEQ ID NO:7).

In another particular embodiment, an OSCAR polypeptide of the invention comprises the amino acid sequence set forth in FIG. 4B (SEQ ID NO:9). This C16 human OSCAR isoform also comprises sequences corresponding to at least five distinct domains: a signal peptide sequence (comprising amino acid residues 1-18 of SEQ ID NO:9), two Ig-like domain sequences (comprising amino acid residues 19-127 and 128-233 of SEQ ID NO:9), a transmembrane domain sequence (comprising amino acid residues 234-252 of SEQ ID NO:9) and a cytoplasmic tail domain sequence (comprising amino acid residues 253-267 of SEQ ID NO:9).

In yet another particular embodiment, an OSCAR polypeptide of the invention comprises the amino acid sequence set forth in FIG. 5B (SEQ ID NO:11). The C10 human OSCAR isoform also comprises sequences corresponding to at least five distinct domains: a signal peptide sequence (comprising amino acid residues 1-13 of SEQ ID NO:11), two Ig-like domain sequences (comprising amino acid residues 14-112 and 113-218 of SEQ ID NO:11), a transmembrane domain sequence (comprising amino acid residues 219-237 of SEQ ID NO:11) and a cytoplasmic tail domain sequence (comprising amino acid residues 238-252 of SEQ ID NO:11).

In yet another particular embodiment, an OSCAR polypeptide of the invention comprises the amino acid sequence set forth in FIG. 24B (SEQ ID NO: 25). This embodiment lacks the transmembrane domain found in the above-described embodiments.

In yet another particular embodiment, an OSCAR polypeptide of the invention comprises the amino acid sequence set forth in FIG. 25B (SEQ ID NO: 27). This embodiment alsolacks the transmembrane domain found in the above-described embodiments.

In other embodiments, an OSCAR polypeptide of the invention comprises the amino acid sequence of one or more individual domains of a full length OSCAR polypeptide such as the full length OSCAR polypeptides set forth in FIGS. 1C, 3B, 4B 5B, 24B, 25B, 26B and 27B (SEQ ID NOS:3, 7, 9, 11, 25, 27, 29 and 31 respectively). Thus, for example, OSCAR polypeptides of the invention include polypeptides having an amino acid sequence corresponding to the signal sequence domain, either Ig-like domain, the transmembrane domain or the cytoplasmic domain described above for any of the OSCAR polypeptides set forth in SEQ ID NOS:3, 7, 9 and 11. OSCAR polypeptides of the invention further include polypeptides having amino acid sequences corresponding to any combination of these individual domains. It is understood that the amino acid residue numbers specified for delineating each of these domains are approximate.

OSCAR polypeptides of the invention also include polypeptides comprising an amino acid sequence of an epitope of a full length OSCAR polypeptide, such as epitopes of any of the full length OSCAR polypeptides set forth in FIGS. 1C, 3B, 4B, 5B, 24B, 25B, 26B and 27B (SEQ ID NOS:3, 7, 9, 11, 25, 27, 29 and 31 respectively). An epitope of an OSCAR polypeptide represents a site on the polypeptide against which an antibody may be produced and to which the antibody binds. Thus, polypeptides comprising the amino acid sequence of an OSCAR epitope are useful for making antibodies to an OSCAR protein. Preferably, an epitope comprises a sequence of at least 5, more preferably at least 10, 15, 20, 25,or 50 amino acid residues in length. Thus, OSCAR polypeptides of the invention that comprise epitopes of an OSCAR protein preferably contain an amino acid sequence corresponding to a sequence of at least 5, at least 10, at least 15, at least 20, at least 25 or at least 50 amino acid residues of the OSCAR protein sequence. For example, in certain preferred embodiments wherein the epitope is an epitope of one of the OSCAR polypeptides set forth in FIGS. 1C, 3B, 4B, 5B, 24B, 25B, 26B and 27B (SEQ ID NOS:3, 7, 9, 11, 25, 27, 29 and 31 respectively), an OSCAR polypeptide comprises an amino acid sequence corresponding to a sequence of at least 5, at least 10, at least 15, at least 20, at least 25 or at least 50 amino acid residues of sequence set forth in FIGS. 1C, 3B, 4B, 5B, 24B, 25B, 26B and 27B (SEQ ID NOS:3, 7, 9, 11, 25, 27, 29 and 31 respectively).

Still other fragments are provided herein that are among the OSCAR polypeptides of the invention. For instance, the Examples, infra, provide a clone, referred to as OCL1 78, that encodes the polypeptide sequence set forth in FIG. 2B (SEQ ID NO:5). This polypeptide corresponds to the sequence of amino acid residues 161-265 of the full length polypeptide set forth in FIG. 1C (SEQ ID NO:3). Such fragments are also among the OSCAR polypeptides of the invention.

The OSCAR polypeptides of the invention also include analogs and derivatives of the full length OSCAR polypeptides (e.g., of SEQ ID NO:3, 7, 9, 11, 25 and 27). Analogs and derivatives of the OSCAR polypeptides of the invention have the same or homologous characteristics of OSCAR polypeptides set forth above.

For example, truncated forms of an OSCAR polypeptide can be provided. Such truncated forms may include an OSCAR polypeptide with a specific deletion. For instance, in certain embodiments amino acid residues corresponding to one or more domains of a full length OSCAR polypeptide (e.g., a signal sequence domain, one or more Ig-like domains, a transmembrane domain or a cytoplasmic tail domain) may be deleted from the amino acid sequence of an OSCAR polypeptide. In preferred embodiments, a truncated OSCAR polypeptide of the invention is one wherein a signal-sequence domain has been deleted or otherwise removed; i.e., one which does not comprise a signal-sequence domain.

In certain embodiments, a derivative is functionally active; i.e., it is capable of exhibiting one or more functional activities associated with a full-length, wild-type OSCAR polypeptide of the invention.

An OSCAR chimeric fusion polypeptide can be prepared in which the OSCAR portion of the fusion protein has one or more characteristics of the OSCAR polypeptide. Such fusion polypeptides therefore represent embodiments of the OSCAR polypeptides of the present invention. Exemplary OSCAR fusion polypeptides include ones which comprise a full length, derivative or truncated OSCAR amino acid sequence, as well as fusions which comprise a fragment of an OSCAR polypeptide sequence (e.g., a fragment corresponding to an epitope or to one or more domains). Such fusion polypeptides may also comprise the amino acid sequence of a marker polypeptide; for example FLAG, a histidine tag, glutathione S-transferase (GST), hemaglutinin, or Fc portion of human IgG. In other embodiments, an OSCAR polypeptide may be expressed with a bacterial protein such as β-galactosidase. Additionally, OSCAR fusion polypeptides may comprise amino acid sequences that increase solubility of the polypeptide, such as a thioreductase amino acid sequence or the sequence of one or more immunoglobulin proteins (e.g., IgG1 or IgG2).

OSCAR analogs or variants can also be made by altering encoding nucleic acid molecules, for example by substitutions, additions or deletions. Preferably such altered nucleic acid molecules encode functionally similar molecules (i.e., molecules that perform one or more OSCAR functions or have one or more OSCAR bioactivities). Thus, in a specific embodiment, an analog of an OSCAR polypeptide is a function-conservative variant.

“Function-conservative variants” of a polypeptide are those variants in which a given amino acid residue in the polypeptide has been changed without altering the overall conformation and/or function (e.g., bioactivity) of the polypeptide. Such changes include, but are not limited to, replacement of an amino acid with one having similar properties; such as similar properties of polarity, hydrogen bonding potential, acidity, alkalinity, hydrophobicity, aromaticity and the like. For example and not by way of limitation, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide.

Amino acid residues, other than ones that are specifically identified herein as being conserved, may differ among variants of a protein or polypeptide. Accordingly, the percentage of protein or amino acid sequence similarity between any two OSCAR polypeptides of similar function may vary. Typically, the percentage of protein or amino acid sequence similarity between different OSCAR polypeptide variants may be from 70% to 99%, as determined according to an alignment scheme such as the Cluster Method and/or the MEGALIGN algorithm. “Function-conservative variants” also include polypeptides that have at least 50%, preferably at least 75%, more preferably at least 85%, and still more preferably at least 90% amino acid identity as determined, e.g., by BLAST or FASTA algorithms. Preferably, such function-conservative variants also have the same or similar properties, functions or bioactivities as the native polypeptide to which they are compared. It is further noted that function-conservative variants of the present invention include, not only variants of the full length OSCAR proteins of the invention (e.g., variants of an OSCAR polypeptide comprising the sequence set forth in FIGS. 1C, 3B, 4B, 5B, 24B, 25B, 26B and 27B (SEQ ID NOS:3, 7, 9, 11, 25, 27, 29 and 31 respectively), but also include function-conservative variants of modified OSCAR polypeptides (e.g., truncations and deletions) and of fragments (e.g., corresponding to domains or epitopes) of full length OSCAR proteins.

In yet other embodiments, an analog of an OSCAR polypeptide is an allelic variant or mutant of an OSCAR polypeptide. The term allelic variant and mutant, when used to describe a polypeptide, refers to a polypeptide encoded by an allelic variant or mutant gene. Thus, the allelic variant and mutant OSCAR polypeptides of the invention are polypeptides encoded by allelic variants or mutants of the OSCAR nucleic acid molecules of the present invention.

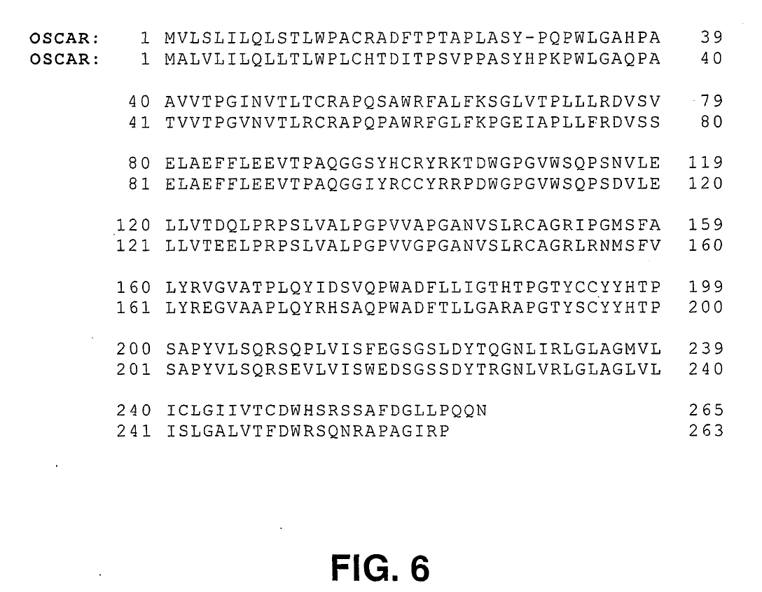

In yet other embodiments, an analog of an OSCAR polypeptide is a substantially homologous polypeptide from the same species (e.g., allelic variants) or from another species (e.g., an orthologous polypeptide); preferably from another mammalian species such as mouse, human, rat, rabbit, hamster or guinea pig. OSCAR homologs of the invention may, however, be from any species including dogs, cats, sheep, goats, pigs, horses, cows, chickens and xenopus to name a few. For example, the OSCAR polypeptide sequence set forth in FIG. 3B (SEQ ID NO:7) is a human OSCAR ortholog and is homologous to the murine OSCAR polypeptide set froth in FIG. 1C (SEQ ID NO:3). An alignment of these two amino acid sequences, which is shown in FIG. 6, demonstrates that the two sequences share considerable sequence identity. In particular, the polypeptide sequence for the C18 human OSCAR isoform (hOSCAR in FIG. 6, SEQ ID NO:7) is 74.6% (i.e., about 75%) identical to the murine OSCAR polypeptide sequence (mOSCAR in FIG. 6, SEQ ID NO:3).

As used here, the term “homologous”, in all its grammatical forms and spelling variations, refers to the relationship between proteins that are understood to possess a “common evolutionary origin”, including proteins from superfamilies (e.g., the immunoglobulin superfamily) and homologous proteins from different species. See, for example, Reeck et al., Cell 1987, 50:667. Corresponding proteins from different species are referred to as “orthologs”. Homologous and orthologous proteins, and their encoding genes, have sequence homology, as reflected by the sequence similarity. Such sequence similarity may be indicated, for example, by the percent of sequence similarity (e.g.. a percentage of amino acid sequence identity or homology), or by the presence of specific amino acid residues or motifs at conserved positions.

The terms “sequence similarity”, in all its grammatical forms, refers to the degree of identity or correspondence between nucleic acid or amino acid sequences. Except as otherwise noted herein, the term “homologous” refers merely to sequence similarity and does not necessarily relate to a common evolutionary origin.

In a specific embodiment, two polypeptide sequences are “substantially homologous” or “substantially similar” when the polypeptides are at least 3540% similar as determined by one of the algorithms disclosed herein, preferably at least about 60% and most preferably at least about 90 or 95% in one or more highly conserved domains or, for alleles, across the entire amino acid sequence. Sequence comparison algorithms that can be used to compare amino acid or nucleic acid sequences include the BLAST algorithms (e.g., BLAST P, BLAST N, BLAST X), FASTA, DNA Strider, the GCG (Genetics Computer Group, Program Manual for the GCG Pakcage, Version 7, Madison, Wis.) pileup program, etc. Unless otherwise stated, all sequence comparisons referred to herein are done using the default parameters provided with these algorithms. Examples of such sequences are allelic or species variants of the specific OSCAR genes and gene products of the invention including, for example, allelic or species variants of the OSCAR polypeptide sequences depicted in FIGS. 1C, 3B, 4B, 5B, 24B, 25B, 26B and 27B (SEQ ID NOS:3, 7, 9, 11, 25, 27, 29 and 31 respectively). Sequences that are substantially homologous can be identified by comparing the sequences using standard software available in sequence data banks.

In other embodiments, variants of an OSCAR polypeptide (including analogs and homologs) are polypeptides encoded by nucleic acid molecules that hybridize to the complement of a nucleic acid molecule encoding an OSCAR polypeptide; e.g., in a Southern hybridization experiment under defined conditions. For example, in a particular embodiment analogs and/or homologs of an OSCAR polypeptide comprise amino acid sequence encoded by nucleic acid molecules that hybridize to a complement of an OSCAR nucleic acid sequence, such as the any of the coding sequences set forth in FIGS. 1A, 1B 2A, 26A and 27A (SEQ ID NOS:1, 2, 4, 30, and 31respectively) and in FIGS. 3A, 4A 5A, 24A and 25A (SEQ ID NOS:6, 8, 10, 26 and 28 respectively) under highly stringent hybridization conditions that comprise 50% formamnide and 5× or 6×SSC. In other embodiments, the analogs and/or homologs of an OSCAR polypeptide may comprise amino acid sequences encoded by nucleic acid molecules that hybridize to a complement of an OSCAR nucleic acid sequence (e.g., the coding sequence set forth in FIGS. 1A, 1B, 2A, 3A, 4A, 5A, 24A, 25A, 26A and 27A and in SEQ ID NOS:1-2, 4, 6, 8, 10, 26, 28, 30 and 31 respectively) under moderately stringent hybridization conditions (i e., 40% formamide with 5× or 6×SSC), or under low stringency conditions (e.g., in 5×SSC, 0.1% SDS, 0.25% milk, no formamide, 30% formamide, 5×SSC, or 0.5% SDS).

In still other embodiments, variants, including analogs homalogs and orthologs, of an OSCAR polypeptide can also be identified by isolating variant OSCAR genes, e.g., by PCR using degenerate oligonucleotide primers designed on the basis of amino acid sequence of the OSCAR polypeptide and as described below.

Derivatives of the OSCAR polypeptides of the invention further include, but are by no means limited to, phosphorylated OSCAR, myristylated OSCAR, methylated OSCAR and other OSCAR polypeptides that are chemically modified. OSCAR polypeptide of the invention also include labeled variants; for example, radio-labeled with iodine or phosphorous (see, e.g., EP 372707B) or other detectable molecule such as, but by no means limited to, biotin, a fluorescent dye (e.g, Cy5 or Cy3), a chelating group complexed with a metal ion, a chromophore or fluorophore, a gold colloid, a particle such as a latex bead, or attached to a water soluble polymer.

Chemical modification of a biologically active component or components of OSCAR nucleic acids or polypeptides may provide additional advantages under certain circumstances. See, for example, U.S. Pat. No. 4,179,337 issued Dec. 18, 1970 to Davis et al. Also, for a review see Abuchowski et al., in Enzymes as Drugs (J. S. Holcerberg and J. Roberts, eds. 1981), pp.367-383. A review article describing protein modification and fusion proteins is found in Francis, Focus on Growth Factors 1992, 3:4-10, Mediscript: Mountview Court, Friern Barnet Lane, London N20, OLD, UK.

### OSCAR Nucleic Acids

OSCAR nucleic acid molecules of the invention are also defined above, and include DNA and RNA molecules as well as nucleic acid molecules comprising any of the modification (e.g., modified bases and/or backbone) described above. In general, an OSCAR nucleic acid molecule comprises a nucleic acid sequence that encodes an OSCAR polypeptide, the complement of a nucleic acid sequence that encodes an OSCAR polypeptide, and fragments thereof. Thus, in one preferred embodiment, the OSCAR nucleic acid molecules of the invention comprise nucleotide sequences that encode the amino acid sequence set forth in FIG. 1C (SEQ ID NO:3), such as the particular OSCAR nucleic acid sequences set forth in FIGS. 1A and 1B (SEQ ID NOS:1 and 2, respectively). In another preferred embodiment, the nucleic acid molecules of the invention comprise nucleotide sequences that encode the amino acid sequence set forth in FIG. 26B (SEQ ID NO:29), such as the particular OSCAR nucleic acid sequences set forth in FIG. 26A (SEQ ID NO.30). In yet another preferred embodiment, the nucleic acid molecules of the invention comprise nucleotide sequences that encode the amino acid sequence set forth in FIG. 27B (SEQ ID NO:31), such as the particular OSCAR nucleic acid sequences set forth in FIG. 27A (SEQ ID NO:32).

In another preferred embodiment, OSCAR nucleic acid molecules of the invention comprise nucleotide sequences that encode the amino acid sequence set forth in FIG. 3B (SEQ ID NO:7) for the C18 human OSCAR isoform described supra, including the particular OSCAR nucleic acid sequence set forth in FIG. 3A (SEQ ID NO:6). Preferably, nucleic acid 328 of that exemplary OSCAR sequence (i.e., the exemplary sequence shown in FIG. 3A and in SEQ ID NO:6) is a guanine. However, in an exemplary alternative embodiment nucleic acid 328 can be a thymine.

In still another preferred embodiment, the OSCAR nucleic acid molecules of the invention comprise nucleotide sequences that encode the amino acid sequence set forth in FIG. 4B (SEQ ID NO:9) for the C16 human OSCAR isoform described supra, including the particular OSCAR nucleic acid sequence set forth in FIG. 4A (SEQ ID NO:8). In yet another preferred embodiment, the OSCAR nucleic acid molecules of the invention comprises nucleotide sequences that encode the amino acid sequence set forth in FIG. 5B (SEQ ID NO:11) for the C10 human OSCAR isoform described supra, including the particular OSCAR nucleic acid sequence set forth in FIG. 5A (SEQ ID NO:10).

In another preferred embodiment, the OSCAR nucleic acid molecules of the invention comprise nucleotide sequences that encode the amino acid sequence set forth in FIG. 24B (SEQ ID NO:25) for the S1 human OSCAR isoform, including the particular OSCAR nucleic acid sequence set forth in FIG. 24A. In yet another preferred embodiment, the OSCAR nucleic acid molecules of the invention comprise nucleotide sequences that encode the amino acid sequence set forth in FIG. 25B (SEQ ID NO:27) for the S2 human OSCAR isoform, including the particular OSCAR nucleic acid sequence set forth in FIG. 25A (SEQ ID NO: 28).

In still other embodiments, the OSCAR nucleic acid molecules of the invention comprise nucleic acid sequences that encode one or more domains of an OSCAR polypeptide (e.g., a signal sequence domain, one or more Ig-like domains, a transmembrane domain or a cytoplasmic tail domain), or nucleic acid sequences that encode any combination of domains of an OSCAR polypeptide.

The OSCAR nucleic acid molecules of the present invention also comprise genomic OSCAR nucleotide sequences for an OSCAR gene. For example, FIGS. 7A-D (SEQ ID NO: 12) set forth the sequences from a region of human chromosome 19 which comprises the nucleotide sequence of a human OSCAR gene. Nucleic acid molecules comprising these nucleotide sequences are therefore among the OSCAR nucleic acids of the present invention. For example, in one embodiment, the OSCAR nucleic acid molecules of the invention comprise nucleotide sequences from one or more of the intron or exon sequences described in TABLE 1, supra and illustrated in FIGS. 7A-D. In other embodiments, the OSCAR nucleic acid molecules of the invention comprise nucleotide sequences for a combination of exons and/or introns of an OSCAR gene.

The OSCAR nucleic acid molecules of the present invention may also comprise nucleic acid sequences that encode fragments (e.g., epitopes) of an OSCAR polypeptide. Such fragments include, for example, polynucleotides encoding the nucleic acid sequence set forth in FIG. 2A (SEQ ID NO:4), as well as other nucleic acid sequences that encode the polypeptide sequence set forth in FIG. 2B (SEQ ID NO:5).

The OSCAR nucleic acid molecules of the invention also include nucleic acid molecules that comprise coding sequences for modified OSCAR polypeptides (e.g., having amino acid substitutions, deletions or truncations) and for variants (including analogs and homologs from the same and different species) of OSCAR polypeptides. In preferred embodiments, such nucleic acid molecules have at least 50%, preferably at least 75% and more preferably at least 90% sequence identity to an OSCAR coding nucleotide sequence such as the coding sequences set forth in FIGS. 1A-B, 3A, 4A, 5A, 7A-D, 24A, 25A, 26A and 27A (SEQ ID NOS: 1-2, 6, 8, 10, 12, 26, 28, 30 and 32 respectively). Alternatively, nucleic acid molecules of the invention may also be ones that hybridize to an OSCAR nucleic acid molecule, e.g., in a Southern blot assay under defined conditions. For example, in a specific embodiment a labeled OSCAR cDNA hybridizes to one or more human genomic fragments, including a 1.65 kb EcoRI fragment and a 5.5 kb Bgl II fragment.

In a particular embodiment an OSCAR nucleic acid molecule of the invention comprises a nucleotide sequence which hybridizes to a complement of an OSCAR nucleic acid sequence, such as the any of the coding sequences set forth in FIGS. 1A-B, 3A, 4A,5A, 7A-D, 24A, 25A, 26A and 27A (SEQ ID NOS:1-2, 6, 8, 10, 12, 26, 28, 30 and 32 respectively) under highly stringent hybridization conditions that comprise 50% fornamide and 5× or 6×SSC. In other embodiments, the nucleic acid molecules hybridize to a complement of an OSCAR nucleic acid sequence (e.g., the coding sequence set forth in FIGS. 1A-B, 3A, 4A, 5A, 7A-D, 24A, 25A, 26A and 27A) under moderately stringent hybridization conditions (i.e., 40% formamide with 5× or 6×SSC), or under low stringency conditions (e.g., in 5×SSC, 0.1% SDS, 0.25% milk, no formamide, 30% formamide, 5×SSC, or 0.5% SDS). Particularly preferred hybridization conditions comprise hybridization at 42° C. in a low stringency hybridization buffer (e.g., 30% formamide, 10 mM Tris pH 7.6, 2.5× Denhardt's solution, 5×SSC, 0.5% SDS and 1.5 mg/ml sonicated salmon sperm DNA) followed by washing (preferably twice) at 50° C. using a low stringency washing buffer (e.g., 0.5×SSC and 1% SDS). For example, the Examples, infra, describe experiments in which fragments of mouse and human genomic DNA were hybridized to an OSCAR nucleic acid sequence derived from the OSCAR clone OSL178 (SEQ ID NO:4). Such genomic sequences are therefore part of the OSCAR nucleic acid sequences of the present invention.

Alternatively, a nucleic acid molecule of the invention may hybridize, under the same defined hybridization conditions, to the complement of a fragment of a nucleotide sequence encoding a full length OSCAR polypeptide, such as the fragment set forth in FIG. 2A (SEQ ID NO:4) or to another nucleic acid molecule that encodes the OSCAR polypeptide fragment depicted in FIG. 2B (SEQ ID NO:5). For instance, the Examples, infra, describe the identification of OSCAR mRNA molecules of 4.0 kb, 1.8 kb and 1.1 kb apparent length that hybridize to the OSCAR nucleic acid fragment contained in the clone OCL178 and set forth in FIG. 2A (SEQ ID NO:4). The Examples also describe the identification of both murine an human genomic DNA fragments that hybridize to the OCL178 nucleic acid. Such nucleic acids are therefore exemplary embodiments of OSCAR nucleic acid molecules of the present invention.

In other embodiments, the nucleic acid molecules of the invention comprise fragments of a full length OSCAR sequence. For example, in preferred embodiments such OSCAR nucleic acid fragments comprise a nucleotide sequence that corresponds to a sequence of at least 10 nucleotides, preferably at least 15 nucleotides and more preferably at least 20 nucleotides of a full length coding OSCAR nucleotide sequence. In specific embodiments, the fragments correspond to a portion (e.g., of at least 10, 15 or 20 nucleotides) of the OSCAR coding sequences set forth in FIGS. 1A-B, 3A, 4A, 5A, 7A-D, 24A, 25A, 26A and 27A (SEQ ID NOS:1-2, 6, 8, 10, 12, 26,28, 30 and 32 respectively) or of other nucleotide sequences encoding the polypeptide sequences set forth in FIGS. 1C, 2B, 3B, 4B, 5B, 24B, 25B, 26B and 27B (SEQ ID NOS:3, 5, 7, 9 11, 25, 27, 29 and 31 respectively).

In other preferred embodiments the OSCAR nucleic acid fragments comprise sequences of at least 10, preferably at least 15, and more preferably at least 20 nucleotides that are complementary and/or hybridize to a full length coding OSCAR nucleic acid sequence (e.g., in the sequences set forth in FIGS. 1A-B, 3A, 4A, 5A, 7A-D, 24A, 25A, 26A and 27A (SEQ ID NOS:1-2, 6, 8, 10, 12, 26, 28, 30 and 32 respectively), or a fragment thereof (e.g., in the sequence set forth in FIG. 2A and in SEQ ID NO:4). Suitable hybridization conditions for such oligonucleotides are described supra, and include washing in 6×SSC/0.05% sodium pyrophosphate. Because the melting temperature of oligonucleotides will depend on the length of the oligonucleotide sequence, suitable hybridization temperatures will vary depending upon the oligoncucleotide molecules used. Exemplary temperatures will be 37° C. (for 14-base oligonucleotides), 48° C. (for 17-base oligoncucleotides), 55° C. (for 20-base oligonucleotides) and 60° C. (for 23-base oligonucleotides).

The nucleic acid molecules of the invention also include “chimeric” OSCAR nucleic acid molecules. Such chimeric nucleic acid molecules are polynucleotides which comprise at least one OSCAR nucleic acid sequence (which may be any of the full length or partial OSCAR nucleic acid sequences described above), and also at least one non-OSCAR nucleic acid sequence. For example, the non-OSCAR nucleic acid sequence may be a regulatory sequence (for example a promoter sequence) that is derived from another, non-OSCAR gene and is not normally associated with a naturally occurring OSCAR gene. The non-OSCAR nucleic acid sequence may also be a coding sequence for another, non-OSCAR polypeptide, such as FLAG, a histidine tag, glutathione S-transferase (GST), hemaglutinin, β-galactosidase, thioreductase or an immunoglobulin domain or domains (for example, an Fc region). In preferred embodiments, a chimeric nucleic acid molecule of the invention encodes an OSCAR fusion polypeptide of the invention.

Nucleic acid molecules comprising such fragments are useful, for example, as oligonucleotide probes and primers (e.g., PCR primers) to detect and amplify other nucleic acid molecules encoding an OSCAR polypeptide, including genes that encode variant OSCAR polypeptides such as OSCAR analogs and homologs. Oligonucleotide fragments of the invention may also be used, e.g., as antisense nucleic acids, triple helix forming oligonucleotides or as ribozymes; e.g., to modulate levels of OSCAR gene expression or transcription in cells.

OSCAR nucleic acid molecules of the invention, whether genomic DNA, cDNA or otherwise, can be isolated from any source, including, for example, murine and human cDNA or genomic libraries. Methods for obtaining OSCAR genes are well known in the art, as described above (see, e.g., Sambrook et al., 1989, supra).

The DNA may be obtained by standard procedures known in the art from cloned DNA (for example, from a DNA “library”), and preferably is obtained from a cDNA library prepared from tissues with high level expression of the protein (e.g., an osteoclast library, since these cells evidence highest levels of expression of OSCAR). In one preferred embodiment, the DNA is obtained from a “subtraction” library, as described in the Examples, infra, to enrich the library for cDNAs of genes specifically expressed by a particular cell type. For example, as described in the Examples, infra, a osteoclast-macrophage subtraction library may be constructed in which a substantial fraction of cDNAs derived from osteoclast that are also expressed by macrophages are removed. Use of such a subtraction library may increase the likelihood of isolating cDNA for a gene, such as OSCAR, that is specifically expressed by osteoclast and not by macrophages. In other embodiments, a library may be prepared by chemical synthesis, by cDNA cloning, or by the cloning of genomic DNA or fragments thereof, purified from the desired cell (See, for example, Sambrook et al., 1989, supra; Glover, D. M. ed., 1985, DNA Cloning: A Practical Approach, MRL Press, Ltd., Oxford, U.K. Vols. I and II).

Clones derived from genomic DNA may contain regulatory and intron DNA region in addition to coding regions. Clones derived from cDNA generally will not contain intron sequences. Whatever the source, the gene should be molecularly cloned into a suitable vector for propagation of the gene. Identification of the specific DNA fragment containing the desired OSCAR gene may be accomplished in a number of ways. For example, a portion of an OSCAR gene exemplified infra can be purified and labeled to prepare a labeled probe (Benton & Davis, Science 1977, 196:180; Grunstein & Hogness, Proc. Natl. Acad. Sci. U.S.A. 1975, 72:3961). Those DNA fragments with substantial homology to the probe, such as an allelic variant from another individual, will hybridize. In a specific embodiment, highest stringency hybridization conditions are used to identify a homologous OSCAR gene.

Further selection can be carried out on the basis of the properties of the gene, e.g., if the gene encodes a protein product having the isoelectric, electrophoretic, amino acid composition, partial or complete amino acid sequence, antibody binding activity, or ligand binding profile of OSCAR protein as disclosed herein. Thus, the presence of the gene may be detected by assays based on the physical, chemical, immunological, or functional properties of its expressed product.

Other DNA sequences which encode substantially the same amino acid sequence as an OSCAR gene may be used in the practice of the present invention. These include but are not limited to allelic variants, species variants, sequence conservative variants, and functional variants. In particular, the nucleic acid sequences of the invention include both “function-conservative variants” and “sequence-conservative variants”. Function-conservative variants of a nucleic acid are those nucleic acids which encode a function-conservative variant of a polypeptide, as defined supra. “Sequence-conservative variants” of a nucleic acid are ones that have a different polynucleotide sequence but encode the same amino acid sequence.

Amino acid substitutions may also be introduced to substitute an amino acid with a particularly preferable property. For example, a Cys may be introduced a potential site for disulfide bridges with another Cys.

The genes encoding OSCAR derivatives and analogs of the invention can be produced by various methods known in the art. The manipulations which result in their production can occur at the gene or protein level. For example, the cloned OSCAR gene sequence can be modified by any of numerous strategies known in the art (Sambrook et al., 1989, supra). The sequence can be cleaved at appropriate sites with restriction endonuclease(s), followed by further enzymatic modification if desired, isolated, and ligated in vitro. In the production of the gene encoding a derivative or analog of OSCAR, care should be taken to ensure that the modified gene remains within the same translational reading frame as the OSCAR gene, uninterrupted by translational stop signals, in the gene region where the desired activity is encoded.

Additionally, the OSCAR-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further iii vitro modification.. Modifications can also be made to introduce restriction sites and facilitate cloning the OSCAR gene into an expression vector. Any technique for mutagenesis known in the art can be used, including but not limited to, ill vitro site-directed mutagenesis (Hutchinson, C., et al., J. Biol. Chem. 253:6551, 1978; Zoller and Smith, DNA 3:479-488, 1984; Oliphant et al., Gene 44:177, 1986; Hutchinson et al., Proc. Natl. Acad. Sci. U.S.A. 83:710, 1986), use of TAB” linkers (Pharmacia), etc. PCR techniques are preferred for site directed mutagenesis (see Higuchi, 1989, “Using PCR to Engineer DNA”, in PCR Technology: Principles and Applications for DNA Amplification, H. Erlich, ed., Stockton Press, Chapter 6, pp. 61-70).

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, pKK plasmids (Clonetech), pET plasmids (Novagen, Inc., Madison, Wis.), pRSET or pREP plasmids (Invitrogen, San Diego, Calif.), or pMAL plasmids (New England Biolabs, Beverly, Mass.), etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences.

Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences form the yeast 2m plasmid.

### Expression of OSCAR Polypeptides

The nucleotide sequence coding for OSCAR, or antigenic fragment, derivative or analog thereof, or a functionally active derivative, including a chimeric protein, thereof, can be inserted into an appropriate expression vector, i.e., a vector which contains the necessary elements for the transcription and translation of the inserted protein-coding sequence. Thus, a nucleic acid encoding OSCAR of the invention can be operationally associated with a promoter in an expression vector of the invention. Both cDNA and genomic sequences can be cloned and expressed under control of such regulatory sequences. Such vectors can be used to express functional or functionally inactivated OSCAR polypeptides.

The necessary transcriptional and translational signals can be provided on a recombinant expression vector.

Potential host-vector systems include but are not limited to mammalian cell systems transfected with expression plasmids or infected with virus (e.g., vaccinia virus, adenovirus, adeno-associated virus, herpes virus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Expression of OSCAR protein may be controlled by any promoter/enhancer element known in the art, but these regulatory elements must be functional in the host selected for expression. Promoters which may be used to control OSCAR gene expression include, but are not limited to, cytomegalovirus (CMV) promoter (U.S. Pat. Nos. 5,385,839 and No. 5,168,062), the SV40 early promoter region (Benoist and Chambon, 1981, Nature 290:304-310), the promoter contained in the 3′ long terminal repeat of Rous sarcoma virus (Yamamoto, et al., Cell 22:787-797, 1980), the herpes thymidine kinase promoter (Wagner et al., Proc. Natl. Acad. Sci. U.S.A. 78:1441-1445, 1981), the regulatory sequences of the metallothionein gene (Brinster et al., Nature 296:3942, 1982); prokaryotic expression vectors such as the b-lactamase promoter (Villa-Komaroff, et al., Proc. Natl. Acad. Sci. U.S.A. 75:3727-3731, 1978), or the tac promoter (DeBoer, et al., Proc. Natl. Acad. Sci. U.S.A. 80:21-25, 1983); see also “Useful proteins from recombinant bacteria” in Scientific American, 242:74-94, 1980; promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature 315:338-340, 1985; Kollias et al., Cell 46:89-94, 1986), hematopoietic stem cell differentiation factor promoters, erythropoietin receptor promoter (Maouche et al., Blood, 15:2557, 1991), etc.

Indeed, any type of plasmid, cosmid, YAC or viral vector may be used to prepare a recombinant nucleic acid construct which can be introduced to a cell, or to tissue, where expression of an OSCAR gene product is desired. Alternatively, wherein expression of a recombinant OSCAR gene product in a particular type of cell or tissue is desired, viral vectors that selectively infect the desired cell type or tissue type can be used.

In another embodiment, the invention provides methods for expressing OSCAR polypeptides by using a non-endogenous promoter to control expression of an endogenous OSCAR gene within a cell. An endogenous OSCAR gene within a cell is an OSCAR gene of the present invention which is ordinarily (i.e., naturally) found in the genome of tht cell. A non-endogenous promoter, however, is a promoter or other nucleotide sequence that may be used to control expression of a gene but is not ordinarily or naturally associated with the endogenous OSCAR gene. As an example, methods of homologous recombination may be employed (preferably using non-protein encoding OSCAR nucleic acid sequences of the invention) to insert an amplifiable gene or other regulatory sequence in the proximity of an endogenous OSCAR gene. The inserted sequence may then be used, e.g., to provide for higher levels of OSCAR gene expression than normally occurs in that cell, or to overcome one or more mutations in the endogenous OSCAR regulatory sequences which prevent normal levels of OSCAR gene expression (for example, in osteoclast cells). Such methods of homologous recombination are well known in the art. See, for example, International Patent Publication No. WO 91/06666, published May 16, 1991 by Skoultchi; International Patent Publication No. WO 91/099555, published Jul. 11, 1991 by Chappel; and International Patent Publication No. WO 90/14092, published Nov. 29, 1990 by Kucherlapati and Campbell.

Soluble forms of the protein can be obtained by collecting culture fluid, or solubilizing inclusion bodies, e.g., by treatment with detergent, and if desired sonication or other mechanical processes, as described above. The solubilized or soluble protein can be isolated using various techniques, such as polyacrylamide gel electrophoresis (PAGE), isoelectric focusing, 2-dimensional gel electrophoresis, chromatography (e.g., ion exchange, affinity, immunoaffinity, and sizing column chromatography), centrifugation, differential solubility, immunoprecipitation, or by any other standard technique for the purification of proteins.

### Expression Vectors

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMal-C2, pET, pGEX (Smith et al., Gene 67:31-40, 1988), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Preferred vectors are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant OSCAR protein or polypeptide domain fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published October 1995.

According to the present invention, vectors may be specifically targeted to osteoclast cells using, for example, an OSCAR-specific antibody (i.e. an antibody that specifically binds to an OSCAR gene product) or using an OSCAR binding partner such as an OSCAR-specific ligand. Vectors may also be specifically targeted to osteoclast cells using fragments (e.g., peptide or polypeptide fragments) of an OSCAR binding partner, particularly fragments which comprise an OSCAR binding sequence. Such methods may be used to target vectors expressing any gene to osteoclast cells, including but not limited to vectors that express OSCAR specific antisense nucleic acids or OSCAR specific ribozymes.

Similarly, the invention also permits specific targeting of osteoblast cells and embryonic fibroblast cells, as well as other cells (such as NIH 3T3, ST2, Mlg, UMR106, HEK293, HEK293T, hFOB1.19, and COS-1 cells) that express an OSCAR-specific ligand or an OSCAR binding partner on the cell surface, by using an OSCAR polypeptide as the targeting entity.

Viral vectors commonly used for in vivo or ex vivo targeting and therapy procedures are DNA-based vectors and retroviral vectors. Methods for constructing and using viral vectors are known in the art (see, e.g., Miller and Rosman, BioTechniques, 7:980-990, 1992). Preferably, the viral vectors are replication defective, that is, they are unable to replicate autonomously in the target cell. In general, the genome of the replication defective viral vectors which are used within the scope of the present invention lack at least one region which is necessary for the replication of the virus in the infected cell. These regions can either be eliminated (in whole or in part), be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome which are necessary for encapsidating the viral particles.

DNA viral vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Molec. Cell. Neurosci. 2:320-330, 1991), defective herpes virus vector lacking a glyco-protein L gene (Patent Publication RD 371005 A), or other defective herpes virus vectors (International Patent Publication No. WO 94/21807, published Sep. 29, 1994; International Patent Publication No. WO 92/05263, published Apr. 2, 1994); an attenuated adenovirus vector, such as the vector described by Stratford-Perricaudet et al. (J. Clin. Invest. 90:626-630, 1992; see also La Salle et al, Science 259:988-990, 1993); and a defective adeno-associated virus vector (Samulski et al., J. Virol. 61:3096-3101, 1987; Samulski et al., J. Virol. 63:3822-3828, 1989; Lebkowski et al., Mol. Cell. Biol. 8:3988-3996, 1988).

Various companies produce viral vectors commercially, including but by no means limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

In another embodiment, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner, et. al., Proc. Natl. Acad. Sci. U.S.A. 84:7413-7417, 1987; Felgner and Ringold, Science 337:387-388,1989; see Mackey, et al., Proc. Natl. Acad. Sci. U.S.A. 85:8027-8031, 1988; Ulmer et al., Science 259:1745-1748, 1993). Useful lipid compounds and compositions for transfer of nucleic acids are described in International Patent Publications WO95/18863 and WO96/17823, and in U.S. Pat. No. 5,459,127. Lipids may be chemically coupled to other molecules for the purpose of targeting (see Mackey, et. al., supra). Targeted peptides, e.g., hormones or neurotransmitters, and proteins such as antibodies, or non-peptide molecules could be coupled to liposomes chemically. Other molecules are also useful for facilitating transfection of a nucleic acid in vivo, such as a cationic oligopeptide (e.g., International Patent Publication WO95/21931), peptides derived from DNA binding proteins (e.g., International Patent Publication WO96/25508), or a cationic polymer (e.g., International Patent Publication WO95/21931).

It is also possible to introduce the vector in vivo as a naked DNA plasmid. Naked DNA vectors for gene therapy can be introduced into the desired host cells by methods known in the at, e.g., electroporation, microinjection, cell fusion, DEAE dextran, calcium phosphate precipitation, use of a gene gun, or use of a DNA vector transporter (see, e.g., Wu et al., J. Biol. Chem. 267:963-967, 1992; Wu and Wu, J. Biol. Chem. 263:1462 1-14624, 1988; Hartmut et al., Canadian Patent Application No. 2,012,311, filed Mar. 15, 1990; Williams et al., Proc. Natl. Acad. Sci. USA 88:2726-2730, 1991). Receptor-mediated DNA delivery approaches can also be used (Curiel et al., Hum. Gene Ther. 3:147-154, 1992; Wu and Wu, J. Biol. Chem. 262:4429-4432, 1987). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C.P. Acad. Sci., 321:893, 1998; WO 99/01157; WO 99/01158; WO 99/01175).

Preferably, for in vivo administration, an appropriate immunosuppressive treatment is employed in conjunction with the viral vector, e.g., adenovinis vector, to avoid immuno-deactivation of the viral vector and transfected cells. For example, immunosuppressive cytokines, such as interleukin-12 (IL-12), interferon-g (IFN-γ), or anti-CD4 antibody, can be administered to block humoral or cellular immune responses to the viral vectors (see, e.g., Wilson, Nature Medicine, 1995). In that regard, it is advantageous to employ a viral vector that is engineered to express a minimal number of antigens.

### Antibodies to OSCAR

Antibodies to OSCAR are useful, inter alia, for diagnostics and intracellular regulation of OSCAR activity, as set forth below. According to the invention, OSCAR polypeptides produced recombinantly or by chemical synthesis, and fragments or other derivatives or analogs thereof, including fusion proteins, may be used as an immunogen to generate antibodies that recognize the OSCAR polypeptide. Such antibodies include but are not limited to polyclonal, monoclonal, chimeric, single chain, Fab fragments, and an Fab expression library. Such an antibody is preferably specific for (i.e., specifically binds to) a human OSCAR or a murine OSCAR. However, the antibody may, alternatively, be specific for an OSCAR ortholog from some other species of organism, preferably a mammalian species. The antibody may recognize a mutant form of OSCAR, or wild-type OSCAR, or both.

Various procedures known in the art may be used for the production of polyclonal antibodies to OSCAR polypeptide or derivative or analog thereof. For the production of antibody, various host animals can be immunized by injection with the OSCAR polypeptide, or a derivative (e.g., fragment or fusion protein) thereof, including but not limited to rabbits, mice, rats, sheep, goats, etc. In one embodiment, the OSCAR polypeptide or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the OSCAR polypeptide, or fragment, analog, or derivative thereof, any technique that provides for the production of antibody molecules by continuous cell lines in culture may be used. These include but are not limited to the hybridoma technique originally developed by Kohler and Milstein (Nature 1975, 256:495-497), as well as the trioma technique, the human B-cell hybridoma technique (Kozbor et al., Immunology Today 1983, 4:72; Cote et al., Proc. Natl. Acad. Sci. U.S.A. 1983, 80:2026-2030), and the EBV-hybridoma technique to produce human monoclonal antibodies (Cole et al., in Monoclonal Antibodies and Cancer Therapy, Alan R. Liss, Inc., 1985, pp. 77-96). In an additional embodiment of the invention, monoclonal antibodies can be produced in germ-free animals (International Patent Publication No. WO 89/12690). In fact, according to the invention, techniques developed for the production of “chimeric antibodies” (Morrison et al., J. Bacteriol. 1984, 159:870; Neuberger et al., Nature 1984, 312:604-608; Takeda et al., Nature 1985, 314:452-454) by splicing the genes from a mouse antibody molecule specific for an OSCAR polypeptide together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

According to the invention, techniques described for the production of single chain antibodies (U.S. Pat. Nos. 5,476,786, 5,132,405, and 4,946,778) can be adapted to produce OSCAR polypeptide-specific single chain antibodies. An additional embodiment of the invention utilizes the techniques described for the construction of Fab expression libraries (Huse et al., Science 1989, 246:1275-1281) to allow rapid and easy identification of monoclonal Fab fragments with the desired specificity for an OSCAR polypeptide, or its derivatives, or analogs.

In the production and use of antibodies, screening for or testing with the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g, gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of an OSCAR polypeptide, one may assay generated hybridomas for a product which binds to an OSCAR polypeptide fragment containing such epitope. For selection of an antibody specific to an OSCAR polypeptide from a particular species of animal, one can select on the basis of positive binding with OSCAR polypeptide expressed by or isolated from cells of that species of animal.

The foregoing antibodies can be used in methods known in the art relating to the localization and activity of the OSCAR polypeptide, e.g., for Western blotting, imaging OSCAR polypeptide in situ, measuring levels thereof in appropriate physiological samples, etc. using any of the detection techniques mentioned above or known in the art.

Such antibodies can also be used in assays for ligand binding, e.g., as described in U.S. Pat. No. 5,679,582. Antibody binding generally occurs most readily under physiological conditions, e.g., pH of between about 7 and 8, and physiological ionic strength. The presence of a carrier protein in the buffer solutions stabilizes the assays. While there is some tolerance of perturbation of optimal conditions, e.g., increasing or decreasing ionic strength, temperature, or pH, or adding detergents or chaotropic salts, such perturbations will decrease binding stability.

In still other embodiments, anti-OSCAR antibodies may also be used to isolate cells which express an OSCAR polypeptide, e.g., osteoclast cells, by panning or related immunoadsorption techniques.

In a specific embodiment, antibodies that agonize or antagonize the activity of OSCAR polypeptide can be generated. In particular, intracellular single chain Fv antibodies can be used to regulate (inhibit) OSCAR activity (Marasco et al., Proc. Natl. Acad. Sci. U.S.A. 1993, 90:7889-7893; Chen., Mol. Med. Today 1997, 3:160-167; Spitz et al., Anticancer Res. 1996, 16:3415-22; Indolfi et al., Nat. Med. 1996, 2:634-635; Kijma et al., Pharmacol. Ther. 1995, 68:247-267). Such antibodies can be tested using the assays described infra for identifying ligands.

Antibodies can also be used to create immunotoxins, as discussed in the section on screening assays, infra.

### In Vivo Testing Using Transgenic Animals

Transgenic mammals can be prepared for evaluating the molecular mechanisms of OSCAR, and particularly human OSCAR-induced signaling. Such mammals provide excellent models for screening or testing drug candidates. Thus, human OSCAR “knock-in” mammals can be prepared for evaluating the molecular biology of this system in greater detail than is possible with human subjects. It is also possible to evaluate compounds or diseases on “knockout” animals, e.g., to identify a compound that can compensate for a defect in OSCAR activity. Both technologies permit manipulation of single units of genetic information in their natural position in a cell genome and to examine the results of that manipulation in the background of a terminally differentiated organism. Trangenic mammals can be prepared by any method, including but not limited to modification of embryonic stem (ES) cells and heteronuclear injecion into blast cells.

A “knock-in” mammal is a mammal in which an endogenous gene is substituted with a heterologous gene (Roamer et al., New Biol. 1991, 3:331). Preferably, the heterologous gene is “knocked-in” to a locus of interest, either the subject of evaluation (in which case the gene may be a reporter gene; see Elegant et al., Proc. Natl. Acad. Sci. USA 1998, 95:11897) of expression or function of a homologous gene, thereby linking the heterologous gene expression to transcription from the appropriate promoter. This can be achieved by homologous recombination, transposon (Westphal and Leder, Curr Biol 1997, 7:530), using mutant recombination sites (Araki et al., Nucleic Acids Res 1997, 25:868) or PCR (Zhang and Henderson, Biotechniques 1998, 25:784).

A “knockout mammal” is an mammal (e.g., mouse) that contains within its genome a specific gene that has been inactivated by the method of gene targeting (see, e.g., U.S. Pat. Nos. 5,777,195 and 5,616,491). A knockout mammal includes both a heterozygote knockout (i.e., one defective allele and one wild-type allele) and a homozygous mutant. Preparation of a knockout mammal requires first introducing a nucleic acid construct that will be used to suppress expression of a particular gene into an undifferentiated cell type termed an embryonic stem cell. This cell is then injected into a mammalian embryo. A mammalian embryo with an integrated cell is then implanted into a foster mother for the duration of gestation. Zhou, et al. (Genes and Development, 1995, 9:2623-34) describes PPCA knock-out mice.

The term “knockout” refers to partial or complete suppression of the expression of at least a portion of a protein encoded by an endogenous DNA sequence in a cell. The term “knockout construct” refers to a nucleic acid sequence that is designed to decrease or suppress expression of a protein encoded by endogenous DNA sequences in a cell. The nucleic acid sequence used as the knockout construct is typically comprised of (1) DNA from some portion of the gene (exon sequence, intron sequence, and/or promoter sequence) to be suppressed and (2) a marker sequence used to detect the presence of the knockout construct in the cell. The knockout construct is inserted into a cell, and integrates with the genomic DNA of the cell in such a position so as to prevent or interrupt transcription of the native DNA sequence. Such insertion usually occurs by homologous recombination (i.e., regions of the knockout construct that are homologous to endogenous DNA sequences hybridize to each other when the knockout construct is inserted into the cell and recombine so that the knockout construct is incorporated into the corresponding position of the endogenous DNA). The knockout construct nucleic acid sequence may comprise 1) a full or partial sequence of one or more exons and/or introns of the gene to be suppressed, 2) a full or partial promoter sequence of the gene to be suppressed, or 3) combinations thereof. Typically, the knockout construct is inserted into an embryonic stem cell (ES cell) and is integrated into the ES cell genomic DNA, usually by the process of homologous recombination. This ES cell is then injected into, and integrates with, the developing embryo.

The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, many progeny of the cell will no longer express the gene at least in some cells, or will express it at a decreased level, as the DNA is now disrupted by the antibiotic resistance gene.

Generally, for homologous recombination, the DNA will be at least about 1 kilobase (kb) in length and preferably 34 kb in length, thereby providing sufficient complementary sequence for recombination when the knockout construct is introduced into the genomic DNA of the ES cell (discussed below).

Included within the scope of this invention is a mammal in which two or more genes have been knocked out or knocked in, or both. Such mammals can be generated by repeating the procedures set forth herein for generating each knockout construct, or by breeding to mammals, each with a single gene knocked out, to each other, and screening for those with the double knockout genotype.

Regulated knockout animals can be prepared using various systems, such as the tet-repressor system (see U.S. Pat. No. 5,654,168) or the Cre-Lox system (see U.S. Pat. No. 4,959,317 and U.S. Pat. No. 5,801,030).

In another series of embodiments, transgenic animals are created in which (i) a human OSCAR is stably inserted into the genome of the transgenic animal; and/or (ii) the endogenous OSCAR genes are inactivated and replaced with their human counterparts (see, e.g., Coffman, Semin. Nephrol. 1997, 17:404; Esther et al., Lab. Invest. 1996, 74:953; Murakami et al., Blood Press. Suppl. 1996, 2:36). Such animals can be treated with candidate compounds and monitored for neuronal development, neurodegeneration, or efficacy of a candidate therapeutic compound.

### Applications and Uses

Described herein are various applications and uses for OSCAR gene sequences (including fragments of full length OSCAR gene sequences), OSCAR polypeptides (including fragments of full length OSCAR proteins and OSCAR fusion polypeptides) and of antibodies directed against OSCAR nucleic acids and OSCAR polypeptides (including fragments of full length OSCAR genes and proteins). Such applications may include, for example, both prognostic and diagnostic applications for evaluating bone growth related disorders associated with an OSCAR gene, and OSCAR gene product or an OSCAR polypeptide, including the identification of subjects having such a disorder or having a predisposition to such a disorder. Additionally, such applications may include methods for treating disorders associated with an OSCAR gene, with an OSCAR gene product or with an OSCAR polypeptide, as well as screening methods to identify compounds (including natural ligands and other cellular compounds) that modulate the synthesis, expression or activity of either an OSCAR gene, an OSCAR gene product, an OSCAR polypeptide or a combination thereof.

As demonstrated in the Examples, infra, the OSCAR genes, gene products and polypeptides of the present invention may be characterized by their ability to modulate the maturation of osteoclast cells and, as a result, the ability to modulate growth, repair, development, resorption, degradation and homeostasis of bone tissue. Accordingly, in preferred embodiments the OSCAR nucleic acids and polypeptides of the invention, as well as antibodies directed against such OSCAR nucleic acids and polypeptides, may be used: in prognostic and diagnostic applications to identify individuals having a bone growth disorder or having a predisposition to a bone growth disorder; in methods for treating bone growth related disorders.; and in screening methods for identifying compounds (including natural ligands and other cellular compounds as well as synthetic chemical compounds) that modulate the maturation and/or activity of osteoclast, and for identifying compounds (including natural ligands and other cellular compounds, as well as synthetic chemical compounds) that modulate the growth, repair development, resorption, degradation or homeostasis of bone.

### Diagnostic Applications

A variety of methods can be employed for the diagnostic and prognostic evaluation of bone growth associated disorders such as osteopetrosis and osteporosis, and for the identification of subjects having a predisposition to such disorders. These methods utilize reagents such as the OSCAR nucleic acids and polypeptides described supra (including fragments, chimeras and fusions thereof), as well as antibodies directed against these polypeptides. For example, such reagents may be used specifically for: (1) the detection of duplications or deletions of an OSCAR gene in a cell, the presence of OSCAR gene mutations, or the detection of either over- or under-expression of an OSCAR gene product (e.g., an OSCAR mRNA) relative to expression in an unaffected state (i.e., in a subject not having or predisposed to having a bone growth associated disorder); (2) the detection of either an over- or an under-abundance of an OSCAR gene product relative to abundance in an unaffected state; and (3) the detection of an aberrant OSCAR gene product activity relative to the unaffected state.

In preferred embodiments, such reagents can be used to diagnose a bone growth related disorder such as osteopetrosis or osteoporosis, or to assess a subject's predisposition to developing a bone growth related disorder.

In preferred embodiments, the methods described herein are performed using pre-packaged diagnostic kits. Such kits may comprise at least one specific OSCAR nucleic acid or an OSCAR specific antibody reagent of the invention. The kit and any reagent(s) contained therein can be used, for example in a clinical setting, to diagnose patients exhibiting abnormalities, such as a bone growth related disorder (for example, osteopetrosis or osteoporosis).

A sample comprising a nucleated cell (of any cell type) from an individual may be used in such diagnostic methods as a starting source for genomic nucleic acid and to detect mutations of an OSCAR gene. A sample comprising a cell of any cell type or tissue of any tissue type in which an OSCAR gene is expressed may also be used in such diagnostic methods, e.g., for detection of OSCAR gene expression or of OSCAR gene products (such as OSCAR proteins) as well as for identifying cells, particularly osteoclast cells, that express an OSCAR gene or an OSCAR gene product. For example, in preferred embodiments, the expression of an OSCAR gene or an OSCAR gene product by a cell indicates that the cell is an osteoclast cell.

Detection of OSCAR nucleic acids. For the detection of OSCAR mutations or to assay levels of OSCAR nucleic acid sequences in a sample, a variety of methods may be employed. For example, mutations within an OSCAR gene may be detected by utilizing a number of techniques known in the art and with nucleic acid derived from any nucleated cell. The nucleic acid may be isolated according to standard nucleic acid preparation procedures that are already well known to those of skill in the art.

OSCAR nucleic acid sequences may be used in hybridization or amplification assays of such biological samples to detect abnormalities involving OSCAR gene structure. Exemplary abnormalities that can be detected in such methods include point mutations, single nucleotide polymorphisms (SNPs), insertions, deletions, inversions, translocations and chromosomal rearrangements. Exemplary assays that can be used to detect these abnormalities include Southern analyses, fluorescence in situ hybridization (FISH) single-stranded conformational polymorphism analyses (SSCP) and polymerase chain reaction (PCR) analyses.

As an example, and not by way of limitation, diagnostic methods for the detection of OSCAR gene-specific mutations can involve contacting and incubating nucleic acids (including recombinant DNA molecules, clones genes or degenerate variants thereof) obtained from a sample with one or more labeled nucleic acid reagents, such as recombinant OSCAR DNA molecules, cloned genes or degenerate variants thereof, under conditions favorable for the specifically annealing or hybridization of these reagents to their complementary sequences in the sample nucleic acids. Preferably, the lengths of these nucleic acid reagents are at least 15 to 30 nucleotides. After incubation, all non-annealed or non-hybridized nucleic acids are removed. The presence of nucleic acids that have hybridized, if any such molecules exist, is then detected and the OSCAR gene sequences to which the nucleic acid reagents have annealed may be compared to the annealing pattern expected from a normal (i.e., a wild-type) OSCAR gene sequence in order to determine whether an OSCAR gene mutation is present.

In a preferred embodiment of such a detection scheme, the nucleic acid from the cell type or tissue of interest may be immobilized, for example, to a solid support such as a membrane or a plastic surface (for example, on a microtiter plate or on polystyrene beads). After incubation, non-annealed, labeled OSCAR nucleic acid reagents may be easily removed and detection of the remaining, annealed, labeled OSCAR nucleic acid reagents may be accomplished using standard techniques that are well-known in the art.

Alternative diagnostic methods for the detection of OSCAR gene specific nucleic acids in patient samples or in other cell sources may involve their amplification, e.g., by PCR (see, for example, the experimental embodiment taught in U.S. Pat. No. 4,683,202) followed by detection of the amplified molecules using techniques that are well known to those of skill in the art. The resulting amplified sequences may be compared to those that would be expected if the nucleic acid being amplified contained only normal copies of an OSCAR gene in order to determine whether an OSCAR mutation is present in the samples

Other well known genotyping techniques may also be used to identify individuals carrying OSCAR mutations. Such techniques include, for example, the use of restriction fragment length polymorphisms (RFLPs). Other methods for analyzing DNA polymorphisms may be used to identify OSCAR mutations capitalize on the presence of variable numbers of short tandemly repeated DNA sequences between the restriction enzyme sites. For example, U.S. Pat. No. 5,075,217 describes a DNA marker based on length polymorphisms in blocks of short tandem repeats. The average separation of such blocks is estimated to be 30 to 70 kb. Markers that are so closely spaced exhibit a high frequency of co-inheritance and are extremely useful in the identification of genetic mutations, including for example mutations within the OSCAR gene, as well as for the diagnosis of diseases and disorders related to genetic mutations, e.g., within an OSCAR gene.

The diagnostic and prognostic methods of the invention also include methods for assaying the level of OSCAR gene expression. For example, RNA from a cell type or tissue, such as osteoclast cells, that is known or suspected to express the OSCAR gene may be isolated and tested utilizing hybridization or PCR techniques such as those described supra. The isolated cells may be, for example, cell derived from a cell culture or from a patient. The analysis of cells taken from a cell culture may be useful, e.g., to test the effect of compounds on the expression of an OSCAR gene, or alternatively, to verify that the cells are ones of a particular cell type that expresses an OSCAR gene. For instance, the Examples, infra, demonstrate that the OSCAR gene is specifically expressed in osteoclast cells. Thus, methods for assaying the level of OSCAR gene expression are particularly useful to determining whether cells (derived from a cell culture or from an individual such as a patient) are osteoclast cells.

In one preferred embodiment of such a detection scheme, a cDNA molecule is synthesized from an RNA molecule of interest (e.g., by reverse transcription). A sequence within the cDNA may then be used as a template for a nucleic acid amplification reaction such as PCR. Nucleic acid reagents used as synthesis intitation reagents (e.g., primers) in the reverse transcription and amplification steps of such an assay are preferably chosen from the OSCAR nucleic acid sequences described herein or are fragments thereof. Preferably, the nucleic acid reagents are at least about 9 to 30 nucleotides in length. The amplification may be performed using, e.g., radioactively labeled or fluorescently labeled nucleotides, for detection. Alternatively, enough amplified product may be made such that the product can be visualized by standard ethidium bromide or other staining methods.

OSCAR gene expression assays of the invention may also be performed in situ (i.e., directly upon tissue sections of patient tissue, which may be fixed and/or frozen), thereby eliminating the need of nucleic acid purification. OSCAR nucleic acid reagents may be used as probes or as primers for such in situ procedures (see, for example, Nuovo, PCR In Situ Hybridization: Protocols And Application, 1992, Raven Press, New York). Alternatively, if a sufficient quantity of the appropriate cells can be obtained, standard Northern analysis can be performed to determine the level of OSCAR gene express by detecting levels of OSCAR mRNA.

Detection of OSCAR gene products. The diagnostic and prognostic methods of the invention also include ones that comprise detecting levels of an OSCAR protein or other OSCAR polypeptide and including functionally conserved variants and fragments thereof. For example, antibodies directed against unimpaired, wild-type or mutant OSCAR gene products or against functionally conserved variants or peptide fragments of an OSCAR gene product can be used as diagnostic and prognostic reagents for bone growth related disorders such as osteopetrosis and osteoporosis. Such reagents may be used, for example, to detect abnormalities in the level of OSCAR gene product synthesis or expression, or to detect abnormalities in the structure, temporal expression or physical location of an OSCAR gene product. Antibodies and immunoassay methods such as those described herein below also have important in vitro applications for assessing the efficacy of treatments for bone growth related disorders like osteopetrosis and osteoporosis. For example, antibodies, or fragments of antibodies, can be used in screens of potentially therapeutic compounds in vitro to ascertain a compound's effects on OSCAR gene expression and OSCAR polypeptide production. Compounds that may have beneficial effects on an OSCAR associated disorder can be identified and a therapeutically effective dose for such compounds may be determined using such assays.

In vitro immunoassays can also be used to assess the efficacy of cell-based gene therapy for an OSCAR associated disorder. For example, antibodies directed against OSCAR polypeptides may be used in vitro to determine the level of OSCAR gene or polypeptide expression achieved in cells genetically engineered to produce an OSCAR polypeptide. Such methods may be used to detect intracellular OSCAR gene products, preferably using cell lysates or extracts, to detect expression of OSCAR gene products of cell surfaces, or to detect OSCAR gene products secreted into the cell culture media. Such an assessment can be used to determine the number of transformed cells necessary to achieve therapeutic efficacy in vivo, as well as optimization of the gene replacement protocol.

Generally the tissue or cell types analyzed using such methods will include ones, such as osteoclast, that are known to express an OSCAR gene product. Protein isolation methods such as those described by Harlow & Lane (Antibodies: A Laboratory Manual, 1988, Cold Spring Harbor Laboratory Press, Cold Spring Harbor, N.Y.) may be employed. The isolated cells may be cells derived from cell culture or from an individual (e.g., a patient suspected of having an OSCAR associated disorder or suspected of having a propensity for an OSCAR associated disorder).

As one example, antibodies or fragments of antibodies may be used to detect the presence of an OSCAR gene product, a variant of an OSCAR gene product or fragments thereof, for example, by immunofluorescence techniques employing a fluorescently labeled antibody coupled with light microscopic, flow cytometric or fluorimetric detection methods. Such techniques are particularly preferred for detecting OSCAR gene products on the surface of cells.

Antibodies or fragments thereof may also be employed histologically, for example in immunofluorescence or immunoelectron microscopy techniques, for in situ detection of an OSCAR gene product. In situ detection may be accomplished by removing a histological specimen (e.g., a tissue sample) from a patient and applying thereto a labeled antibody of the present invention or a fragment of such an antibody. The antibody or antibody fragment is preferably applied by overlaying the labeled antibody or antibody fragment onto a biological sample. Through the use of such a procedure, it is possible to detect, not only the presence of an OSCAR gene product, but also the gene product's distribution in the examined tissue. A wide variety of histological methods that are well known in the art (for example, staining procedures) can be readily modified by those skilled in the art without undue experimentation to achieve such in situ detection.

Immunoassays for OSCAR gene products will typically comprise incubating a biological sample (for example, a biological fluid, a tissue extract, freshly harvested cells or cell lysates) in the presence of a detectably labeled antibody that is capable of specifically binding an OSCAR gene product (including, for example, a functionally conserved variant or a peptide fragment thereof). The bound antibody may then be detected by any of a number of techniques well known in the art.

### Screening Assays

Using screening assays described herein below, it is also possible to identify compounds that bind to or otherwise interact with an OSCAR gene product, including intracellular compounds (for example, proteins or portions of proteins) that interact with an OSCAR gene product, natural and synthetic ligands for an OSCAR gene product, compounds that interfere with the interaction of an OSCAR gene product with other compounds (for example, with a natural ligand or intracellular compound), and compounds that modulate the activity of an OSCAR gene (for example, by modulating the level of OSCAR gene expression), or the activity (for example, the bioactivity) of an OSCAR polypeptide or other OSCAR gene products. For example, the screening assays described here may be used to identify compounds that bind to a promoter or other regulatory sequence of an OSCAR gene, and so may modulate the level of OSCAR gene expression (see, e.g., Platt, J. Biol. Chem. 1994, 269:28558-28562).

Classes of compounds that may be identified by such screening assays include, but are not limited to, small molecules (e.g., organic or inorganic molecules which are less than about 2 kd in molecular weight, are more preferably less than about 1 kd in molecular weight, and/or are able to cross the blood-brain barrier or gain entry into an appropriate cell and affect expression of an OSCAR gene, of some gene involved in an OSCAR regulatory pathway) as well as macromolecules (e.g., molecules greater than about 2 kd in molecular weight). Compounds identified by these screening assays may also include peptides and polypeptides. For example, soluble peptides, fusion peptides members of combinatorial libraries (such as ones described by Lam et al., Nature 1991, 354:82-84; and by Houghten et al., Nature 1991, 354:84-86); members of libraries derived by combinatorial chemistry, such as molecular libraries of D- and/or L-configuration amino acids; phosphopeptides, such as members of random or partially degenerate, directed phosphopeptide libraries (see, e.g., Songyang et al., Cell 1993, 72:767-778); antibodies, including but not limited to polyclonal, monoclonal, humanized, anti-idiotypic, chimeric, or single chain antibodies; antibody fragments, including but not limited to FAb, F(ab′)₂, FAb expression library fragments and epitope-binding fragments thereof).

As demonstrated in the Examples presented infra, the OSCAR gene product modulates the maturation and activity of osteoclast cells and, moreover, compounds such a ligands to an OSCAR gene product have the ability to modulate activity of OSCAR gene products, thereby modulating the maturation and/or activity of osteoclast cells. Thus, compounds that are identified in the screening assays described herein may be useful for modulating the activity of osteoclast cells and, in particular, for modulating the growth, repair development, degradation, resorption, repair or homeostasis of bone tissue. Accordingly, compounds identified by the screening methods described here may also be useful for treating bone growth related disorders (including, for example, osteopetrosis and osteoporosis), for example by modulating the activity of osteoclast cells and/or by modulating the growth, repair, development, resorption, degradation, repair or homeostasis of bone tissue.

Assays for binding compounds. In vitro systems can be readily designed to identify compounds capable of binding the OSCAR gene products of the present invention. Such compounds can be useful, for example, in modulating the activity of a wild-type OSCAR gene product or, alternatively, to modulate the activity of a mutant or other variant OSCAR gene product.

Generally, such screening assays involve preparation of a reaction mixture comprising an OSCAR gene product and a test compound under conditions and for a time sufficient to allow the two compounds to interact (e.g., bind), thereby forming a complex that may be detected. The assays may be conducted in any of a variety of different ways. For example, one embodiment comprises anchoring an OSCAR polypeptide or a test compound onto a solid phase and detecting complexes of the OSCAR polypeptide and the test compound that are on the solid phase at the end of the reaction and after removing (e.g., by washing) unbound compounds. For example, in one preferred embodiment of such a method, an OSCAR gene product may be anchored onto a solid surface and a labeled compound (e.g., labeled according to any of the methods described supra) is contacted to the surface. After incubating the test compound for a sufficient time and under sufficient conditions that a complex may form between the OSCAR gene product and the test compound, unbound molecules of the test compound are removed from the surface (e.g, by washing) and labeled molecules which remain are detected.

In another, alternative embodiment, molecules of one or more different test compounds are attached to the solid phase and molecules of a labeled OSCAR polypeptide may be contacted thereto. In such embodiment, the molecules of different test compounds are preferably attached to the solid phase at a particular location on the solid phase so that test compounds that bind to an OSCAR polypeptide may be identified by determining the location of bound OSCAR polypeptides on the solid phase or surface.

Assays for compounds that interact with OSCAR. Any of a variety of known methods for detecting protein-protein interactions may also be used to detect and/or identify proteins that interact with an OSCAR gene product. For example, co-immunoprecipitation, cross-linking and co-purification through gradients or chromatographic columns as well as other techniques known in the art may be employed. Proteins which may be identified using such assays include, but are not limited to, extracellular proteins, such as OSCAR specific ligands, as well as intracellular proteins such as signal transducing proteins.

As an example, and not by way of limitation, an expression cloning assay may be used to identify OSCAR specific ligands and other proteins that specifically interact with an OSCAR gene product. In such assays, a cDNA expression library may be generated from any cell line that expresses an OSCAR specific ligand (for example, osteoblast cells, embryonic fibroblast cells, NIH cells, 3T3 cells, ST2 cells, Mlg cells, UMR106 cells, HEK293 cells, HEK293T cells, hFOB1.19 cells and monkey COS-1 cells). Clones from such an expression library may then be transfected or infected into cells, such as a B cell lymphoma line (e.g., CH12 cells, A20.25 cells or LBB1 cells) that do not normally express an OSCAR specific ligand. Cells that are transfected with a clone that encodes an OSCAR specific ligand may then express this gene product, and can be identified and isolated using standard techniques such as FACS or using magnetic beads that have an OSCAR polypeptide (for example, an OSCAR-Fc fusion polypeptide) attached thereto.

Alternatively, an OSCAR specific ligand may be isolated from a cell line, including any of the OSCAR-L expressing cell lines recited above, using immunoprecipitation techniques that are well known in the art.

OSCAR specific ligands may also be isolated using any of the screening assays discussed, supra for identifying OSCAR binding compounds. For example, an OSCAR-Fc fusion polypeptide may be bound or otherwise attached to a solid surface, and a labeled compound (e.g., a candidate OSCAR ligand) may be contacted to the surface for a sufficient time and under conditions that permit formation of a complex between the OSCAR-Fc fusion polypeptide and the test compound. Unbound molecules of the test compound can then be removed from the surface (e.g., by washing), and labeled compounds that remain bound can be detected.

Once so isolated, standard techniques may be used to identify any protein detected in such assays. For example, at least a portion of the amino acid sequence of a protein that interacts with the OSCAR gene product can be ascertained using techniques well known in the art, such as the Edman degradation technique (see, e.g., Creighton, 1983, Proteins: Structures and Molecular Principles, W.H. Freeman&Co., New York, pages 34-49).

Once such proteins have been identified, their amino acid sequence may be used as a guide for the generation of oligonucleotide mixtures to screen for gene sequences encoding such proteins; e.g., using standard hybridization or PCR techniques described supra. See, for example, Ausubel supra; and PCR Protocols: A Guide to Methods and Applications, Innis et al., eds., Academic Press, Inc., New York (1990) for descriptions of techniques for the generation of such oligonucleotide mixtures and their use in screening assays.

Other methods are known in the art which result in the simultaneous identification of genes that encode a protein that interacts with an OSCAR polypeptide. For example, expression libraries may be probed with a labeled OSCAR polypeptide.

As another example and not by way of limitation, the two-hybrid system may be used to detect protein interactions with an OSCAR gene product in vivo. Briefly, utilizing such a system, plasmids may be constructed which encode two hybrid proteins: one of which preferably comprises of the DNA-binding domain of a transcription activator protein fused to an OSCAR gene product. The other hybrid protein preferably comprises an activation domain of the transcription activator protein used in the first hybrid, fused to _ an unknown protein that is encoded by a cDNA recombined into the plasmid library as part of a cDNA library. Both the DNA-binding domain fusion plasmid and the cDNA library may be co-transformed into a strain of Saccharomyces cerevisiae or other suitable organism which contains a reporter gene (for example, HBS, lacZ, 1IS3 or GFP). Preferably, the regulatory region of this reporter gene comprises a binding site for the transcription activator moiety of the two hybrid proteins. In such a two-hybrid system, the presence of either of the two hybrid proteins alone cannot activate transcription of the reporter gene. Specifically, the DNA-binding domain hybrid protein cannot activate transcription because it cannot localize to the necessary activation function. Likewise, the activation domain hybrid protein cannot activate transcription because it cannot localize to the DNA binding site on the reporter gene. However, interaction between the two hybrid proteins, reconstitutes that functional transcription activator protein and results in expression of the reporter gene. Thus, in a two-hybrid system such as the one described here in detail, an interaction between an OSCAR polypeptide (i e., the OSCAR polypeptide fused to the transcription activator's DNA binding domain) and a test polypeptide (i.e., a protein fused to the transcription activator's DNA binding domain) may be detected by simply detecting expression of a gene product of the reporter gene. cDNA libraries for screening in such two-hybrid and other assay may be made according to any suitable technique known in the art. As a particular and non-limiting example, cDNA fragments may be inserted into a vector so that they are translationally fused to the transcriptional activation domain of GAL4, and co-transformed along with a “bait” OSCAR-GAL4 fusion plasmid into a strain of Saccharomyces cerevisiae or other suitable organism that contains a 1-US3 gene driven by a promoter that contains a GAL4 activation sequence. A protein from this cDNA library, fused to the GAL4 transcriptional activation domain, which interacts with the OSCAR polypeptide moiety of the OSCAR-GAL4 fusion will reconstitute and active GAL4 protein and can thereby drive expression of the HIS3 gene. Colonies that express the HIS3 gene may be detected by their growth on petri dishes containing semi-solid agar based media lacking histidine. The cDNA may then be purified from these strains, sequenced and used to identify the encoded protein which interacts with the OSCAR polypeptide.

Once compounds have been identified which bind to an OSCAR gene product of the invention, the screening methods described in these methods may also be used to identify other compounds (e.g., small molecules, peptides and proteins) which bind to these binding compounds. Such compounds may also be useful to modulating OSCAR-related bioactivities, for example by binding to a natural OSCAR ligand or binding partner, and preventing its interaction with an OSCAR gene product. For instance, these compounds could be tested for their ability to inhibit the binding of OSCAR-Fc to cell lines which express OSCAR-L (see, supra).

Assays for compounds that interfere with air OSCAR-ligand interaction. The Examples presented infra demonstrate that an OSCAR gene product of the invention may interact with one or more molecules (i.e., ligands) in vivo. Compounds that disrupt or otherwise interfere with this binding interaction are useful in modulating activity of an OSCAR gene product, as is also demonstrated in the Examples infra. In particular, such compounds modulate the maturation or activity of osteoclast cells, which, in turn, is implicated in modulating the growth, repair, development, resorption, degradation or homeostasis of bone tissue, or for treating bone growth related disorders.

Such compounds include, but are not limit to, compounds identified according to the screening assays described supra, for identifying compounds that bind to an OSCAR gene product, including any of the numerous exemplary classes of compounds described therein.

In general, assays for identifying compounds that interfere with the interaction between an OSCAR gene product and a binding partner (e.g., a ligand) involve preparing a test reaction mixture that contains the OSCAR gene product and its binding partner under conditions and for a time sufficient for the OSCAR gene product and its binding partner to bind and form a complex. In order to test a compound for inhibitory activity (i.e., for the ability to inhibit formation of the binding complex or to disrupt the binding complex once formed), the test compound preferably is also present in the test reaction mixture. In one exemplary embodiment, the test compound may be initially included in the test reaction mixture with the OSCAR gene product and its binding partner. Alternatively, however, the test compound may be added to the test reaction mixture at a later time, subsequent to the addition of the OSCAR gene product and its binding partner. In preferred embodiments, one or more control reaction mixtures, which do not contain the test compound, may also be prepared. Typically, a control reaction mixture will contain the same OSCAR gene product and binding partner that are in the test reaction mixture, but will not contain a test compound. A control reaction mixture may also contain a placebo, not present in the test reaction mixture, in place of the test compound. The formation of a complex between the OSCAR gene product and the binding partner may then be detected in the reaction mixture. The formation of such a complex in the absence of the test compound (e.g., in a control reaction mixture) but not in the presence of the test compound, indicates that the test compound is one which interferes with or modulates the interaction of an OSCAR polypeptide and a binding partner.

Such assays for compounds that modulate the interaction of an OSCAR gene product and a binding partner may be conducted in a heterogenous format or, alternatively, in a homogeneous format. Heterogeneous assays typically involve anchoring either an OSCAR gene product or a binding partner onto a solid phase and detecting compounds anchored to the solid phase at the end of the reaction. Thus, such assays are similar to the solid phase assays described supra for detecting and/or identifying OSCAR nucleic acids and gene products and for detecting or identifying OSCAR binding partners. Indeed, those skilled in the art will recognize that many of the principles and techniques described above for those assays may be modified and applied without undue experimentation in the solid phase assays described here, for identifying compounds that modulate interaction(s) between and OSCAR gene product and a binding partner.

Regardless of the particular assay used, the order to which reactants are added to a reaction mixture may be varied; for example, to identify compounds that interfere with the interaction of an OSCAR gene product with a binding partner by competition, or to identify compounds that disrupt a preformed binding complex. Compounds that interfere with the interaction of an OSCAR gene product with a binding partner by competition may be identified by conducting the reaction in the presence of a test compound. Specifically, in such assays a test compound may be added to the reaction mixture prior to or simultaneously with the OSCAR gene product and the binding partner. Test compounds that disrupt preformed complexes of an OSCAR gene product and a binding partner may be tested by adding the test compound to a reaction mixture after complexes have been formed.

The screening assays described herein may also be practiced using peptides or polypeptides that correspond to portions of a full length OSCAR polypeptide or protein, or with fusion proteins comprising such peptide or polypeptide sequences. For example, screening assays for identifying compounds the modulate interactions of an OSCAR polypeptide with a binding partner may be practiced using peptides or polypeptides corresponding to particular regions or domains of a full length OSCAR polypeptide that bind to a binding partner (e.g., ligand “binding sites”). For example, in one embodiment screening assays may be carried out using polypeptides (or fusions thereof) that comprise an amino acid sequence corresponding to extracellular domain of a full length OSCAR polypeptide (e.g., comprising the sequence of amino acid residues 1-228 of the OSCAR polypeptide set forth in SEQ ID NO:3).

A variety of methods are known in the art that may be used to identify specific binding sites of an OSCAR polypeptide. For example, binding sites may be identified by mutating an OSCAR gene and screening for disruptions of binding as described above. A gene encoding the binding partner may also be mutated in such assays to identify mutations that compensate for disruptions from the mutation to the OSCAR gene. Sequence analysis of these mutations can then reveal mutations that correspond to the binding region of the two proteins.

In an alternative embodiment, a protein (e.g., an OSCAR protein or a protein binding partner to an OSCAR protein) may be anchored to a solid surface or support using the methods described herein above. Another labeled protein which binds to the protein anchored to the solid surface may be treated with a proteolytic enzyme, and its fragments may be allowed to interact with the protein attached to the solid surface, according to the methods of the binding assays described supra. After washing, short, labeled peptide fragments of the treated protein may remain associated with the anchored protein. These peptides can be isolated and the region of the full length protein from which they are derived may be identified by the amino acid sequence.

In still other embodiments, compounds that interfere with an OSCAR-ligand interaction may also be identified by screening for compounds that modulate binding of an OSCAR polypeptide (for example, an OSCAR-Fc fusion polypeptide) to cells that express an OSCAR specific ligand, such as osteoblast cells, embryonic fibroblast cells, NIH cells, 3T3 cells, ST2 cells, Mlg cells, UMR106 cells, HEK293 cells, HEK293T cells, hFOB1.19 cells and COS-1 cells.

### Therapeutic Methods and Pharmaceutical Preparations

OSCAR nucleic acid molecules, polypeptides and antibodies of the present invention may be used, for example, to modulate the maturation and activity of osteoclast cells. In addition, compounds that bind to an OSCAR nucleic acid or polypeptides of the invention, compounds that modulate OSCAR gene expression, and compounds that interfere with or modulate binding of an OSCAR nucleic acid or polypeptide with a binding compound (e.g., with a natural ligand) may be useful, e.g., in methods for modulating the maturation or activity of osteoclast cells. Accordingly, such compounds may also be used to modulate processes associated with osteoclast cell activity, for example the growth, repair, development, resorption, degradation and homeostasis of bone tissue. Such methods may be particularly useful for treating bone growth related disorders, such as osteoporosis, osteopetrosis and the like.

For example, compounds that bind to an OSCAR gene product of the invention (for example, OSCAR ligands), may increase OSCAR activity, stimulate the maturation of osteoclast cells and thereby increase osteoclast cell related activities. Such compounds may be used, therefore, to treat conditions in which activation of osteoclast activity may be desirable. For example, because osteoclast cells are ones that reabsorb calcified bone matrix, compounds that increase OSCAR activity and induce the maturation of osteoclast cell are useful for treating bone growth related disorders, such as osteopetrosis, that are associated with abnormally high or elevated bone mass. Alternatively, compounds that decrease OSCAR activity, for example by interfering with binding interactions between an OSCAR gene product and a ligand, may reduce osteoclast cell maturation and osteoclast cell related activities. These compounds may therefore be used to treat conditions in which reduced osteoclast cell activity may be desirable. For example, compounds that decrease OSCAR activity can be used to treat bone growth related disorders, such as osteoporosis, that are associated with abnormally low or decreased bone mass..

Such methods may be used to determine whether a compound actually increases or decreases the number of osteoclast cells, e.g., in a tissue sample. Accordingly, these methods may be used to monitor whether a particular treatment is producing a desired affect on osteoclast cell activity.

Alternatively, the effectivity of a treatment may be ascertained by monitoring the bone mass of an individual (e.g., in an animal model or in a patient) and determining whether bone mass has increased or decreased as a result of the therapy.

Inhibitory Approaches. Methods for modulating osteoclast cell maturation or activity may simply comprise administering one or more compounds that modulate expression of an OSCAR gene, synthesis of an OSCAR gene product or OSCAR gene product activity so the osteoclast cell maturation or activity is modulated (e.g., increased or decreased). Likewise, methods for modulating (e.g., increasing or decreasing) bone growth, repair, development, resorption, degradation or homeostasis may simply comprise administering one or more compounds that modulate expression of an OSCAR gene, synthesis of an OSCAR gene product or OSCAR gene product activity. Preferably, these one or more compounds are administered until bone growth, repair, development, resorption, degradation or homeostasis is modulated as desired.

Among the compounds that may exhibit an ability to modulate the activity, expression or synthesis of an OSCAR nucleic acid are antisense, ribozyme and triple-helix molecules. Such molecules may be designed to reduce or inhibit wild-type OSCAR nucleic acids and polypeptides or, alternatively, may target mutant OSCAR nucleic acids or polypeptides.

Antisense RNA and DNA molecules act to directly block the translation of mRNA by hybridizing to target mRNA molecules and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

A sequence that is “complementary” to a portion of a nucleic acid refers to a sequence having sufficient complementarity to be able to hybridize with the nucleic acid and form a stable duplex. The ability of nucleic acids to hybridize will depend both on the degree of sequence complementarity and the length of the antisense nucleic acid. Generally, however, the longer the hybridizing nucleic acid, the more base mismatches it may contain and still form a stable duplex (or triplex in triple helix methods). A tolerable degree of mismatch can be readily ascertained, e.g., by using standard procedures to determine the melting temperature of a hybridized complex.

In one preferred embodiment, oligonucleotides complementary to non-coding regions of an OSCAR gene may be used in an antisense approach to inhibit translation of endogenous OSCAR mRNA molecules. Antisense nucleic acids are preferably at least six nucleotides in length, and more preferably range from between about six to about 50 nucleotides in length. In specific embodiments, the oligonucleotides may be at least 10, at least 15, at least 20, at least 25 or at least 50 nucleotides in length.

It is generally preferred that in vitro studies are first performed to quantitate the ability of an antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

While antisense nucleotides complementary to the target gene coding region sequence could be used, those complementary to the transcribed, untranslated region are most preferred.

Antisense molecules are preferably delivered to cells, such as osteoclast cells, that express the target gene in vivo. A number of methods have been developed for delivering antisense DNA or RNA to cells. For example, antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

Preferred embodiments achieve intracellular concentrations of antisense nucleic acid molecules which are sufficient to suppress translation of endogenous mRNAs. For example, one preferred approach uses a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector, as set forth above, can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in the particular cell type (for example in a mammalian osteoclast cell, such as a human osteoclast cell). For example, any of the promoters discussed supra in connection with the expression of recombinant OSCAR nucleic acids can also be used to express an OSCAR antisense nucleic acid.

Ribozyme molecules designed to catalytically cleave target gene mRNA transcripts can also be used to prevent translation of target gene mRNA and, therefore, expression of target gene product (see, e.g., International Publication No. WO 90/11364; Sarver, et al., Science 1990, 247:1222-1225).

Ribozymes are enzymatic RNA molecules capable of catalyzing the specific cleavage of RNA (for a review, see Rossi, Current Biology 1994, 4:469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246.

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, Nature 1988, 334:585-591.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., Science 1984, 224:574-578; Zaug and Cech, Science 1986, 231:470-475; Zaug et al., Nature 1986, 324:429-433; International Patent Publication No. WO 88/04300; Been and Cech, Cell 1986, 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the target gene.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes unlike antisense molecules, are catalytic, a lower intracellular concentration is required for efficacy. Such constructs can be introduced to cells using any of the vectors described supra.

Endogenous target gene expression can also be reduced by inactivating or “knocking out” the target gene or its promoter using targeted homologous recombination (e.g., see Smithies, et al., Nature 1985, 317:230-234; Thomas and Capecchi, Cell 1987, 51:503-512; and Thompson et al., Cell 1989, 5:313-321). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (see generally, Helene, Anticancer Drug Des. 1991, 6:569-584; Helene, et al., Ann. N.Y. Acad. Sci. 1992, 660:27-36; and Maher, Bioassays 1992, 14:807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles that the possibility may arise wherein the concentration of normal target gene product present may be lower than is necessary for a normal phenotype. In such cases, to ensure that substantially normal levels of target gene activity are maintained, therefore, nucleic acid molecules that encode and express target gene polypeptides exhibiting normal target gene activity may, be introduced into cells via gene therapy methods such as those described, below, that do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Gene Therapy. In instances wherein a disorder results from an OSCAR gene mutation, treatment methods may comprise supplying an individual with a wild type OSCAR nucleic acid molecule or one which encodes an OSCAR polypeptide having normal bioactivity so that symptoms of the disorder are ameliorated.

Alternatively, in instances wherein a disorder results from an OSCAR gene mutation, treatment may comprise engrafting or supplying an individual with a cell, such as an osteoclast or fibroblast cell, which has been modified to expresses a wild-type OSCAR gene product or an OSCAR gene product having normal bioactivity so that symptoms of the disorder are ameliorated.

Any of the methods for gene therapy available in the art can be used according to the present invention. For general reviews of the methods of gene therapy, see Goldspiel et al., Clinical Pharmacy 1993, 12:488-505; Wu and Wu, Biotherapy 1991, 3:87-95; Tolstoshev, Ann. Rev. Pharmacol. Toxicol. 1993, 32:573-596; Mulligan, Science 1993, 260:296-932; Morgan and Anderson, Ann. Rev. Biochem. 1993, 62:191-217; and May, TIBTECH 1993, 11:155-215). In particular, any of the viral and non-viral vectors described supra for expression OSCAR nucleic acids in cell may be used in these gene therapy methods.

Methods that are commonly known in the art of recombinant DNA technology may also be used in such gene therapy methods. For example, see methods described in Ausubel et al. (eds.), 1993, Current Protocols in Molecular Biology, John Wiley & Sons, New York; Kriegler, 1990, Gene Transfer and Expression: A Laboratory Manual, Stockton Press, New York; and Dracopoli et al. (eds.), 1994, Current Protocols in Human Genetics, John Wiley & Sons, New York.

In one aspect of the gene therapy methods of the invention, a therapeutic vector, including any of the expression vectors described herein, is used which comprises a nucleic acid sequence that expresses a functional OSCAR gene product in a suitable host cell. In particular, the vector preferably contains nucleic acid sequences comprising a promoter operatively linked to the coding sequence for a function OSCAR polypeptide of the invention. The promoter may be an inducible promoter, a constitutive promoter and, optionally, may be tissue-specific. In another embodiment, the vector contains a nucleic acid molecule in which an OSCAR nucleic acid sequence is flanked by regions that promote homologous recombination at a desired site in the genome, thus providing for intrachromosomal expression of an OSCAR gene product (see, for example, Koller and Smithies, Proc. Natl. Acad. Sci. U.S.A. 1989, 86:8932-8935; Zijlstra et al., Nature 1989, 342:435-438).

In embodiments wherein the vector is administered to an individual (for example, in methods to modulate osteoclast cell activities such as bone growth, repair, development, resorption, degradation or homeostasis), delivery of the vector into the individual may be either direct or indirect. Direct methods of vector delivery comprise directly exposing the individual to the vector or delivery complex. In indirect methods of delivery, cells are first transformed with the vector in vitro (for example, in a cell culture) and then transplanted into the patient. Such direct and indirect methods of delivery are also referred to as in vivo and ex vivo gene therapy methods, respectively.

The exact form and amount of nucleic acid used in such gene therapy methods will depend on the specific application, such as the particular type of disease and the severity of the desired effect, patient state, and so forth. An appropriate form and amount of nucleic acid for a particular application or therapy may be determined by one skilled in the art.

Anti-OSCAR antibody therapy. As demonstrated in the specific Examples infra, an OSCAR gene product of the present invention is expressed predominantly or exclusively in osteoclast cells. Accordingly, the therapeutic methods of the present invention also include the use of antibodies that specifically bind to an OSCAR gene product to target and transiently ablate osteoclast cells. Such methods of therapy are particularly desirable for treating diseases and disorders, such as osteopetrosis, where suppression of osteoclast-mediated bone resorption is desirable.

Any of the antibodies described supra that specifically bind to an OSCAR polypeptide of the invention may be used in such therapies. For example, therapeutic antibodies used in such methods may be full length antibodies or fragments thereof conjugated to a cytotoxic molecule (for example, a radioisotope or a toxin, such as ricin). The antibody may then be used to specifically target cytoxicity to the target cells (i e., to osteoclast cells).

In other embodiment of these methods, the endogenous function of an antibody (i e., the function mediated by the Fc portion of the antibody) to clear target osteoclast cells, e.g., by antibody-mediated cytoxicity and the like. Such antibody-based therapies are already well known in the art.

In still other embodiments, intracellular antibodies (also referred to as “intrabodies”) may be used to regulate the activity of an OSCAR gene product. The use of intrabodies to regulate the activity of intracellular proteins is well known in the art and has been described for a number of different systems (see, e.g., Marasco, Gen Ther. 1997, 4:11; Chen et al., Hum. Gene Ther. 1994, 5:595), including (but not limited to) viral infections (see, for example, Marasco et al., Hum. Gene Ther. 1998, 9:1627) and other infectious diseases (see, e.g., Rondon et al., Annu. Rev. Microbiol. 1997, 51:257), as well as oncogenes, such as p21 (for example, see Cardinale et al., FEBS Lett. 1998, 439:197-202; and Cochet et al., Cancer Res. 1998, 58:1170-6), myb (see, Kasono et al., Biochem Biophys Res Commun. 1998, 251:124-30), erbB-2 (Graus-Porta et al., Mol Cell Biol. 1995, 15:1182-91), etc.

Pharmaceutical Preparations. Compounds that are determined to affect OSCAR gene expression or OSCAR gene product activity may be administered (e.g., to an individual) at therapeutically effective doses to modulate osteoclast cell maturation or osteoclast cell associated activities; or such compounds may be administered at therapeutically effective doses to modulate the growth, repair, development, resorption, degradation or homeostasis of bone tissue in an individual. The term therapeutically effective dose therefore refers to that amount of the compound that is sufficient to result in such modulated activities and/or in amelioration in symptoms of a bone growth related disorder such as osteoporosis and osteopetrosis.

Toxicity and therapeutic efficacy of compounds can be determined by standard pharmaceutical procedures, for example in cell culture assays or using experimental animals to determine the LD₅₀ and the ED₅₀. The parameters LD₅₀ and ED₅₀ are well known in the art, and refer to the doses of a compound that are lethal to 50% of a population and therapeutically effective in 50% of a population, respectively. The dose ratio between toxic and therapeutic effects is referred to as the therapeutic index and may be expressed as the ratio: LD₅₀/ED₅₀. Compounds that exhibit large therapeutic indices are preferred. While compounds that exhibit toxic side effects may be used. However, in such instances it is particularly preferable to use delivery systems that specifically target such compounds to the site of affected tissue so as to minimize potential damage to other cells, tissues or organs and to reduce side effects.

Data obtained from cell culture assay or animal studies may be used to formulate a range of dosages for use in humans. The dosage of compounds used in therapeutic methods of the present invention preferably lie within a range of circulating concentrations that includes the ED₅₀ concentration but with little or no toxicity (e.g., below the LD₅₀ concentration). The particular dosage used in any application may vary within this range, depending upon factors such as the particular dosage form employed, the route of administration utilized, the conditions of the individual (e.g., patient), and so forth.

A therapeutically effective dose may be initially estimated from cell culture assays and formulated in animal models to achieve a circulating concentration range that includes the IC₅₀. The IC₅₀ concentration of a compound is the concentration that achieves a half-maximal inhibition of symptoms (e.g., as determined from the cell culture assays). Appropriate dosages for use in a particular individual, for example in human patients, may then be more accurately determined using such information.

Measures of compounds in plasma may be routinely measured in an individual such as a patient by techniques such as high performance liquid chromatography (HPLC) or gas chromatography.

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may talce the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain-buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### EXAMPLES

The invention is also described by means of particular examples. However, the use of such examples anywhere in the specification is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

### Example 1

### Isolation and Characterization of the Murine OSCAR Gene

This example describes the isolation of a novel cDNA fragment encoding for an immunoglobulin (Ig)-like receptor, which is specifically expressed in osteoclasts. The example provides a novel gene and gene product herein called OSCAR.

### Materials and Methods

Preparation of osteoclasts and macrophages. Bone marrow cells were isolated from 4 to 8 week-old C57BL/6 male mice as described (Wani et al., Endocrinology 1999, 140:1927-1935). Femora and tibiae were aseptically removed. The bone ends were cut and the marrow cells were flushed out by injecting BSS solution using a sterile 31-gauge needle. To obtain a single cell suspension, the marrow cells were agitated with a plastic Pasteur pipette. After filtering with mesh, the marrow cells were treated with Gey's solution. The marrow cells were washed twice, resuspended in α-MEM containing 10% FBS, and incubated for 24 hours in M-CSF (5 ng/ml) at a density of 1×10⁶ cells/ml in a 750 ml flask. After 24 hours, the nonadherent cells were harvested and resuspended in the same media. 10 ml of the suspension (3×10⁷ cells) were added in a 100 mm petri dish for preparation of the osteoclast and macrophage cells. Human M-CSF (30 ng/ml) was added for macrophage cells, while hM-CSF (30 ng/ml), mTRANCE (1 μg/ml), and PGE2 (1 μM) were used for osteoclast cells. Cultures were fed at day 3 and the adherent cells were harvested at day 4 after washing with twice with PBS. Mature dendritic cells were generated from bone marrow precursors as described (Inaba et al., J. Exp. Med. 1992, 176:1693-1702).

Isolation of RNA from bone marrow cells. The total RNA from the osteoclast and macrophage cells was isolated directly from the culture dishes using TRIZOL (GIBCO). The polyA mRNA was isolated from total RNA using the oligotex mRNA kit (QIAGEN). The eluents of the polyA mRNA were precipitated with ethanol and resuspended in DEPC-treated distilled water. The concentration of the polyA mRNA was determined by UV spectrophotometer.

Isolation of RNA from skull and long bones. Skulls from 3 day old mice were collected, washed with PBS, and treated with TRIZOL. The long bones from 4 week old female mice were collected, frozen, crushed using a Bessman tissue pulverizer (Fisher), and treated with TRIZOL. Total RNA from the tissue samples was harvested using TRIZOL according to manufacturer's protocol (GIBCO)

Generation of a subtraction cDNA library. The polyA mRNA from the bone marrow-derived osteoclast and macrophage cells were used to prepare a subtraction cDNA library using a PCR-selected subtraction kit according to the manufacturer's protocol (CLONTECH). The cDNAs from the subtraction were directly inserted into pCR2.1 TA cloning vector (INVITROGEN). After overnight ligation at 14° C., the ligation mixture was transformed into E. coli XLIIB competent cells. These cells were plated on LB plates containing ampicillin with X-gal and IPTG. 250 white colonies were randomly picked for miniprep culture.

Identification of osteoclast specific genes. The plasmid DNA samples containing the subtracted fragments were isolated using the QIAprep spin miniprep kit (QIAGEN). After digestion with EcoRI, the DNA was separated on agarose gels, transferred to Nylon membranes (NEN), and probed with ³²P-labeled cDNA from osteoclasts and macrophages. ³²P-labelled total cDNA probes were synthesized with each total RNA using random hexamers as primers as described previously (Sambrook et al, 1989, supra).

The nylon membranes were prehybridized for 4 hours in hybridization buffer (50% formamide, 150 mM sodium phosphate, pH 6.8, 2× Denhardt's solution, 250 mM NaCl, 1% SDS, 1mM EDTA, and 10% PEG 8,000). The denatured DNA probes were added and hybridized for 16 hours. The filters were washed and autoradiographed as previously described (Sambrook et al., 1989, supra). Samples which hybridized selectively to osteoclast probes were selected for further analysis.

Northern Analysis. Northern blot analysis was performed using Northern hybridization buffer (50% formamide, 50 mM sodium phosphate, pH 6.8, 5× Denhardt's solution, 5×SSC, and 3 mg/ml sonicated salmon sperm DNA) as described (Sambrook et al., 1989, supra). The total RNA from the different cell types and tissue samples was harvested using TRIZOL according to manufacturer's protocol (GIBCO). OCL178, and full length TRAP and Cathepsin K cDNA were labeled and used as probes.

### Results and Discussion

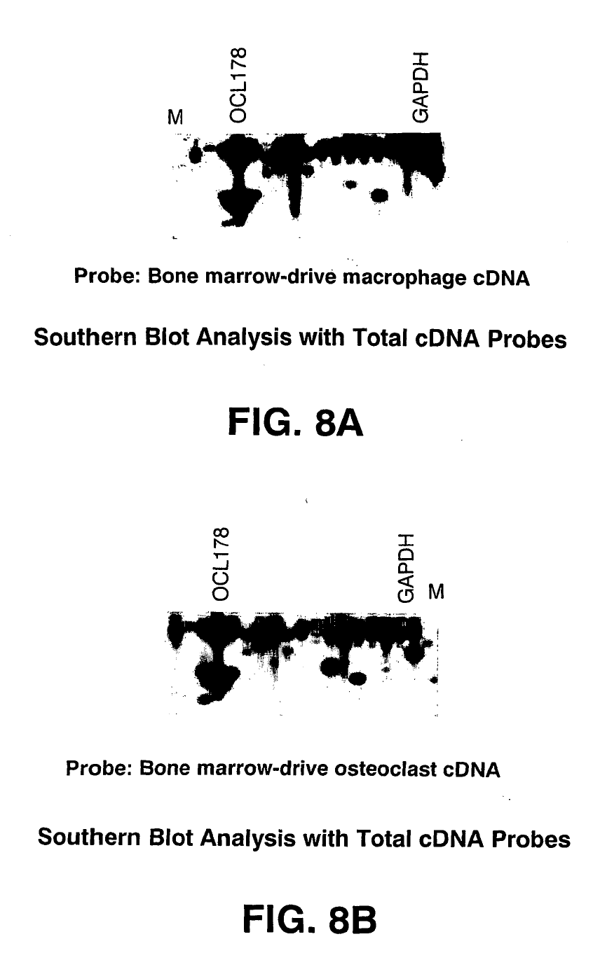

Isolation of cDNA fragments for OSCAR. Osteoclasts (OC) and macrophages (MØ) are derived from bone marrow precursor cells. Since OCs and MØs are derived from a potentially common precursor cells, we constructed a subtraction cDNA (murine OC minus MØ) library using the PCR-select subtraction kit according to the manufacturer's protocol (CLONTECH). To identify OC-specific genes, plasmid DNA containing the subtracted fragments was purified from 250 clones, digested, separated on agarose gels, transferred to Nylon membranes (NEN), and probed with ³²P-labelled cDNAs from OCs or MØs (FIG. 8). One clone, referred to as OCL178, was identified which is more highly expressed in osteoclast cells than in macrophages. This clone was selected for further analysis. The clone was determined to be a fragment of a novel gene, referred to herein as OSCAR.

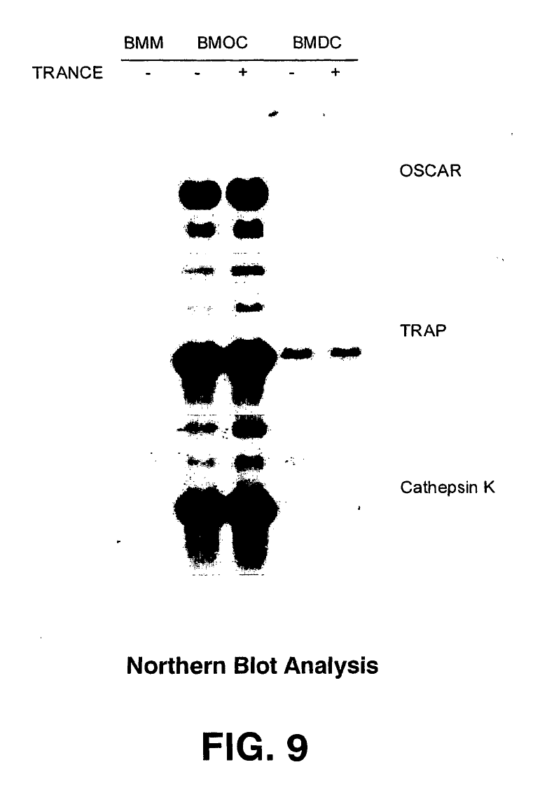

OSCAR is specifically expressed in OCs, but not in MØs or deudritic cells (DCs). To test whether OCL178 is derived from a gene specifically expressed in osteoclast cells, mRNA derived from OCs and MØs was hybridized with ³²P-labelled OCL178. As shown in FIG. 9, the OCL178 fragment detected three distinct mRNA species with apparent sizes of 4.0 kB, 1.8 kb, and 1.0 kb. Expression of OSCAR was specifically detected in bone-marrow derived OCs (BMOC), but not in bone-marrow derived MØs (BMM). Moreover, OSCAR expression was not detected in bone-marrow derived DCs (BMDCs), which are derived from the same precursor as OCs and MØs. TRAP and Cathepsin K are genes considered in the art to be osteoclast specific markers since their expression has been detected in OCs but not in MØs (see, e.g., Minkin, C., Calcif Tissue Int., 1982,34:285; Ek-Rylander, et al., Biochem J. 1997, 321:305-11; Chambers, et al., Cell Tissue Res., 1985, 241:671-675; Lacey, et al., Cell, 1998, 93:165-176). However, unlike OSCAR, expression of TRAP and/or Cathepsin K can also detected in BMDCs (see, FIG. 9). The expression of OSCAR in osteoclast cells is therefore much more specific than either TRAP or Cathepsin K, demonstrating that the OSCAR gene and its gene product are osteoclast specific markers which are improved over other markers (e.g., TRAP and Cathepsin K) known in the art.

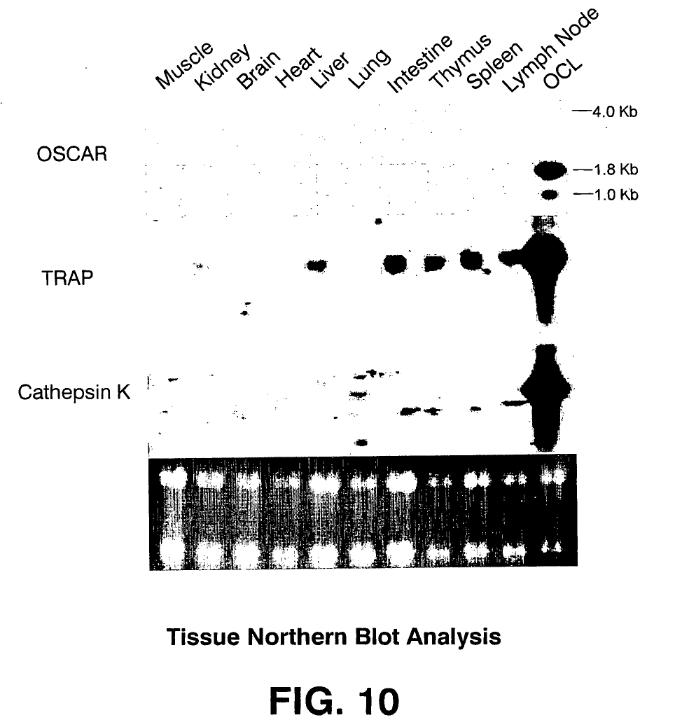

OSCAR is specifically expressed in OCs, but not in other cells. To determine the specificity of OSCAR mRNA expression, mRNA from various tissues were analyzed by Northern analysis (FIG. 10). As shown in FIG. 10, OSCAR mRNA expression is specifically detected in OCs (OCL), but not in other tissues tested; including muscle, kidney, brain, heart, liver, lung, intestine, thymus, spleen and lymph node. In comparison, TRAP or Cathepsin K mRNA, which are considered in the art to be specific-markers for OCs, can be detected in mRNA derived from other cell types (i.e., cells derived from tissues other than osteoclast). Thus, this result confirms that OSCAR expression is specific to osteoclast cells, and that the OSCAR gene and its gene product are improved osteoclast cell specific markers.

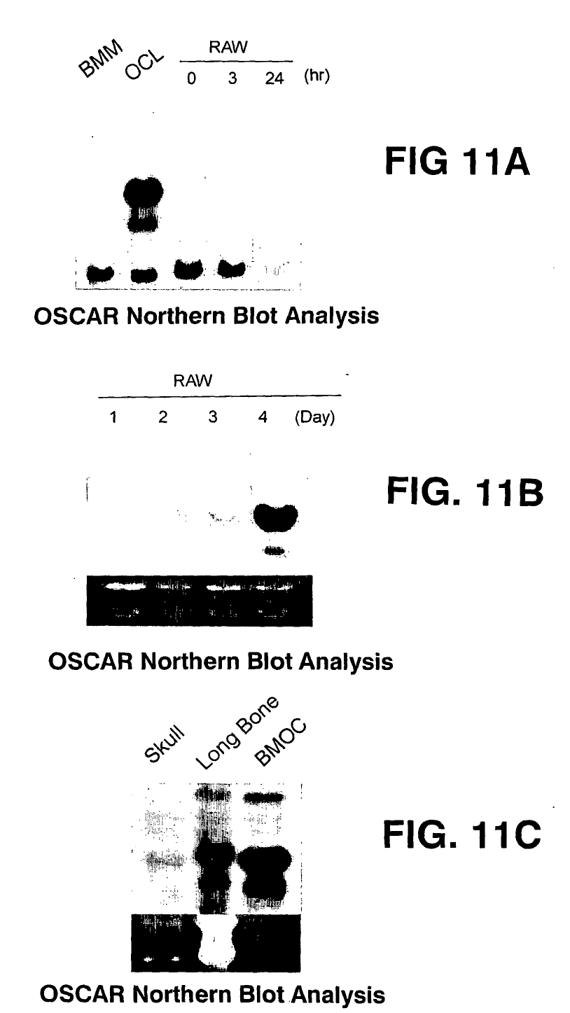

OSCAR is expressed in cells differentiated in vitro as well as in vivo. RAW264.7 cells have been shown to differentiate into osteoclast-like cells in vitro upon treatment with TRANCE (Hsu et al., Proc. Natl. Acad. Sci. U.S.A. 1999, 96:3540-3545). Northern Blot analysis, shown here in FIGS. 11A-C, demonstrate that these cells also express OSCAR within 48 hours of treatment. OSCAR expression is highest after four days, when the cells have completely differentiated.

In addition, although OSCAR expression is not detected in the various tissues described above (e.g., muscle, kidney, brain, heart, liver, lung, intestine, thymus, spleen and lymph node), OSCAR mRNA is detected in Northern Blot analysis of osteoclast rich tissues such as skull and long bones (FIG. 11C).

Thus, OSCAR is expressed in differentiated osteoclast and, further, such expression occurs regardless of whether differentiation occurs in vivo or in vitro.

### Example 2

### OSCAR Encodes a Novel Immunoglobulin (Ig)-Like Receptor

This example describes the isolation and characterization of cDNA molecules that contain sequences encoding full length, murine OSCAR polypeptides.

### Materials and Methods

Generation of mouse cDNA library. A mouse cDNA library was generated using polyA mRNA from bone marrow-derived mature osteoclast cells according to manufacture's protocol (STRATAGENE). The full length OSCAR cDNAs were isolated from this library by screening using the OCL178 insert as described (Sambrook et al., 1989, supra).

Amino acid sequence analysis. Full-length murine OSCAR amino acid sequences were used to search for homologous protein sequences in the NCBI protein database. Searches were conducted using the BLAST family of algorithms (Altschul et al., Nucleic Acids Res. 1997, 25:3389-3402; Altschul et al., 1990, J. Mol. Biol. 1990, 215:403-410) with default parameter values.

### Results and Discussion

OCL178 was used to screen a cDNA library derived from murine bone-marrow osteoclast cells. Clones corresponding to the 1.8 kb and 1.0 kb OSCAR cDNAs described in Example 1, supra, were sequenced according to standard sequencing techniques. The cDNA sequence from each of these clones is set forth in FIG. 1A (1.8 kb OSCAR cDNA), and FIG. 1B (1.0 kb OSCAR cDNA) and in SEQ ID NOS:1 and 2, respectively. A comparison of these two nucleic acid sequences reveals that the two clones differ only in the 3′-untranslated region. Each clone encodes the same predicted amino acid sequence, which is set forth in FIG. 1C (SEQ ID NO:3). Sequence analysis of the clone containing the above described 4.0 kb OSCAR cDNA revealed that this clone encodes the same polypeptide, but is derived from an unprocessed (i.e., unspliced) OSCAR mRNA that contains intron sequences from the OSCAR genomic sequence.

Sequence analysis of the clone OCL178 confirmed that the cDNA contained in this clone corresponds to a fragment of a full length OSCAR cDNA sequence. Specifically, the OSCAR nucleic acid sequence contained in OCL178 (FIG. 2A; SEQ ID NO:4) corresponds to a fragment of a full length murine OSCAR cDNA sequence that encodes amino acid residues 161-165 of the OSCAR polypeptide sequence set forth in FIG. 1C (SEQ ID NO:3). The amino acid sequence of this particular fragment is also set forth separately in FIG. 2B and in SEQ ID NO:5.

Amino acid sequence analysis of the predicted murine OSCAR polypeptide indicates that OSCAR is a novel Ig-like receptor of 264 amino acid residues. The full length murine OSCAR polypeptide contains a signal peptide sequence (corresponding to amino acid residues 1-16), two Ig-like domain sequences (corresponding to amino acid residues 17-122 and 123-228, respectively), a single transmembrane domain sequence (corresponding to amino acid residues 229-247) and a short cytoplasmic tail sequence (corresponding to amino acid residues 248-264). It is understood that the amino acid residue numbers used to delineate these individual domains are approximate.

A search using the BLAST family of algorithms for homologous sequences in the NCBI nucleic acid and protein databases confirmed that the OSCAR nucleic acid and polypeptide sequences set forth in FIGS. 1A-C (SEQ ID NOS:1-3) are novel. Neither nucleic acid nor protein sequences corresponding to the murine OSCAR sequences described here were identified in these databases. However, the OSCAR polypeptide sequence did show significant sequence homology to two other Ig-like receptors. Specifically, a search of the NCBI protein database using the BLASTP algorithm revealed that the murine OSCAR polypeptide (FIG. 1C; SEQ ID NO:3) has 26.4% identity to murine PirA (Accession No. AAC53217.1) and 24.2% identity to the protein bovine FcαR (Accession No. P24071).

The transmembrane domain of the OSCAR polypeptide sequence shows amino acid sequence similarity to other Ig-like receptors, including murine PirA and bovine FcαR as described supra. In addition, the presence of a conserved arginine in the transmembrane sequence of the OSCAR polypeptide (amino acid residue 231 in FIG. 1C and in SEQ ID NO:3) is indicative of an association activity with transmembrane signaling adapter motifs. Such signaling adapters can be readily identified, for example by identifying proteins which co-immunoprecipitate with an OSCAR polypeptide or with a fragment of an OSCAR polypeptide that preferably comprises all or part of the transmembrane sequence (see Screening Assays, supra).

Ig-like receptors are known to participate in the regulation of development and/or function of cells expressing these receptors. Further, the activity of Ig-like receptors is mediate through binding with specific ligands, usually at the Ig-like domain(s). Thus, the sequence analysis of the murine OSCAR polypeptide depicted in FIG. 1C and in SEQ ID NO:3 supports the finding that OSCAR interacts with an OSCAR specific ligand (referred to herein as OSCAR-L) and that such an interaction modulates the development and function of osteoclast cells.

### Example 3

### Murine and Human Genomic DNA Hybridizes to Murine OSCAR cDNA

The example discloses the identification of human genomic DNA which hybridizes to the murine OSCAR cDNA. The human OSCAR genomic DNA was further characterized through BLAST searches which are also described here.

### Materials and Methods

Southern blot analysis. Southern blot analysis was performed at 42° C. for 16 hours using low stringency hybridization buffer (30% formamide, 10 mM Tris, pH 7.6, 2.5× Denhardt's solution, 5×SSC, 0.5% SDS, 1.5 mg/ml sonicated salmon sperm DNA). The membrane was washed twice at 50° C. for 20 minutes per wash using a low stringency washing buffer (0.5×SSC, 1% SDS).

### Results and Discussion

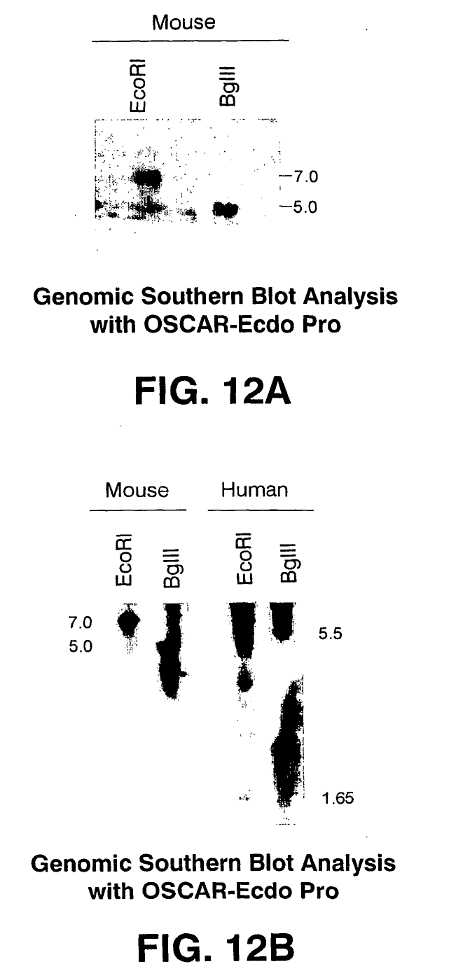

Murine OSCAR is derived from a single gene. Murine genomic DNA was digested with EcoRI or Bgl II restriction enzymes and analyzed by Southern Blot analysis with ³²P-labeled cDNA encoding the full length murine OSCAR polypeptide sequence set forth in FIG. 1C (SEQ ID NO:3). A 7.0 kb EcoRI fragment and 5.0 kb BglII fragment hybridized to the OSCAR probe (FIG. 12A). These results show that the murine OSCAR sequences identified in the 4.0 kb, 1.8 kb and 1.0 kb alternatively spliced cDNAs described supra are alternatively spliced transcripts of a single murine gene.

Humans genomic DNA hybridizes to murine OSCAR nucleic acids. Human genomic DNA was also digested with EcoRI and BglII restriction enzymes and analyzed by Southern Blot analysis using the same full length OSCAR cDNA probe and hybridization conditions that were used to analyze murine genomic DNA (supra). The murine OSCAR cDNA probe hybridizes with an approximately 1.65 kb EcoRI fragment, and with an approximately 5.5 kb BglII fragment of human genomic DNA (FIG. 12B). Thus, a human OSCAR homolog also exists which can be detected and identified by hybridization to murine OSCAR nucleic acid molecules of the present invention.

Identification and characterization of a human OSCAR gene. The BLASTN algorithm was used with its default parameters to search the NCBI nucleic acid databases and identify sequences homologous to the murine OSCAR cDNA sequences shown in FIGS. 1A-B (SEQ ID NOS:1-2). These databases contains, not only the nucleic acid sequences of numerous known human genes, but also contains partial human genomic sequences.

The BLAST search revealed that portions of the nucleotide sequence contained in the human chromosome 19 clone CTD-3093 (GenBank Accession No. AC012314.5; GI:771547) share homology to the murine OSCAR cDNA sequence. Thus, a human OSCAR gene is located on this chromosome. The exons of this genomic human OSCAR nucleic acid sequence were identified by comparing the human chromosome 19 sequence to the murine OSCAR cDNA sequence.

FIGS. 7A-D and SEQ ID NO:12 set forth the nucleotide sequence of the region on human chromosome 19 which contains the novel human OSCAR gene. In particular, the nucleotide sequence set forth in SEQ ID NO:12 and in FIGS. 7A-D corresponds to the sequence of nucleotides 117001-124920 from the sequence of human chromosome 19 clone CTD-3093 deposited in the GenBank database (Accession No. AC012314.5; GI:7711547). Exons of the novel OSCAR genomic sequence contained within this chromosomal region are indicated by upper case characters in FIGS. 7A-D, whereas the intron sequences within the OSCAR gene are set forth in lower case characters. The nucleotide residue numbers of the intron/exon boundaries of this novel OSCAR genomic sequence are also set forth in TABLE 1, supra, with respect to the nucleotide residue numbers in SEQ ID NO:12.

To further characterize the human OSCAR gene, a cDNA library derived from human osteoclast cells was screened using techniques similar to those described, supra, for screening a murine cDNA library. Three splice variants, or isoforms, of human OSCAR were identified. These three isoforms are referred to herein as the C18 human OSCAR isoform, the C16 human OSCAR isoform, and the C10 human OSCAR isoform, respectively. cDNA, sequences for each of these three isoforms are set forth in FIG. 3A and SEQ ID NO:6 (for the C18 human OSCAR isoform), in FIG. 4A and SEQ ID NO:8 (for the C16 human OSCAR isoform) and in FIG. 5A and SEQ ID NO:8 (for the C10 human OSCAR isoform). Predicted amino acid sequences for OSCAR polypeptides encoded by each of these three isoforms are also provided herein in FIG. 3B and SEQ ID NO:7 (for the C18 human OSCAR isoform), in FIG. 4B and SEQ ID NO:9 (for the C16 human OSCAR isoform) and in FIG. 5B and SEQ ID NO:11 (for the C10 human OSCAR isoform). The sequences were later resequenced and confirmed with only minor sequencing corrections. In particular, nucleic acid residue 328 of the human OSCAR C18 isoform's cDNA (shown in FIG. 3A and in SEQ ID NO:6) was determined to be a guanine (G) rather than a thymine as originally sequenced. This correction leads to a minor change in the predicted amino acid sequence (shown in FIG. 3B and in SEQ ID NO:7) for the C18 splice variant, in which amino acid residue 97 is a serine (S or Ser), rather than an isoleucine (I or Ile) as originally predicted. The corrected nucleic acid and amino acid sequences for the C18 isoform are presented here in FIGS. 3A and 3B, and in SEQ ID NOS:6 and 7, respectively.

Similarly, nucleic acid residue 295 of the human OSCAR C10 isoform cDNA (shown in FIG. 5A and in SEQ ID NO:10) was determined to be a guanine (G) rather than a thymine as originally sequenced. This correction leads to a minor change in the predicted amino acid sequene (shown in FIG. 5B and in SEQ ID NO:11) for the C10 splice variant, in which amino acid residue 86 is a serine (S or Ser) rather than an isoleucine (I or Ile) as originally predicted. The corrected nucleic acid and amino acid sequences for the C10 isoform are presented here in FIGS. 5A and 5B, and in SEQ ID NOS:10 and 11, respectively.

An alignment of the human and murine OSCAR polypeptide sequences (FIG. 6) confirms that these sequences share a very high level of homology. In particular, the two sequences were found to be 74.6% (i.e., about 75%) identical.

### Example 4

### Fusion Proteins Containing Extracellular Domains of OSCAR Modulate Maturation and Activity of Osteoclast Cells

This example describes particular fusion polypeptides that comprise OSCAR amino acid sequences of the invention. The example also describes a preliminary experiment demonstrating that such fusion polypeptides are capable of binding an OSCAR specific ligand, and can be used to modulate osteoclast cell activity.

### Materials and Methods

FACS Analysis. FACS analyses were performed according to routine methods described, e.g., by Sharrow, Chapters 5.1-5.2 in Current Protocols in Immunology, Vol. I (Coligan et al., eds.) John Wiley & Sons, Inc; and by Kevin et al., Chapter 5.3 in Current Protocols in Immunology, Vol. I (Coligan et al., eds.) John Wiley & Sons, Inc.

Generation of fusion proteins. Fusion proteins containing the extracellular domain of OSCAR were generated as described below. PCR was used to amplify the relevant OSCAR domains and the human IgG1 Fc portion using Herculase (STRATAGENE).

Generation of OSCAR-Fc in pcDNA. A nucleic acid sequence encoding the extracellular domain of the murine OSCAR polypeptide set forth in FIG. 1C (SEQ ID NO:3; amino acid residues 1-228) was PCR amplified from an OSCAR cDNA plasmid using primers referred to as 5′OSCAR-Met-RI and 3′-OSCAR-Ec-Bgl ii (SEQ ID NOS:13-14, respectively). The PCR product was digested with EcoRI and BglII.

The Fc region of human IgG1 was PCR amplified from a human cDNA plasmid using primers referred to as 5′-Human IgG1 (SEQ ID NO:15) and 3′-Human IgG1 (SEQ ID NO:16). The product from this second PCR reaction was digested with Bgl II and XbaI. The digested products from both PCR reactions were then ligated into the pcDNA1 expression vector using EcoRI and XbaI.

The nucleic acid sequences of the primers used are as follows:

| | | |
|---|---|---|
| 5′-OSCAR-Met-RI: | 5′-GGAATTCACCATGGTCCTGTCGCTGATACTC-3′ | (SEQ ID NO:13) |
| 3′-OSCAR-Ec-Bgl ii: | 5′-GAAGATCTGTTTCCCTGGGTATAGTCCAA-3′ | (SEQ ID NO:14) |
| 5′-Human IgG1: | 5′-GAGCCGCTCGAGGAATTCGTCGACAGATCTTGTGACAAAACTCAC-3′ | (SEQ ID NO:15) |
| 3′-Human IgG1: | 5′-GGCCGCTCTAGAACTAGTTCATTT-3′ | (SEQ ID NO:16) |

Generation of OSCAR-Fc in pMT/V5-His. OSCAR-Fc cDNA was ligated into the Drosophila expression vector, pMT/V5-His (Invitrogen) using EcoRI and XbaI.

Generation of GST-OSCAR in pGEX6p-1. A nucleic acid sequence encoding the extracellular domain of the OSCAR polypeptide set forth in FIG. 1C (SEQ ID NO:3; amino acid residues 1-228) was PCR amplified from an OSCAR cDNA plasmid using primers referred to as 5′-OSCAR-Ec-HR (SEQ ID NO:17) and 3′-OSCAR-Ec-STOP-XhoI (SEQ ID NO:18). The PCR product was digested with EcoRI and XhoI, and ligated into a pGEX6p-1 vector using EcoRI and XhoI. The vector was transfected and expressed in E. coli BL21 strain cells using IPTG and X-gal induction methods (see, e.g., Sambrook et al., 1989, supra).

The nucleic acid sequences of the primers used are as follows:

| | | |
|---|---|---|
| 5′-OSCAR-Ec-HR: | 5′-CCCAAGCTTGAATTCGACTTCACACCAACAGCG-3′ | (SEQ ID NO:17) |
| 3′-OSCAR-Ec- | 5′-CCGCTCGAGTCAGTTTCCCTGGGTATAGTCCAA-3′ | (SEQ ID NO:18) |
| STOP-Xho I: | | |

Generation of GST-F-OSCAR in pGEX6p-4. A nucleic acid sequence encoding the first Ig-like domain (i.e., amino acid residues 17-122) of the OSCAR polypeptide set forth in FIG. 1C (SEQ ID NO:3) was PCR amplified from an OSCAR cDNA plasmid using primers referred to as 5′-OSCAR-Ec-HR (SEQ ID NO:17, described supra) and 3′-OSCAR-EcI-STOP-XhoI (SEQ ID NO:19). The PCR product was digested with EcoRI and XhoI, and ligated into a pGEX6p-1 vector. The vector was transfected and expressed in E. coli BL21 strain cells IPTG and Xgal induction (see, e.g., Sambrook et al., 1989,supra).

The nucleic acid sequences of the primers used are as follows:

| | | |
|---|---|---|
| 5′-OSCAR-Ec-HR: | 5′-CCCAAGCTTGAATTCGACTTCACACCAACAGCG-3′ | (SEQ ID NO:17) |
| 3′-OSCAR-EcI- | 5′-CCGCTCGAGTCAATCCGTTACCAGCAGTTC-3′ | (SEQ ID NO:19) |
| STOP-XhoI: | | |

Generation of GST-S-OSCAR in pGEX6p-1. A nucleic acid sequence encoding the second Ig-like domain (i.e., amino acid residues 123-228) of the OSCAR polypeptide set forth in FIG. 1C (SEQ ID NO:3) was PCR amplified from an OSCAR cDNA plasmid using primers referred to as 5′-OSCAR-EcII-HR (SEQ ID NO:20) and 3′-OSCAR-Ec-STOP-XhoI (SEQ ID NO:18, described supra). The PCR product was digested with EcoRI and XhoI, and ligated into a pGEX6p-1 vector. The vector was transfected and expressed in E. coli BL21 strain cells IPTG and Xgal induction (see, e.g., Sambrook et al., 1989, supra).

The nucleic acid sequences of the primers used are as follows:

| | | |
|---|---|---|
| 5′-OSCAR-EcIII-RI: | 5′-GGAATTCGATCAGCTCCCCAGACCAT-3′ | (SEQ ID NO:20) |
| 3′-OSCAR-Ec- | 5′-CCGCTCGAGTCAGTTTCCCTGGGTATAGTCCAA-3′ | (SEQ ID NO:18) |
| STOP-Xho I: | | |

Purifcation of OSCAR-Fc. OSCAR-IgG was purified from the culture supernatant using Protein A chromatography as described (Sambrook et al., 1989, supra).

Osteoclast maturation assay. Osteoblast cells were isolated from calvariae of wild type and TRANCE knockout mice as described by Suda et al. (Methods in Enzymolozy 1977, 282: 223-35). In a co-culture experiment of osteoblast cells and hemopoietic precursors, bone marrow cells (1×10⁵ cells) and osteoblast cells (1×10⁴ cells) were co-cultured in α-MEM containing 10% FBS in the presence of 1×10⁻⁷ M or 1×10⁻⁸ M 1α,25(OH)₂D₃ in 96 well plates (0.2 m/well). 20 μg/ml of OSCAR-IgG or human IgG1 was added to the cultures to observe the role of OSCAR during the differentiation of osteoclast cells. Cultures were fed every 3 days by replacing 160 μl of old medium with fresh medium. After culturing for 6 or 7 days, cells were fixed and stained for TRAP (SIGMA) as described (Wani et al., Endocrinology 1999, 140:1927-1935). The number of TRAP (+) multinucleated osteoclast cells with more than three nuclei were counted from each of the wells.

### Results and Discussion

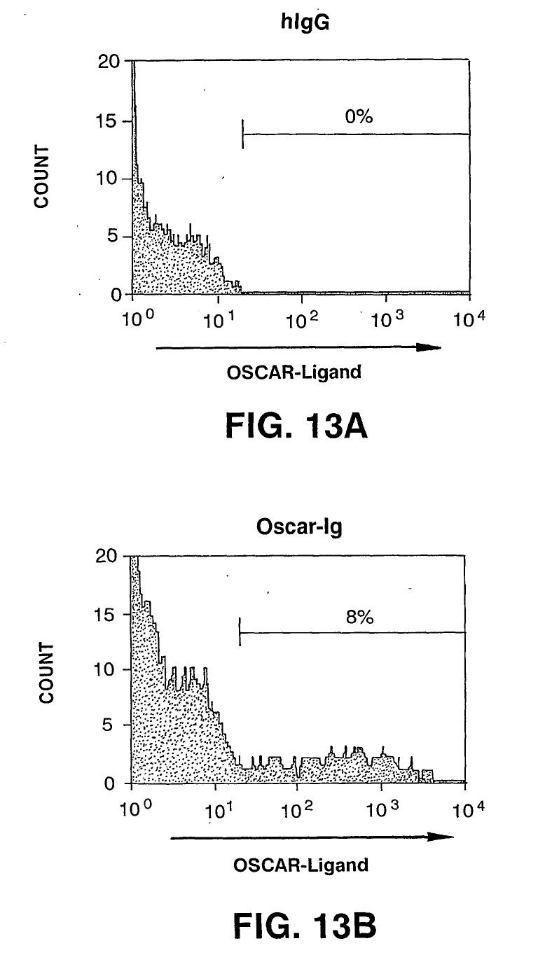

OSCAR-L is expressed on the surface of osteoblasts. Primary osteoblast cells derived from murine calvaria were stained (i.e., incubated) with either an isotype-control human IgG1 protein (FIG. 13A) or the OSCAR-Ig fusion polypeptide described in the Materials and Methods section, supra (FIG. 13B), followed by incubation with a PE-conjugated anti-human IgG1 antibody. The cells were then analyzed by FACS to detect the levels of PE-fluorescence associated with these cells. The results are shown in the histograms set forth in FIGS. 13A-B. Specifically, these histograms indicate, for each experiment, the number of cells (vertical axis) observed having a particular level of PE-fluorescence (horizontal axis). Cells having higher levels of observed fluorescence are indicative of higher amounts of PE-conjugated antibody binding to those cells. The PE-conjugated anti-human IgG antibody, in turn, indirectly binds to cells by binding either hIgG (FIG. 13A) or OSCAR-Ig (FIG. 13B) bound-to the cell surface, e.g., by binding to an OSCAR specific ligand.

PE fluorescence levels increased significantly when the cells were incubated with the OSCAR-Ig fusion polypeptides relative to PE fluorescence on cells that were incubated with IgG1 controls. Thus, the data demonstrate that osteoblast cells express a compound (i.e., an OSCAR ligand) which specifically binds to an OSCAR polypeptide of the invention. In particular, because the IgG1 polypeptide did not bind to the osteoblast cells, the OSCAR ligand expressed by those cells specifically binds to OSCAR polypeptide sequences of the OSCAR-Ig fusion polypeptide used in these experiments, and does not bind to the IgG1 sequence of that fusion polypeptide.

PE fluorescence levels were not significantly altered in identical experiments where osteoblast cells were also treated with either vitamin D₃ or parathyroid hormone, which are known to increase osteoblast and osteoclast cell activity, respectively. Thus, expression of the OSCAR ligand is not affected by such compounds.

The addition of OSCAR-Ig modulates osteoclast cell activity. A pilot experiment was performed to test the ability of OSCAR polypeptides to modulate osteoclast and/or osteoblast cell activity. Murine bone marrow and osteoblast cells were co-cultured as described above in the osteoclast maturaturation assay. Observation of the co-cultures at a single, designated time point did not reveal the presence of mature (i e., multinucleated) osteoclast cells in TRAP stained co-cultures that were treated with an isotype-control human IgG1 protein. Further treatment of co-cultured bone marrow and osteoblast cells with vitamin D₃ (100 nM) and the control IgG1 protein induced formation of multi-nucleated osteoclast cells, as detected by TRAP staining. Treatment of co-cultured bone marrow and osteoblast cells with lower levels of vitamin D₃ (10 nM), resulted in the formation of some osteoclast precursor cells, but no mature, multinucleated osteoclast cells were detected by TRAP staining. These results are expected, due to the known ability of vitamin D₃ to activated TRANCE in osteoblast cells and thereby induce osteoclast cell maturation. Indeed, in control experiments where TRANCE knock-out osteoblast cells were co-cultured with bone marrow cells, treatment with similar levels of vitamin D₃ had no effect on osteoclast cell maturation.

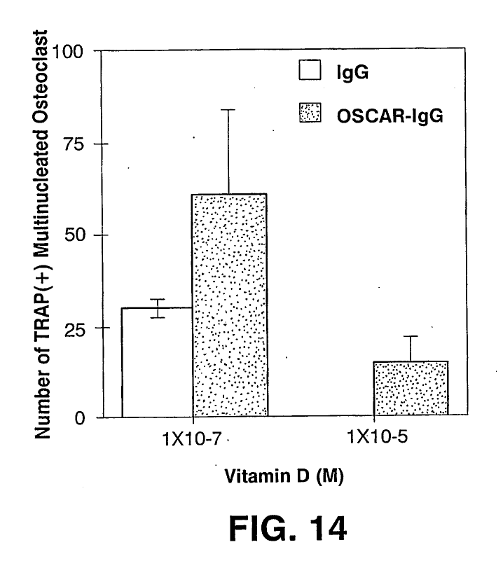

By contrast, treatment of the co-cultured cells with vitamin D₃ (10 or 100 nM) and the OSCAR-Ig fusion polypeptide described supra, resulted in an apparent increase in the numbers of mature, multinucleated osteoclast cells formed relative to the above-described control experiments. These results are shown, quantitatively, in FIG. 14.

The observation of TRAP (+) multinucleated cells is indicative of osteoclast cell maturation. Increased numbers of these cells in the presence of the OSCAR fusion polypeptide therefore suggests that osteoclast cell activity has been modulated in some way under the specific conditions in this particular pilot experiment. Without being limited to any particular theory or mechanism of action, the soluble OSCAR polypeptide used in these experiments is thought to competitively bind to the OSCAR-specific ligand expressed by the osteoblast cells, thereby preventing interaction between the OSCAR-specific ligand and OSCAR polypeptides expressed by the bone marrow cells (e.g., by osteoclast precursor cells and immature osteoclast cells in the bone marrow cells).

The data-presented in these experiments therefore indicate that the OSCAR polypeptides and OSCAR specific ligands of the present invention may be used to modulate the maturation and/or activity of osteoclast cells, thereby enabling the modulation of processes associated with the growth, development, repair, degradation, resorption or homeostasis of bone tissue.

### Example 5

### Fusion Proteins Containing Extracellular Domains of OSCAR Inhibit Maturation and Activity of Osteoclast Cells

This example describes additional, more definitive experiments that were performed after preliminary data (presented in Example 4, supra) indicated that OSCAR may modulate osteoclast cell activity. In particular, data from kinetic measurement of osteoclast cell maturation are presented. These data further characterize the OSCAR fusion polypeptide's ability to modulate osteoclast cell activity.

### Materials and Methods

Generation and purification of OSCAR-Fc fusion proteins. Preparation of an OSCAR-Ig fusion protein was accomplished as described above in Example 4.

Kinetic measurement of osteoclast maturation. Bone marrow cells and osteoblast cells were isolated from wild-type and TRANCE knock-out mice and co-cultured in 96-well plates as described in Example 4, supra. Floater cell cultures were also prepared that contained a higher population of osteoclast specific precursor cells than the ordinary co-cultures. Briefly, the floater cultures were prepared by treating total bone marrow cultures (3×10⁵ cells) with 5 ng/ml of macrophage-colony stimulating factor (M-CSF), followed by elimination of the resulting macrophage cells.

10 nM vitamin D₃, was added to the cultures to stimulate osteoclast cell maturation. 20 μg/ml of either OSCAR-Ig or a human IgG1 was also added to cultures to observe the role of OSCAR during osteoclast cell differentiation. Control cultures were also prepared that received either 10 nM vitamin D₃ alone (i.e. no OSCAR or IgG1), or were cultured in medium without adding vitamin D₃ or protein. After culturing for at least six days, the number of TRAP (+) multinucleated cells in a well was counted daily, as described supra, in Example 4.

Dentine resorption assay. A dentine resorption assay, which is indicative of bone resorption activity, was performed as previously described. See, for example, Tamura et al., J. Bone Miner. Res. 1993, 8(8): 953-60; and Suda et al., Methods in Enzymology 1997,282:223-25.

Briefly, co-cultures of mouse osteoblast and bone marrow cells were prepared as described above on dentine slices and in the presence of 10 nM vitamin D₃. 20 μg/ml of OSCAR-IgG or human IgG1 was added to the cultures to observe the role of OSCAR on ostseoclast cell activity (i.e., bone or dentine resorption). Control cultures were also grown on dentine slices in the presence of either 10 nM vitamin D₃ alone (i.e., no OSCAR-Ig or IgG1), or without exposure to either vitamin D₃ or fusion protein.

After culturing on dentine slices for 6 days, the cells were stained for TRAP to detect multinucleated osteoclast cells. Resorption pits in the dentine slices were visualized by light microscopy.

### Results and Discussion

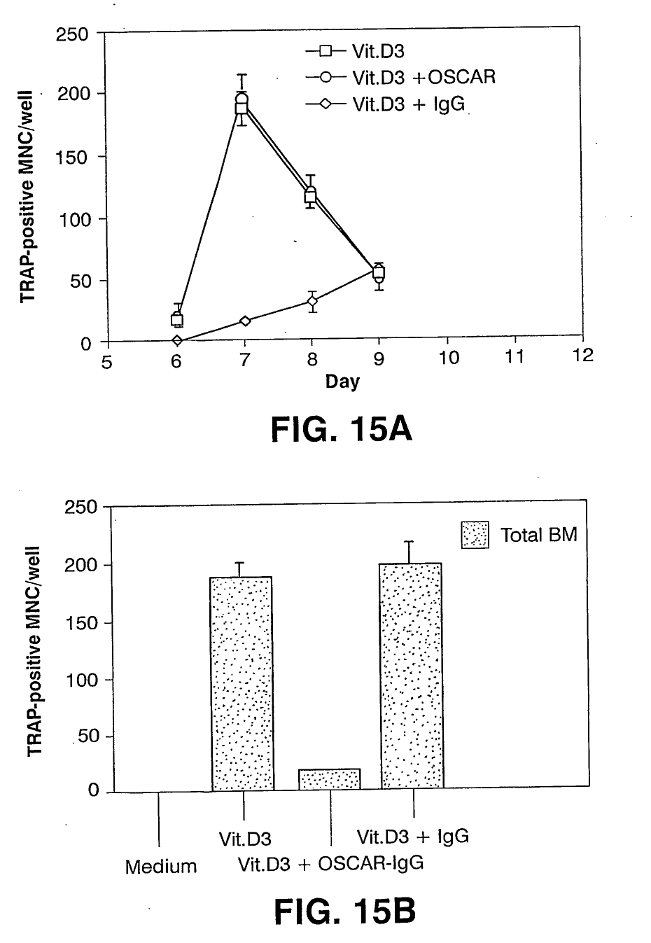

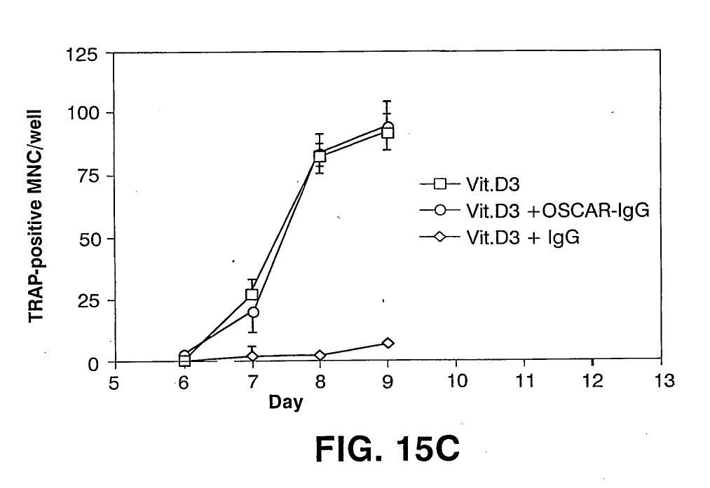

Addition of OSCAR-Ig decreases tile number of TRAP (+) multinucleated cells. To better characterize how OSCAR may modulate osteoclast cell maturation and/or activity, kinetic experiments were performed that monitored osteoclast cell maturation both in the presence and in the absence of an OSCAR polypeptide, and over a period of several days. Kinetic experiments are necessary to fully characterize the effect OSCAR may have on osteoclast cells, since mature osteoclast cells do not normally remain viable in culture. Thus, a factor that stimulates osteoclast cells may be characterized by an initial increase in the number of mature (e.g., multinucleated) osteoclast cells observed in culture, followed by lower numbers due to post-maturation cell death. FIGS. 15A-15C show data obtained for kinetic experiments that used co-cultured murine bone marrow and osteoblast cells (FIGS. 15A-15B), and floater cells cultures (FIG. 15C) that contain a higher population of osteoclast-specific precursor cells.

As shown quantiatively in FIG. 15A, vitamin D₃-stimulated osteoclast maturation in total bone marrow cultures, indicated by the number of multi-nucleated TRAP (+) cells, peaks dramatically about 7 days after treatment. This initial increase is followed, however, by rapid, incremental decreases in activity by days 8 and 9, respectively. In contrast, treatment of co-cultures with vitamin D₃ and the OSCAR-IG fusion polypeptide resulted in a significant decrease in the number of TRAP (+) cells formed on days 6 through 9 relative to the control experiments.

A bar graph indicating the number of mature (i.e., TRAP (+), multi-nucleated) cells present in the co-cultures 7 days after treatment, when stimulated osteoclast cell maturation had peaked, is shown in FIG. 15B. Cells cultured in the presence of either vitamin D₃ alone or vitamin D₃ with a control IgG protein show markedly elevated numbers of mature osteoclast cells (between about 150-200 cells per well). The number of mature osteoclast cells is severely reduced (i e., fewer the 50 cells per well) in co-cultures with vitamin D₃ and the OSCAR-Ig fusion protein.

The kinetic curve for floater cells cultures (FIG. 15C) shows a similar, but more gradual increase in the number of TRAP (+) cells induced by vitamin D₃ about 7 days after treatment and continuing to at least day 9. Treatment of the floater cell cultures with vitamin D₃ and a control human IgG1 protein results in a similar growth curve, as expected. However, treatment of the floater cell cultures with vitamin D3 and the OSCAR-Ig fusion protein significantly inhibits osteoclast cell maturation in a manner similar to the inhibition observed for the co-cultured bone marrow and osteoblast cell cultures shown in FIG. 15A.

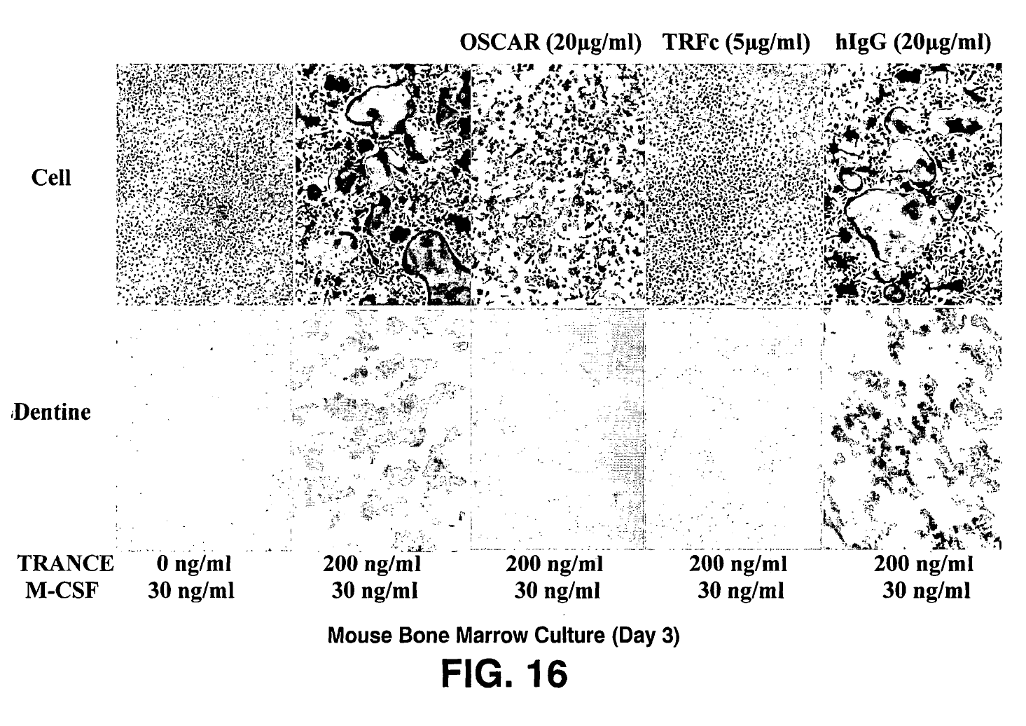

OSCAR-Ig inhibits dentine resorption by osteoclast cells. Dentine resorption assay experiments were also performed as previously described (see, e.g., Yasuda et al., Proc. Natl. Acad. Sci. U.S.A. 1998, 95:3597-3602; and Tamura et al., J. Bone Miner. Res. 1993, 8:953-960) to more thoroughly characterize the effect of OSCAR on osteoclast and/or osteoblast cell activity. More specifically, the assay detects the effect of OSCAR on bone or dentine resorption. Panels A-E in FIG. 16 show photomicrographs of TRAP (+) stained murine osteoblast and bone marrow cells co-cultured on dentine slices. Panels F-J in FIG. 16 show photomicrographs of the corresponding dentine slices. Dark stains in the micrographs indicate pits in the slices where dentine has been resorbed.

As expected, cells co-cultured on dentine slices without vitamin D₃ (FIG. 16A) exhibit little or no osteoclast cell maturation, indicated by the lack of TRAP (+) cells. Similarly, no resorption is indicated on the corresponding dentine slices (FIG. 16F). By contrast, co-cultures on dentine slices exhibit markedly increased TRAP (+) staining when exposed to either 10⁻⁸ M vitamin D₃ alone (FIG. 16B), or 10⁻⁸ M vitamin D3 with a control IgG protein (20 μg/ml) (FIG. 16E). Dark stains indicating dentine resorption are also observed on the corresponding dentine slices (FIGS. 16G and 16J, respectively). These resorption pits correlate with the TRAP (+) stained areas in the corresponding cell cultures, confirming as expected that increased osteoclast cell maturation correlates with increased resorption.

Contrary to what was observed with these positive controls, co-cultures on dentine that were incubated with OSCAR-Ig (20 μg/mL) along with 10⁻⁸ M vitamin D₃ exhibit very little or no TRAP (+) staining (FIG. 16C), and there is little or no dentine resorption (FIG. 16H). Thus, treatment with OSCAR-Ig actually inhibits osteoclast cell activity and, more specifically, inhibits bone or dentine resorption.

Negative control experiments were also performed to verify the results obtained with OSCAR-Ig. Specifically, co-cultures of osteoblast and bone marrow cells were incubated with 10⁻⁸ M vitamin D₃ and murine TRANCE inhibitor (mTR-Fc), a known inhibitor of osteoclast cell activity (Fuller et al., J. Exp. Med. 1998, 188:997-1000). As expected, little or no TRAP (+) staining was seen in those co-cultures (FIG. 16D), and very little, if any, dentine resorption occurred (FIG. 16I).

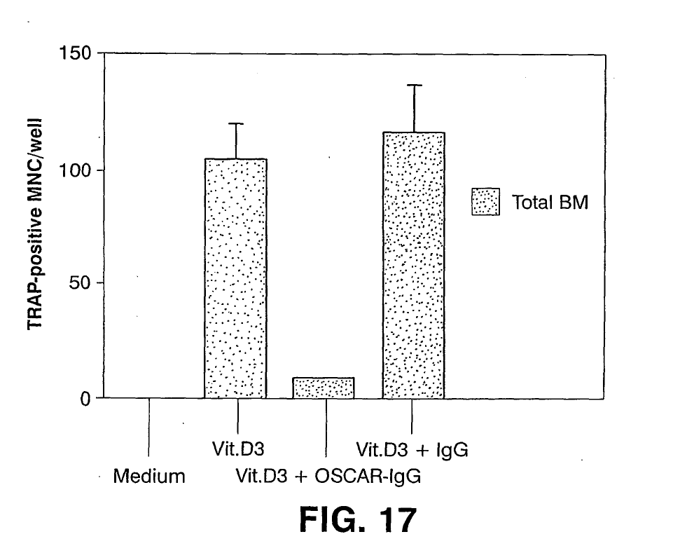

The results from these dentine resorption experiments are shown quantitatively in FIG. 17. Specifically, the bar graph in this figure shows the average number of dentine resorption pits counted on each slice of co-cultured osteoblast and bone marrow cells. Over 100 pits were observed, on average, on slices incubated with vitamin D₃, either alone (102.7±16.8) or with the control IgG1 protein (114.7±22.2). By contrast, incubation with OSCAR-Ig inhibits resorption by more than a factor of 10, with fewer than 10 pits observed on each of those slices (7±2).

The data from these experiments therefore confirm that OSCAR polypeptides and OSCAR-specific ligands of the present invention may be used to modulate the maturation and/or activity of osteoclast cells, including activities such as bone or dentine resorption that may be measured or estimated, e.g., by the dentine resorption assay described here. In particular, and without being limited to any particular theory or mechanism of action, the soluble OSCAR polypeptide used in these experiments is thought to competitively bind to the OSCAR-specific ligand expressed by osteoblast cells, thereby preventing OSCAR polypeptides expressed by the bone marrow cells (e.g., by osteoclast precursor cells, and by immature osteoclast cells in the bone marrow cells) from being activated. As a result, osteoclast maturation and activity, which is normally activated or stimulated by the binding of OSCAR to its specific ligand, is inhibited. Using the methods and compositions of this invention, therefore, processes that are associated with osteoclast cell activity can be readily modulated, including but not limited to processes associated with the growth, development, repair, degradation, resorption or homeostasis of bone tissue.

### Example 6

### The Ability of OSCAR-Ig FUSION PROTEINS to Inhibit Osteoclast Maturation is Cross-Reactive Among Species

Examples 4 and 5 above, describe the preparation and isolation of a soluble OSCAR polypeptide (referred to as OSCAR-Ig or mOSCAR-Ig) using OSCAR nucleic acid and amino acid sequences from mouse. Those examples also demonstrate the use of that soluble OSCAR polypeptide to modulate the maturation and activity of murine cells.

The present example describes the preparation and isolation of a soluble OSCAR polypeptide (referred to as hOSCAR-Ig) using OSCAR nucleic acid and amino acid sequences derived from human and, further, demonstrates the use of this soluble human OSCAR polypeptide to modulate the maturation and activity of human cells. Data is also presented showing that OSCAR is cross-reactive among different species. In particular, the present Example demonstrates the use of a soluble murine OSCAR polypeptide to modulate the maturation and activity of human cells. Similarly, use of a human OSCAR polypeptide to modulate maturation and activity of murine cells is also described.

### Materials and Methods

Generation of hOSCAR-Fc in pcDNA. A nucleic acid sequence encoding the extracellular domain of the human OSCAR polypeptide set forth in FIG. 3A (SEQ ID NO:6; amino acid residues 1-219) was PCR amplified from a hOSCAR cDNA plasmid using primers referred to as 5′hOSCAR-Met-XhoI and 3′-hOSCAR-Ec-HindIII (SEQ ID NOS.21-22, respectively). The PCR product was digested with XhoI and HindIII.

A thrombin site was inserted at the end the human OSCAR by further amplifying the product generated above using primers referred to as Thrombin-S and Thrombin-AS (SEQ ID NOS: 23-24, respectively).

The Fc region of human IgG1 was PCR amplified from a human cDNA plasmid using primers referred to as 5′-Human IgG1 (SEQ ID NO:15) and 3′-Human IgG1 (SEQ ID NO:16). The product from this third PCR reaction was digested with Bgl II and XbaI. The digested products from both PCR reactions were then ligated into the pcDNA1 expression vector using Ex6 and XbaI.

The nucleic acid sequences of the primers used are as follows:

| | | |
|---|---|---|
| 5′-hOSCAR-Met-XhoI: | 5′-CCGCTCGAGACCATGGCCCTGGTGCTGAT-3′ | (SEQ ID NO:21) |
| 3′-hOSCAR-Ec-HindIII: | 5′-CCCAAGCTTTGATCCTCCTCCGTCTTCCCAGCTGATGACCA-3′ | (SEQ ID NO:22) |
| Thrombin-S: | 5′-CCCAAGCTTCTGGTTCCGCGTGGATCCGCG-3′ | (SEQ ID NO:23) |
| Thrombin-AS: | 5′-CGCGGATCCACGCGGAACCAGAAGCTTGGG-3′ | (SEQ ID NO:24) |
| 5′-Human IgG1: | 5′-GAGCCGCTCGAGGAATTCGTCGACAGATCTTGTGACAAAACTCAC-3′ | (SEQ ID NO:15) |
| 3′-Human IgG1: | 5′-GGCCGCTCTAGAACTAGTTCATTT-3′ | (SEQ ID NO:16) |

Generation of hOSCAR-Fc in pMT/V5-His. hOSCAR-Fc cDNA was ligated into the Drosophila expression vector, pMT/V5-His (Invitrogen) using XhoI and XbaI.

Purifcation of hOSCAR-Fc. hOSCAR-IgG was purified from the culture supernatant using Protein A chromatography as described (Sambrook et al., 1989, supra).

Generation of Human monocyte cultures. Blood leukocytes were collected by continuous filtration leukapheresis (CFL) using a Leukopak filter and then, subjected to counterflow centrifugal elutriation to yield distinct fractions separated by mass. The fraction containing about 90% purity for CD14+ cells are monocytes. The monocytes were maintained and induced to differentiate into human osteoclasts as described in Matsuzaki et al., Biochem. Biophys. Res. Comm. 1998, 246(1):199-204.

Murine bone marrow cell cultures. Co-cultures of murine osteoblast and bone marrow cells were prepared as described in Example 4.

Dentine resorption assay. A dentine resorption assay was performed according to routine protocols (see, Example 5, supra, and Tamura et al., J. Bone Miner. Res. 1993, 8(8):953-960) using human monocyte cell cultures that were prepared as described above.

### Results and Discussion

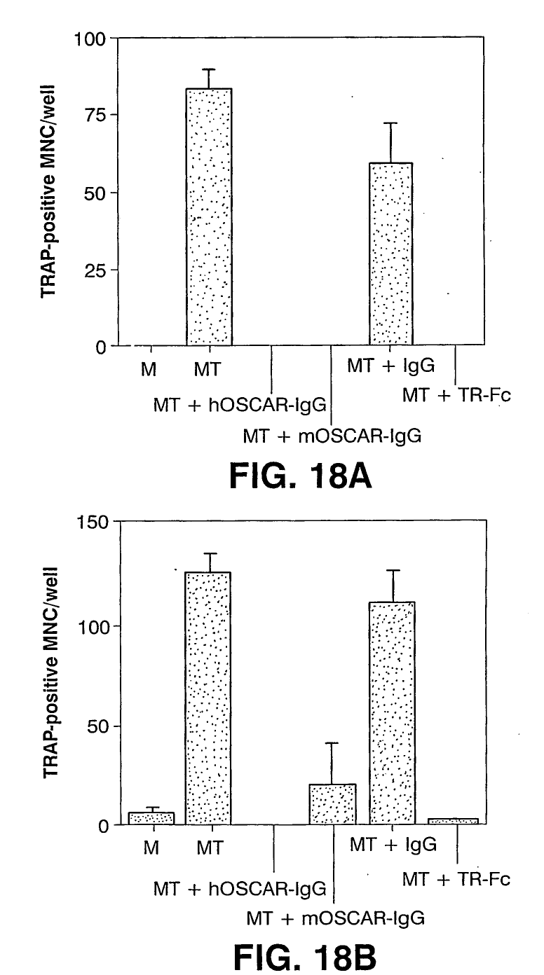

OSCAR-Ig inhibits maturation and activity of human osteoclast cells. Experiments that are similar to the experiments described in Examples 4 and 5, supra, were performed using soluble murine and human OSCAR polypeptides (mOSCAR-Ig and hOSCAR-Ig, respectively) to characterize the ability of OSCAR polypeptides to modulate the maturation and/or activity of human cells. Specifically, human monocyte cells were cultured in the presence of M-CSF (30 ng/ml), TRANCE (200 ng/ml) and 20 ng/ml of either soluble hOSCAR-Ig or mOSCAR-Ig, and TRAP (+) multi-nucleated cells were counted 5 and 10 days after exposure. These data are presented graphically in FIG. 18A (5 days post-exposure) and FIG. 18B (10 days post-exposure), respectively. Control experiments were also conducted where human monocytes were cultured with either M-CSF and TRANCE alone (i.e., without OSCAR-Ig), or with M-CSF, TRANCE and a human IgG1 polypeptide. For negative controls, human monocyte cells were cultured with M-CSF along (i.e., no TRANCE or OSCAR-Ig), and with M-CSF, TRANCE and the known osteoclast cell inhibitor TR-Fc (see, Example 5, supra).

As expected, very few or no TRAP (+) multi-nuclear cells were observed in cell cultures incubated with M-CSF alone (M) or with M-CSF, TRANCE and TR-Fc (MT+TR-Fc). See, lanes 1, 5 and 6, respectively, in FIGS. 18A and 18B. By contrast, incubation of human monocyte cells with either M-CSF and TRANCE alone (MT; lane 2 in FIGS. 18A and 18B), or with M-CSF, TRANCE and Ig (MT+IgG; lane 5 in FIGS. 18A and 18B) However, incubating the monocytes with hOSCAR-IgG (lane 3 in FIGS. 18A and 18B) inhibited those elevated osteoclast maturation levels. Incubation with mOSCAR-IgG (lane 4 in FIGS. 18A and 18B) had a similar effect. Somewhat more TRAP (+) multi-nucleated cells were seen after 10 days of incubation with mOSCAR-Ig compared to hOSCAR-Ig (FIG. 18B, lanes 4 and 3, respectively). Nevertheless, the number of TRAP (+) multi-nuclear cells seen after 10 days incubation with mOSCAR-Ig is more than an order of magnitude lower than the number seen when the human cells were incubated with M-CSF and TRANCE alone, or with IgG1. Thus, both human and murine OSCAR polypeptides are able to effectively modulate the maturation and activity of human osteoclast cells.

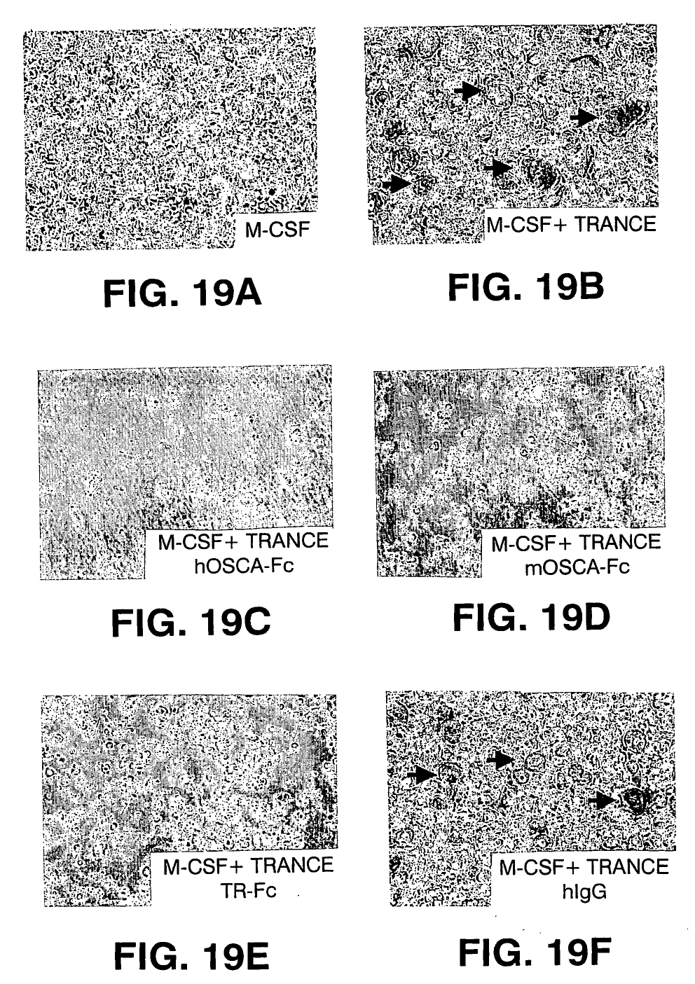

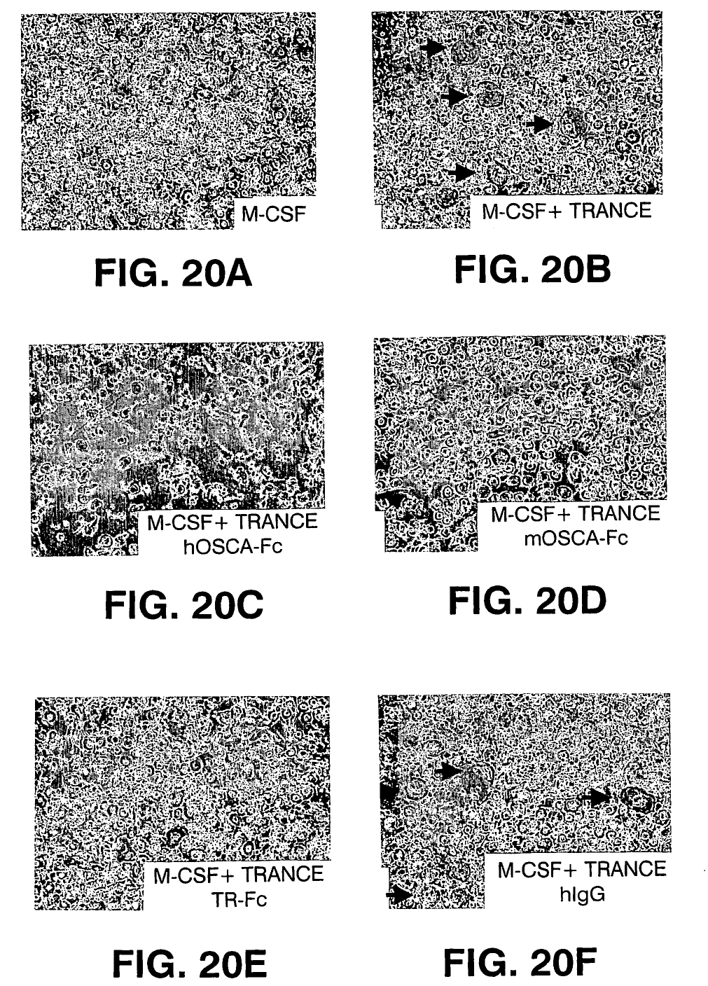

Photomicrographs from these cell cultures are shown in FIG. 19 (5 days post-exposure) and FIG. 20 (10 days post-exposure). Cultures that were incubated with M-CSF and TRANCE (FIGS. 19B and 20B) or with M-CSF, TRANCE and IgG1 (FIGS. 19F and 20F) had more multi-nuclear cells (indicated by arrows), whereas very few or no multi-nuclear cells can be seen in photomicrographs from cultures incubated with either hOSCAR-Ig (FIGS. 19C and 20C) or mOSCAR-Ig (FIGS. 19D and 20D).

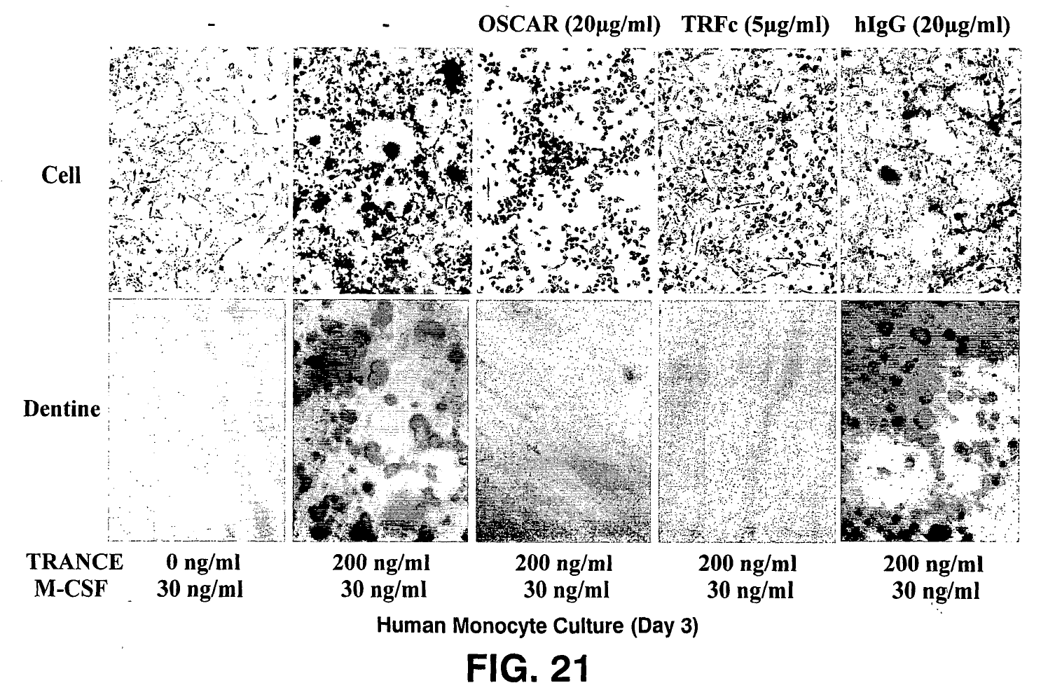

A dentine resorption assay (described in Example 5, supra) was also performed using human monocyte cell cultures to confirm the murine OSCAR polypeptide's ability to modulate human osteoclast cell activity. The results of these experiments are shown in FIGS. 21A-J. Specifically, panels A-E in FIG. 21 show photomicrographs of human monocyte cells cultured on dentine slices in the presence of 30 ng/ml M-CSF (FIG. 21A), 30 ng/ml M-CSF and 200 ng/ml TRANCE (FIG. 21B), M-CSF (30 ng/ml), TRANCE (200 ng/ml) and 20 μg/ml mOSCAR-Ig (FIG. 21C), M-CSF (30 ng/ml), TRANCE (200 ng/ml) and 5 μg/ml TR-Fc (FIG. 21D) and M-CSF (30 ng/ml), TRANCE (200 ng/ml) and 20 μg/ml hIgG1 (FIG. 21E). FIGS. 21F-J show photomicrographs of the dentine slices after the cell cultures in FIGS. 21A-E, respectively, have been washed away. Dark stains in these micrographs indicate pits where dentine has been resorbed.

Similar to what was observed in dentine resorption experiments that used murine cells (see, Example 5, supra, and FIGS. 17A-17J), very little or no evidence of dentine resorption was seen when human monocytes were cultured either with M-CSF alone (FIG. 21F) or with TR-Fc (FIG. 21I). However, significant resorption was observed when the human monocyte cells were cultured with TRANCE, either alone (FIG. 21G) or with a control IgG1 polypeptide (FIG. 21J). The elevated resorption levels observed in the presence of TRANCE were inhibited, however, when the human monocyte cells were incubated with mOSCAR-Ig (FIG. 21H).

The results from these experiments therefore demonstrate the both the maturation and activity of human cells (i.e., human osteoclast cells) may be modulated by OSCAR polypeptides of the present invention, including not only human OSCAR polypeptides, but also OSCAR polypeptides derived from other species of organism such as the mouse.

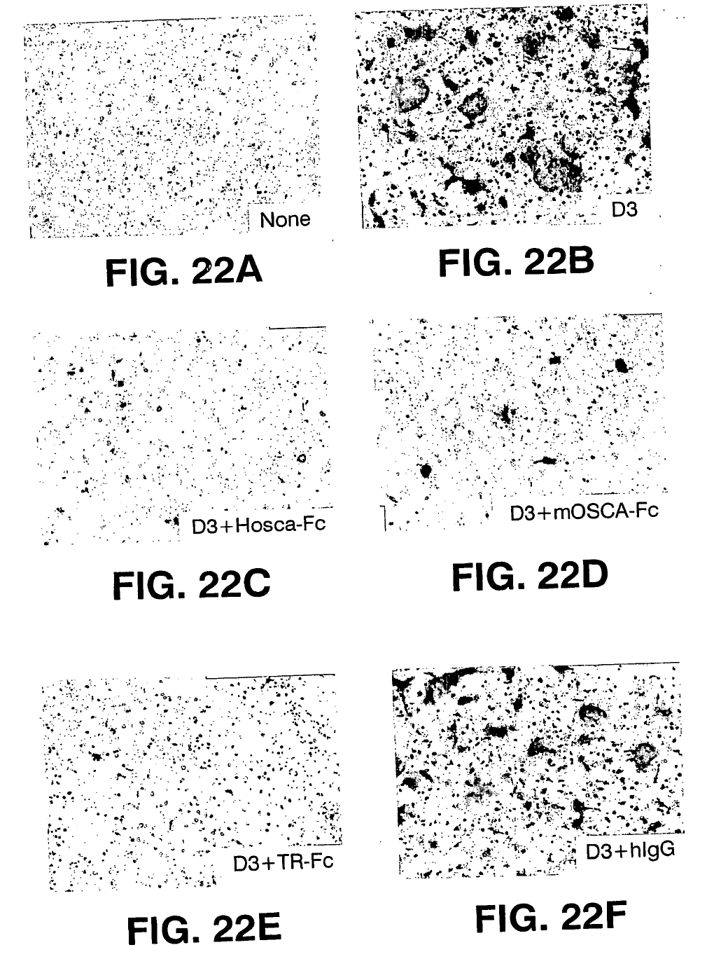

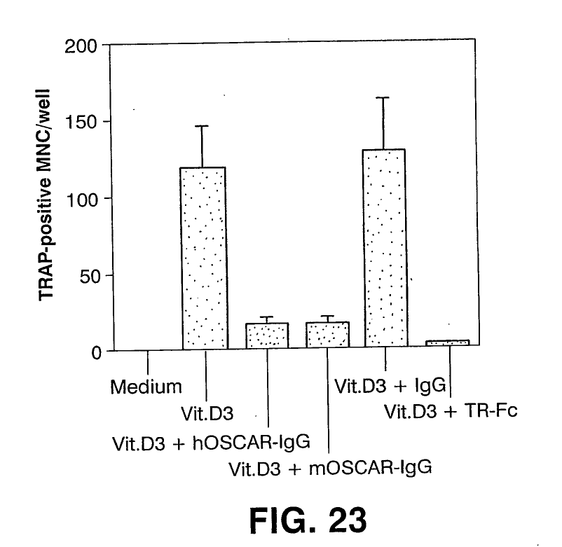

Human OSCAR is cross-reactive with murine cells. Converse experiments were also performed, that are similar to those described above using human monocyte cells, to investigate the ability of a human OSCAR polypeptide to modulate the maturation and activity of cells from other species of organisms. In particular, these experiments investigated the hOSCAR-Ig polypeptide's ability to modulate the maturation and activity of murine osteoclast cells. These experiments were essentially identical to the experiments described in Sections 4 and 5, supra using co-cultures of murine osteoblast and bone marrow cells. However, in these experiments the cell cultures were incubated with a soluble human OSCAR polypeptide (hOSCAR-Ig) rather than the soluble murine OSCAR polypeptide used in the previous examples. The results from these particular experiments are presented in FIGS. 22 and 23. Specifically, FIGS. 22A-22F show photomicrographs of the TRAP-stained murine cell cultures after incubating for six days with either growth medium alone (FIG. 22A), vitamin D₃ (FIG. 22B), vitamin D₃ and hOSCAR-Ig (FIG. 22C), or vitamin D₃ and mOSCAR-Ig (FIG. 22D). Positive and negative control experiments were also performed in which the co-cultures of murine cells were incubated either with vitamin D3 and an IgG1 polypeptide (FIG. 22F) or with vitamin D₃ and TR-Fc (FIG. 22E). The numbers of TRAP (+) multi-nuclear cells counted in each culture are shown graphically in FIG. 23. Consistent with what was observed in other experiments using murine cells, co-cultures that were incubated with vitamin D₃ and a murine OSCAR polypeptide had significantly fewer mature osteoclast cells, compared to numbers that were observed in co-cultures incubated with vitamin D₃ alone or with vitamin D₃ and a control IgG polypeptide. Interestingly, however, co-cultures that were incubated with vitamin D₃ and a human OSCAR polypeptide had similar levels of osteoclast cell inhibition.

The experiments described in this Example therefore demonstrate that the OSCAR nucleic acids and polypeptides of the present invention are cross-reactive, and may be used to modulate osteoclast cell maturation and/or activity in species of organisms that may be either the same as or different from the species of organism from which the OSCAR nucleic acid or polypeptide has been derived. Thus, OSCAR polypeptides and nucleic acids of the invention may be used to modulate process associated with the growth, development, repair, degradation, resorption or homeostasis of bone tissue in either the same species of organism as the species from which they have been derived, or in species of organisms that are different from the species from which they have been derived.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Numerous references, including patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entirety and to the same extent as if each reference was individually incorporated by reference.

What is claimed is:

1. An isolated OSCAR polypeptide.

2. The isolated polypeptide of claim 1 wherein the polypeptide is a murine polypeptide.

3. The isolated polypeptide of claim 2 comprising the amino acid sequence set forth in SEQ ID NO:5 (FIG. 2B).

4. The isolated polypeptide of claim 3 wherein the polypeptide comprises the amino acid sequence set forth in SEQ ID NO:3 (FIG. 1C).

5. The isolated polypeptide of claim 1 wherein the polypeptide is a human polypeptide.

6. The isolated polypeptide of claim 5 wherein the polypeptide is encoded by an OSCAR gene contained in the genomic sequence set forth in SEQ ID NO:12 (FIGS. 7A-D).

7. The isolated polypeptide of claim 6 wherein the polypeptide comprises the amino acid sequence set forth in SEQ ID NO:7 (FIG. 3B).

8. The isolated polypeptide of claim 6 wherein the polypeptide comprises the amino acid sequence set forth in SEQ ID NO:9 (FIG. 4B).

9. The isolated polypeptide of claim 6 wherein the polypeptide comprises the amino acid sequence set forth in SEQ ID NO:11 (FIG. 5B).

10. The isolated polypeptide of claim 1 comprising:
(a) the sequence of amino acid residues 1-16 of the amino acid sequence set forth in SEQ ID NO:3 (FIG. 1C);
(b) the sequence of amino acid residues 17-122 of the amino acid sequence set forth in SEQ ID NO:3 (FIG. 1C);
(c) the sequence of amino acid residues 123-228 of the amino acid sequence set forth in SEQ ID NO:3 (FIG. 1C);
(d) the sequence of amino acid residues 229-247 of the amino acid sequence set forth in SEQ ID NO:3 (FIG. 1C); or
(e) the sequence of amino acid residues 248-264 of the amino acid sequence set forth in SEQ ID NO:3 (FIG. 1C).

11. The isolated polypeptide of claim 1 comprising:
(a) the sequence of amino acid residues 1-18 of the amino acid sequence set forth in SEQ ID NO:17 (FIG. 3B);
(b) the sequence of amino acid residues 19-123 of the amino acid sequence set forth in SEQ ID NO:7 (FIG. 3B);
(c) the sequence of amino acid residues 124-229 of the amino acid sequence set forth in SEQ ID NO:7 (FIG. 3B);
(d) the sequence of amino acid residues 230-248 of the amino acid sequence set forth in SEQ ID NO:7 (FIG. 3B); or
(e) the sequence of amino acid residues 249-263 of the amino acid sequence set forth in SEQ ID NO:7 (FIG. 3B).

12. The isolated polypeptide of claim 1 comprising
(a) the sequence of amino acid residues 1-18 of the amino acid sequence set forth in SEQ ID NO:9 (FIG. 4B);
(b) the sequence of amino acid residues 19-127 of the amino acid sequence set forth in SEQ ID NO:9 (FIG. 4B);
(c) the sequence of amino acid residues 128-233 of the amino acid sequence set forth in SEQ ID NO:9 (FIG. 4B);
(d) the sequence of amino acid residues 234-252 of the amino acid sequence set forth in SEQ ID NO:9 (FIG. 4B); or
(e) the sequence of amino acid residues 253-267 of the amino acid sequence set forth in SEQ ID NO:9 (FIG. 4B).

13. The isolated polypeptide of claim 1 comprising:
(a) the sequence of amino acid residues 1-13 of the amino acid sequence set forth in SEQ ID NO:11 (FIG. 5B);
(b) the sequence of amino acid residues 14-112 of the amino acid sequence set forth in SEQ ID NO:11 (FIG. 5B);
(c) the sequence of amino acid residues 113-218 of the amino acid sequence set forth in SEQ ID NO:11 (FIG. 5B);
(d) the sequence of amino acid residues 219-237 of the amino acid sequence set forth in SEQ ID NO:11 (FIG. 5B); or
(e) the sequence of amino acid residues 238-252 of the amino acid sequence set forth in SEQ ID NO:11 (FIG. 5B).

14. An isolated polypeptide comprising an amino acid sequence encoded by a nucleic acid that hybridizes, under stringent conditions, to the complement of a nucleic acid encoding the polypeptide of claim 3.

15. The isolated polypeptide of claim 14 wherein the amino acid sequence is encoded by a nucleic acid that hybridizes to the complement of the nucleotide sequence set forth in SEQ ID NO:4 (FIG. 2A).

16. An isolated polypeptide comprising an amino acid sequence encoded by a nucleic acid that hybridizes, under stringent conditions, to the complement of a nucleic acid encoding the polypeptide of claim 4.

17. The iolsated polypeptide of claim 16 wherein the amino acid sequence is encoded by a nucleic acid that hybridizes to the complement of the nucleotide sequence set forth:
(a) in SEQ ID NO:1 (FIG. 1A); or
(b) in SEQ ID NO:2 (FIG. 1B).

18. An isolated polypeptide comprising an amino acid sequence encoded by a nucleic acid that hybridizes, under stringent conditions, to the complement of a nucleic acid encoding the polypeptide of claim 7.

19. The isolated polypeptide of claim 18 wherein the amino acid sequence is encoded by a nucleic acid that hybridizes to the complement of the nucleotide sequence set forth in SEQ ID NO:6 (FIG. 3A).

20. An isolated polypeptide comprising an amino acid sequence encoded by a nucleic acid that hybridizes, under stringent conditions, to the complement of a nucleic acid encoding the polypeptide of claim 8.

21. The isolated polypeptide of claim 20 wherein the amino acid sequence is encoded by a nucleic acid that hybridizes to the complement of the nucleotide sequence set forth in SEQ ID NO:8 (FIG. 4A).

22. An isolated polypeptide comprising an amino acid sequence encoded by a nucleic acid that hybridizes, under stringent conditions, to the complement of a nucleic acid encoding the polypeptide of claim 9.

23. The isolated polypeptide of claim 22 wherein the amino acid sequence is encoded by a nucleic acid that hybridizes to the complement of the nucleotide sequence set forth in SEQ ID NO:10 (FIG. 5A).

24. An isolated nucleic acid encoding an OSCAR polypeptide.

25. The isolated nucleic acid of claim 24 wherein the OSCAR polypeptide is a murine polypeptide.

26. The isolated nucleic acid of claim 25 which encodes a polypeptide comprising the amino acid sequence set forth in SEQ ID NO:5 (FIG. 2B).

27. The isolated nucleic acid of claim 20 wherein the nucleic acid comprises the nucleotide sequence set forth in SEQ ID NO:4 (FIG. 2A).

28. The isolated nucleic acid of claim 25 wherein the nucleic acid encodes a polypeptide comprising the amino acid sequence set forth in SEQ ID NO:3 (FIG. 1C).

29. The isolated nucleic acid of claim 28 wherein the nucleic acid comprises:
(a) the nucleotide sequence set forth in SEQ ID NO:1 (FIG. 1A); or
(b) the nucleotide sequence set forth in SEQ ID NO:2 (FIG. 1B).

30. The isolated nucleic acid of claim 24 wherein the OSCAR polypeptide is a human polypeptide.

31. The isolated nucleic acid of claim 30 which encodes a polypeptide comprising:
(a) the amino acid sequence set forth in SEQ ID NO:7 (FIG. 3B);
(b) the amino acid sequence set forth in SEQ ID NO:9 (FIG. 4B); or
(c) the amino acid sequence set forth in SEQ ID NO:11 (FIG. 5B).

32. The isolated nucleic acid of claim 31 wherein the nucleic acid comprises the nucleotide sequence set forth in SEQ ID NO:6 (FIG. 3A).

33. The isolated nucleic acid of claim 31 wherein the nucleic acid comprises the nucleotide sequence set forth in SEQ ID NO:8 (FIG. 4A).

34. The isolated nucleic acid of claim 31 wherein the nucleic acid comprises the nucleotide sequence set forth in SEQ ID NO:10 (FIG. 5A).

35. The isolated nucleic acid of claim 24 which encodes a polypeptide comprising:
(a) the sequence of amino acid residues 1-16 of the amino acid sequence set forth in SEQ ID NO:3 (FIG. 1C);
(b) the sequence of amino acid residues 17-122 of the amino acid sequence set forth in SEQ ID NO:3 (FIG. 1C);
(c) the sequence of amino acid residues 123-228 of the amino acid sequence set forth in SEQ ID NO:3 (FIG. 1C);
(d) the sequence of amino acid residues 229-247 of the amino acid sequence set forth in SEQ ID NO:3 (FIG. 1C); or
(e) the sequence of amino acid residues 248-264 of the amino acid sequence set forth in SEQ ID NO:3 (FIG. 1C).

36. The isolated nucleic acid of claim 24 which encodes a polypeptide comprising:
(a) the sequence of amino acid residues 1-18 of the amino acid sequence set forth in SEQ ID NO:17 (FIG. 3B);
(b) the sequence of amino acid residues 19-123 of the amino acid sequence set forth in SEQ ID NO:7 (FIG. 3B);
(c) the sequence of amino acid residues 124-229 of the amino acid sequence set forth in SEQ ID NO:7 (FIG. 3B);
(d) the sequence of amino acid residues 230-248 of the amino acid sequence set forth in SEQ ID NO:7 (FIG. 3B); or
(e) the sequence of amino acid residues 249-263 of the amino acid sequence set forth in SEQ ID NO:7 (FIG. 3B).

37. The isolated nucleic acid of claim 24 which encodes a polypeptide comprising:
(a) the sequence of amino acid residues 1-18 of the amino acid sequence set forth in SEQ ID NO:9 (FIG. 4B);
(b) the sequence of amino acid residues 19-127 of the amino acid sequence set forth in SEQ ID NO:9 (FIG. 4B);
(c) the sequence of amino acid residues 128-233 of the amino acid sequence set forth in SEQ ID NO:9 (FIG. 4B);
(d) the sequence of amino acid residues 234-252 of the amino acid sequence set forth in SEQ ID NO:9 (FIG. 4B); or
(e) the sequence of amino acid residues 253-267 of the amino acid sequence set forth in SEQ ID NO:9 (FIG. 4B).

38. The isolated nucleic acid of claim 24 which encodes a polypeptide comprising:
(a) the sequence of amino acid residues 1-13 of the amino acid sequence set forth in SEQ ID NO:11 (FIG. 5B);
(b) the sequence of amino acid residues 14-112 of the amino acid sequence set forth in SEQ ID NO:11 (FIG. 5B);
(c) the sequence of amino acid residues 113-218 of the amino acid sequence set forth in SEQ ID NO:11 (FIG. 5B);
(d) the sequence of amino acid residues 219-237 of the amino acid sequence set forth in SEQ ID NO:11 (FIG. 5B); or
(e) the sequence of amino acid residues 238-252 of the amino acid sequence set forth in SEQ ID NO:11 (FIG. 5B).

39. The isolated nucleic acid of claim 30 consisting of:
(a) a genomic OSCAR nucleotide sequence as set forth in SEQ ID NO:12 (FIGS. 7A-D); and, optionally
(b) a non-endogenous nucleotide sequence that is not naturally associated with the genomic OSCAR nucleotide sequence.

40. The isolated nucleic acid of claim 39 wherein the genomic OSCAR nucleotide sequence consist of at least one nucleotide sequence selected from the group consisting of:
(a) the sequence of nucleotides 768-841 of the nucleotide sequence set forth in SEQ ID NO:12 (FIGS. 7A-D);
(b) the sequence of nucleotides 842-1818 of the nucleotide sequence set forth in SEQ ID NO: 12 (FIGS. 7A-D);
(c) the sequence of nucleotides 1819-1851 of the nucleotide sequence set forth in SEQ ID NO:12 (FIGS. 7A-D);
(d) the sequence of nucleotides 1852-1997 of the nucleotide sequence set forth in SEQ ID NO:12 (FIGS. 7A-D);
(e) the sequence of nucleotides 1998-2009 of the nucleotide sequence set forth in SEQ ID NO:12 (FIGS. 7A-D);
(f) the sequence of nucleotides 2010-4439 of the nucleotide sequence set forth in SEQ ID NO:12 (FIGS. 7A-D);
(g) the sequence of nucleotides 4440-4742 of the nucleotide sequence set forth in SEQ ID NO:12 (FIGS. 7A-D);
(h) the sequence of nucleotides 4743-5013 of the nucleotide sequence set forth in SEQ ID NO:12 (FIGS. 7A-D);
(i) the sequence of nucleotides 5014-5295 of the nucleotide sequence set forth in SEQ ID NO:12 (FIGS. 7A-D);
(j) the sequence of nucleotides 5296-5809 of the nucleotide sequence set forth in SEQ ID NO:12 (FIGS. 7A-D);
(k) the sequence of nucleotides 5810-6499 of the nucleotide sequence set forth in SEQ ID NO:12 (FIGS. 7A-D).

41. An isolated nucleic acid that hybridizes, under stringent conditions, to the complement of a nucleic acid encoding the polypeptide of claim 3.

42. The isolated nucleic acid of claim 41 which hybridizes to the complement of the nucleotide sequence set forth in SEQ ID NO:4 (FIG. 2A).

43. An isolated nucleic acid that hybridizes, under stringent conditions, to the complement of a nucleic acid encoding the polypeptide of claim 4.

44. The isolated nucleic acid of claim 43 which hybridizes to the complement of the nucleotide sequence set forth:
(a) in SEQ ID NO:1 (FIG. 1A); or (b) in SEQ ID NO:2 (FIG. 1B).

45. An isolated nucleic acid that hybridizes, under stringent conditions, to the complement of a nucleic acid encoding the polypeptide of claim 7.

46. The isolated nucleic acid of claim 45 which hybridizes to the complement of the nucleotide sequence set forth in SEQ ID NO:6 (FIG. 3A).

47. An isolated nucleic acid that hybridizes, under stringent conditions, to the complement of a nucleic acid encoding the polypeptide of claim 8.

48. The isolated nucleic acid of claim 47 which hybridizes to the complement of the nucleotide sequence set forth in SEQ ID NO:8 (FIG. 4A).

49. An isolated nucleic acid that hybridizes, under stringent conditions, to the complement of a nucleic acid encoding the polypeptide of claim 9.

50. The isolated nucleic acid of claim 49 which hybridizes to the complement of the nucleotide sequence set forth in SEQ ID NO:10 (FIG. 5A).

51. An isolated nucleic acid which hybridizes, under stringent conditions, to the complement of the nucleic acid of claim 39.

52. An expression vector comprising the nucleic acid of claim 24 operatively associated with an expression control sequence.

53. An expression vector comprising the nucleic acid of claim 28 operatively associated with an expression control sequence.

54. An expression vector comprising the nucleic acid of claim 30 operatively associated with an expression control sequence.

55. An expression vector comprising the nucleic acid of claim 35 operatively associated with an expression control sequence.

56. An expression vector comprising the nucleic acid of claim 36 operatively associated with an expression control sequence.

57. An expression vector comprising the nucleic acid of claim 37 operatively associated with an expression control sequence.

58. An expression vector comprising the nucleic acid of claim 38 operatively associated with an expression control sequence.

59. A host cell genetically modified to express the nucleic acid of claim 24.

60. A host cell genetically modified to express the nucleic acid of claim 28.

61. A host cell genetically modified to express the nucleic acid of claim 30.

62. A host cell genetically modified to express the nucleic acid of claim 35.

63. A host cell genetically modified to express the nucleic acid of claim 36.

64. A host cell genetically modified to express the nucleic acid of claim 37.

65. A host cell genetically modified to express the nucleic acid of claim 38.

66. An isolated antibody that specifically binds to an OSCAR polypeptide.

67. An isolated antibody that specifically binds to the polypeptide of claim 3.

68. The antibody of claim 67 which is a monoclonal antibody.

69. An isolated antibody that specifically binds to the polypeptide of claim 4.

70. The antibody of claim 69 which is a monoclonal antibody.

71. An isolated antibody that specifically binds to the polypeptide of claim 7.

72. The antibody of claim 71 which is a monoclonal antibody.

73. An isolated antibody that specifically binds to the polypeptide of claim 8.

74. The antibody of claim 73 which is a monoclonal antibody.

75. An isolated antibody that specifically binds to the polypeptide of claim 9.

76. The antibody of claim 75 which is a monoclonal antibody.

77. A method for increasing activity of an osteoclast cell, which method comprises contacting the osteoclast cell with a compound that increases activity of an OSCAR gene product expressed by the osteoclast cell.

78. The method of claim 77 wherein the OSCAR gene product comprises a polypeptide having:
(a) the amino acid sequence set forth in SEQ ID NO:3 (FIG. 1C);
(b) the amino acid sequence set forth in SEQ ID NO:5 (FIG. 2B);
(c) the amino acid sequence set forth in SEQ ID NO:7 (FIG. 3B);
(d) the amino acid sequence set forth in SEQ ID NO:9 (FIG. 4B);
(e) the amino acid sequence set forth in SEQ ID NO:11 (FIG. 5B).

79. The method of claim 77 wherein the compound is an OSCAR-specific ligand.

80. The method of claim 79 wherein the compound is an antibody that specifically binds to the OSCAR gene product.

81. A method for increasing bone resorption, which method comprises increasing activity of an osteoclast cell according to the method of claim 77.

82. A method for decreasing activity of an osteoclast cell, which method comprises contacting the osteoclast cell with a compound that decreases activity of an OSCAR gene product expressed by the osteoclast cell.

83. The method of claim 82 wherein the OSCAR gene product comprises a polypeptide having:
(a) the amino acid sequence set forth in SEQ ID NO:3 (FIG. 1C);
(b) the amino acid sequence set forth in SEQ ID NO:5 (FIG. 2B);
(c) the amino acid sequence set forth in SEQ ID NO:7 (FIG. 3B);
(d) the amino acid sequence set forth in SEQ ID NO:9 (FIG. 4B); or
(e) the amino acid sequence set forth in SEQ ID NO:11 (FIG. 5B).

84. The method of claim 82 wherein the compound interferes with binding of an OSCAR specific ligand to the OSCAR gene product.

85. The method of claim 84 wherein the compound comprises a soluble OSCAR polypeptide.

86. The method of claim 85 wherein the soluble OSCAR polypeptide comprises:
(a) the sequence of amino acid residues 17-122 of the amino acid sequence set forth in SEQ ID NO:3 (FIG. 1C);
(b) the sequence of amino acid residues 123-228 of the amino acid sequence set forth in SEQ ID NO:3 (FIG. 1C);
(c) the sequence of amino acid residues 19-123 of the amino acid sequence set forth in SEQ ID NO:7 (FIG. 3B);
(d) the sequence of amino acid residues 124-229 of the amino acid sequence set forth in SEQ ID NO:7 (FIG. 3B);
(e) the sequence of amino acid residues 19-127 of the amino acid sequence set forth in SEQ ID NO:9 (FIG. 4B);
(f) the sequence of amino acid residues 128-233 of the amino acid sequence set forth in SEQ ID NO:9 (FIG. 4B);
(g) the sequence of amino acid residues 14-112 of the amino acid sequence set forth in SEQ ID NO:11 (FIG. 5B); or
(h) the sequence of amino acid residues 113-218 of the amino acid sequence set forth in SEQ ID NO:11 (FIG. 5B).

87. The method of claim 84 in which the soluble OSCAR polypeptide is a fusion polypeptide.

88. The method of claim 84 wherein the compound comprises:
(a) an antibody that specifically binds to the OSCAR gene product; or
(b) an antibody that specifically binds to the OSCAR specific ligand.

89. A method for decreasing bone resorption, which method comprises decreasing activity of an osteoclast cell according to the method of claim 82.

90. A method for identifying a cell as an osteoclast cell, which method comprises detecting expression of an OSCAR gene by the cell, wherein detection of expression of the OSCAR gene identifies the cell as an osteoclast cell.

91. The method of claim 90 wherein expression of the OSCAR gene is detected by detecting an mRNA encoding an OSCAR polypeptide.

92. The method of claim 90 wherein expression of the OSCAR gene is detected by detecting an OSCAR polypeptide.

93. A method for identifying a compound that binds to an OSCAR polypeptide, which method comprises:
(a) contacting a test compound to an OSCAR polypeptide under conditions sufficient to allow the test compound to bind to the OSCAR polypeptide; and
(b) detecting the test compound bound to the OSCAR polypeptide, wherein detection of the test compound bound to the OSCAR polypeptide identifies the test compound as a compound that binds to an OSCAR polypeptide.

94. The method of claim 93 wherein the OSCAR polypeptide comprises:
(a) the amino acid sequence set forth in SEQ ID NO:3 (FIG. 1C);
(b) the amino acid sequence set forth in SEQ ID NO:5 (FIG. 2B);
(c) the amino acid sequence set forth in SEQ ID NO:7 (FIG. 3B);
(d) the amino acid sequence set forth in SEQ ID NO:9 (FIG. 4B);
(e) the amino acid sequence set forth in SEQ ID NO:11 (FIG. 5B);
(f) the sequence of amino acid residues 17-122 of the amino acid sequence set forth in SEQ ID NO:3 (FIG. 1C);
(g) the sequence of amino acid residues 123-228 of the amino acid sequence set forth in SEQ ID NO:3 (FIG. 1C);
(h) the sequence of amino acid residues 19-123 of the amino acid sequence set forth in SEQ ID NO:7 (FIG. 3B);
(i) the sequence of amino acid residues 124-229 of the amino acid sequence set forth in SEQ ID NO:7 (FIG. 3B);
(j) the sequence of amino acid residues 19-127 of the amino acid sequence set forth in SEQ ID NO:9 (FIG. 4B);
(k) the sequence of amino acid residues 128-233 of the amino acid sequence set forth in SEQ ID NO:9 (FIG. 4B);
(j) the sequence of amino acid residues 14-112 of the amino acid sequence set forth in SEQ ID NO:11 (FIG. 5B); or
(m) the sequence of amino acid residues 113-218 of the amino acid sequence set forth in SEQ ID NO:11 (FIG. 5B).

95. The method of claim 93 wherein the test compound is a polypeptide.

96. The method of claim 95 wherein the polypeptide is expressed by an osteoblast cell, an embryonic fibroblast cell, an NIH 3T3 fibroblast cell, an ST2 osteoblast-like cell, a lung epithelial cell, a UMR106 cell, an HEK293 cell, an HEK293T cell, an hFOB1.19 cell, or a COS-1 cell.

97. A method for treating a bone growth related disorder in an individual, which method comprises increasing bone resorption in the individual according to the method of claim 81.

98. The method of claim 97 wherein the bone growth related disorder is osteopetrosis.

99. A method for treating a bone growth related disorder in an individual, which method comprises decreasing bone resorption in the individual according to the method of claim 89.

100. The method of claim 99 wherein the bone growth related disorder is osteoporosis.

101. The isolated polypeptide of claim 2 comprising the amino acid sequence set forth in SEQ ID NO:29 (FIG. 26B).

102. The isolated polypeptide of claim 2 comprising the amino acid sequence set forth in SEQ ID NO:31 (FIG. 27B).

103. The isolated polypeptide of claim 5 comprising the amino acid sequence set forth in SEQ ID NO:25 (FIG. 24B).

104. The isolated polypeptide of claim 5 comprising the amino acid sequence set forth in SEQ ID NO:27 (FIG. 25B).

105. The isolated nucleic acid of claim 25 wherein the nucleic acid comprises the nucleotide sequence set forth in SEQ ID NO:30 (FIG. 26A).

106. The isolated nucleic acid of claim 25 wherein the nucleic acid comprises the nucleotide sequence set forth in SEQ ID NO:32 (FIG. 27A).

107. The isolated nucleic acid of claim 30 wherein the nucleic acid comprises the nucleotide sequence set forth in SEQ ID NO:26 (FIG. 24A).

108. The isolated nucleic acid of claim 30 wherein the nucleic acid comprises the nucleotide sequence set forth in SEQ ID NO:28 (FIG. 25A).
